(19) United States
(12) Patent Application Publication
(10) Pub. No.: US 2011/0264026 A1
(43) Pub. Date: Oct. 27, 2011

(54) Azo Derivatives and Uses Thereof in Phototherapy

(76) Inventor: Raghavan Rajagopalan, St. Peters, MO (US)

(21) Appl. No.: 12/679,972

(22) PCT Filed: Nov. 23, 2009
(86) PCT No.: PCT/US09/65482

§ 371 Date: Mar. 25, 2010

Related U.S. Application Data
(60) Provisional application No. 61/117,310, filed on Nov. 24, 2008.

(51) Int. Cl. A61M 37/00; A61K 31/5025; C07D 498/06

(52) U.S. Cl. 604/20; 544/234; 514/248

(57) ABSTRACT

The invention relates generally to optical agents, including Type 1 phototherapeutic agents, for biomedical applications, such as phototherapy. Provided are fused ring azo and diaza compounds comprising a plurality of fused rings including a first ring having an intra-ring azo or intra-ring diaza group capable of activation upon exposure to electromagnetic radiation in visible and/or infrared regions of the electromagnetic spectrum. Optical agents of the invention enable a versatile phototherapy platform for treatment of a range of pathological conditions, including the treatment of cancers, stenosis and inflammation. The invention further provides preparations and formulations comprising the fused ring azo and diaza compounds and methods of making and using the fused ring azo and diaza compounds as optical agents in in vivo or ex vivo biomedical procedures.

### CROSS-REFERENCE TO RELATED APPLICATIONS

This application claims the benefit of U.S. Provisional Patent Application No. 61/117,310 filed on Nov. 24, 2008, which is hereby incorporated by reference in its entirety to the extent not inconsistent with the present description.

### INTRODUCTION

Optical agents currently play a central role in a large number of in vivo, in vitro and ex vivo clinical procedures including important diagnostic and therapeutic procedures. Photodiagnostic and phototherapeutic agents, for example, include a class of molecules capable of absorbing, emitting, or scattering electromagnetic radiation applied to a biological material, particularly in the visible and near infrared regions of the electromagnetic spectrum. This property of optical agents is used in a range of biomedical applications for visualizing, imaging or otherwise characterizing biological materials and/or achieving a desired therapeutic outcome. Recent developments in targeted administration and delivery of optical agents, and advanced systems and methods for applying and detecting electromagnetic radiation in biological environments has considerably expanded the applicability and effectiveness of optical agents for clinical applications.

Important applications of optical agents that absorb and/or emit in the visible and near-infrared (NIR) region of the electromagnetic spectrum include their use in biomedical imaging and visualization. For example, compounds absorbing and/or emitting electromagnetic radiation in these regions of the electromagnetic spectrum currently are useful for optical tomography, optoacoustic tomography, optical coherence tomography, confocal scanning laser tomography, optical coherence tomography, and fluorescence endoscopy; techniques which have emerged as essential molecular imaging techniques for imaging and visualizing biological processes at the organ, cellular and subcellular (e.g., molecular) levels. Biomedical images are generated, for example, by detecting electromagnetic radiation, nuclear radiation, acoustic waves, electrical fields, and/or magnetic fields transmitted, emitted and/or scattered by components of a biological sample. Modulation of the energy or intensity of the applied radiation yields patterns of transmitted, scattered and/or emitted radiation, acoustic waves, electrical fields or magnetic fields that contain useful anatomical, physiological, and/or biochemical information. A number of applications of biomedical imaging have matured into robust, widely used clinical techniques including planar projection and tomographic X-ray imaging, magnetic resonance imaging, ultrasound imaging, and gamma ray imaging.

Established optical imaging and visualization techniques are based on monitoring spatial variations in a variety of optical parameters including the intensities, polarization states, and frequencies of transmitted, reflected, and emitted electromagnetic radiation. Given that many biological materials of interest are incompatible with ultraviolet electromagnetic radiation, research is currently directed to developing and enhancing imaging techniques using visible and near infrared (NIR) radiation (from about 400 nm to about 900 nm). In particular, NIR electromagnetic radiation (700 nm to 900 nm) is useful for visualizing and imaging deeper regions than visible electromagnetic radiation because electromagnetic radiation of this wavelength range is capable of substantial penetration (e.g., up to four centimeters) in a range of biological media. Optical imaging and visualization using optical agents has potential to provide a less invasive and safer imaging technology, as compared to X-ray, and other widely used nuclear medicine technologies. Applications of optical imaging for diagnosis and monitoring of the onset, progression and treatment of various disease conditions, including cancer, are well established. (See, e.g., D. A. Benaron and D. K. Stevenson, Optical time-of-felectromagnetic radiation and absorbance imaging of biologic media, Science, 1993, 259, pp. 1463-1466; R. F. Potter (Series Editor), Medical optical tomography: functional imaging and monitoring, SPIE Optical Engineering Press, Bellingham, 1993; G. J. Tearney et al., In vivo endoscopic optical biopsy with optical coherence tomography, Science, 1997, 276, pp. 2037-2039; B. J, Tromberg et al., Non-invasive measurements of breast tissue optical properties using frequency-domain photon migration, Phil. Trans. Royal Society London B, 1997, 352, pp. 661-668; S. Fantini et al., Assessment of the size, position, and optical properties of breast tumors in vivo by noninvasive optical methods, Appl. Opt., 1998, 37, pp. 1982-1989; A. Pelegrin at al., Photoimmunodiagnosis with antibody-fluorescein conjugates: in vitro and in vivo preclinical studies, J. Cell Pharmacol., 1992, 3, pp. 141-145).

Optical agents for in vivo and in vitro biomedical imaging, anatomical visualization and monitoring organ function are described in International Patent Publication WO2008/108941; U.S. Pat. Nos. 5,672,333; 5,698,397; 6,167,297; 6,228,344; 6,748,259; 6,838,074; 7,011,817; 7,128,896, and 7,201,892. In this context, optical imaging agents are commonly used for enhancing signal-to-noise and resolution of optical images and extending these techniques to a wider range of biological settings and media. In addition, use of optical imaging agents having specific molecular recognition and/or tissue targeting functionality has also been demonstrated as effective for identifying, differentiating and characterizing discrete components of a biological sample at the organ, tissue, cellular, and molecular levels. Further, optical agents have been developed as tracers for real time monitoring of physiological function in a patient, including fluorescence-based monitoring of renal function. (See International Patent Publication PCT/US2007/0149478). Given their recognized utility, considerable research continues to be directed toward developing improved optical agents for biomedical imaging and visualization.

In addition to their important role in biomedical imaging and visualization, optical agents capable of absorption in the visible and NIR regions have also been extensively developed for clinical applications for phototherapy. The benefits of phototherapy using optical agents are widely acknowledged as this technique has the potential to provide efficacy comparable to radiotherapy, while entirely avoiding exposure of non-target organs and tissue to harmful ionizing radiation. Photodynamic therapy (PDT), in particular, has been used effectively for localized superficial or endoluminal malignant and premalignant conditions. The clinical efficacy of PDT has also been demonstrated for the treatment of various other diseases, injuries, and disorders, including cardiovascular disorders such as atherosclerosis and vascular restenosis, inflammatory diseases, ophthalmic diseases and dermatological diseases. Visudyne and Photofrin, for example, are two optical agents that have been developed for the treatment of macular degeneration of the eye and for ablation of several types of tumors, respectively. (See, e.g., Schmidt-Drfurth, U.; Bringruber, R.; Hasan, T. Phototherapy in ocular vascular disease. IEEE Journal of Selected Topics in Quantum Electronics 1996, 2, 988-996; Mlkvy, P.; Messmann, H.; Regula, J.; Conio, M.; Pauer, M.; Millson, C. E.; MacRobert, A. J.; Brown, S. G. Phototherapy for gastrointestinal tumors using three photosensitizers—ALA induced PPIX, Photofrin, and MTHPC. A pilot study. Neoplasma 1998, 45, 157-161; Grosjean, P.; Wagieres, G.; Fontolliet, C.; Van Den Bergh, H.; Monnier, P. Clinical phototherapy for superficial cancer in the esophagus and the bronchi: 514 nm compared with 630 nm electromagnetic radiation irradiation after sensitization with Photofrin II. British Journal of Cancer 1998, 77, 1989-1955; Milton, D.; Ackroyd, R. Phototherapy of Barrett's oesophagus and oesophageal carcinoma—how I do it. Photodiagnostics and Phototherapy 2006, 3, 96-98; and Li, L.; Luo, R.; Liao, W.; Zhang, M.; Luo, Y.; Miao, J. Clinical study of photofrin phototherapy for the treatment of relapse nasopharyngeal carcinoma. Photodiagnostics and Phototherapy 2006, 3, 266-271; See, Zheng Huang “A Review of Progress in Clinical Photodynamic Therapy”, Technol Cancer Res Treat. 2005 June; 4(3): 283-293; “Photodiagnosis And Photodynamic Therapy”, Brown S, Brown E A, Walker I. The present and future role of photodynamic therapy in cancer treatment. Lancet Oncol. 2004; 5:497-508; Triesscheijn M, Baas P, Schellens J H M. “Photodynamic Therapy in Oncology”; The Oncologist. 2006; 11:1034-1044; and Dougherty T J, Gomer C J, Henderson B W, Jori G, Kessel D, Korbelik M, Moan J, Peng Q. Photodynamic Therapy. J. Natl. Cancer Inst. 1998; 90:899-905).

Phototherapy is carried out by administration and delivery of a photosensitizer to a therapeutic target tissue (e.g., tumor, lesion, organ, etc.) followed by photoactivation of the photosensitizer by exposure to applied electromagnetic radiation. Phototherapeutic procedures require photosensitizers that are relatively chemically inert, and become activated only upon irradiation with electromagnetic radiation of an appropriate wavelength. Selective tissue injury can be induced with electromagnetic radiation when photosensitizers bind to the target tissues, either directly or through attachment to a bioactive carrier or targeting moiety. Photosensitizers essentially operate via two different pathways, classified as Types 1 and 2. A primary distinction between these classes of photosensitizers is that the Type 1 process operates via direct energy or electron transfer from the photosensitizer to the cellular components thereby inducing cell death, whereas the Type 2 process involves first the conversion of singlet oxygen from the triplet oxygen found in the cellular environment followed by either direct reaction of singlet oxygen with the cellular components or further generating secondary reactive species (e.g. peroxides, hydroxyl radical, etc.) which will induce cell death.

The Type 1 mechanism proceeds via a multistep process involving activation of the photosensitizer by absorption of electromagnetic radiation followed by direct interaction of the activated photosensitizer, or reactive species derived from the photosensitizer, with the target tissue, for example via energy transfer, electron transfer or reaction with reactive species (e.g., radicals, ions, nitrene, carbene etc.) resulting in tissue damage. The Type 1 mechanism can be schematically represented by the following sequence of reactions:

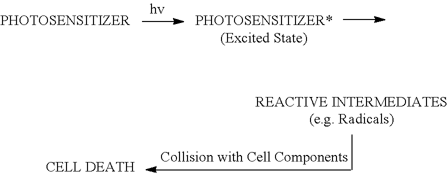

wherein hv indicates applied electromagnetic radiation and (PHOTOSENSITIZER)* indicates excited state of the photosensitizer. The Type 2 mechanism proceeds via a multi-step process involving activation of the photosensitizer by absorption of electromagnetic radiation followed by energy transfer from the activated photosensitizer to oxygen molecules in the environment of the target tissue. This energy transfer process generates excited state oxygen (¹O₂) which subsequently interacts with the target tissue so as to cause tissue damage. The Type 2 mechanism can be schematically represented by the following sequence of reactions:

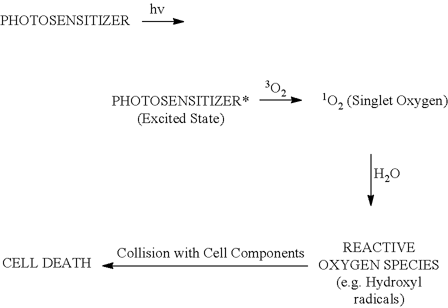

wherein hv indicates applied electromagnetic radiation, (PHOTOSENSITIZER)* indicates photoactivated photosensitizer, ³O₂ is ground state triplet oxygen, and ¹O₂ is excited state singlet oxygen.

The biological basis of tissue injury brought about by tumor phototherapeutic agents has been the subject of intensive study. Various biochemical mechanisms for tissue damage have been postulated, which include the following: a) cancer cells up-regulate the expression of low density lipoprotein (LDL) receptors, and phototherapy (PDT) agents bind to LDL and albumin selectively; (b) porphyrin-like substances are selectively taken up by proliferative neovasculature; (c) tumors often contain increased number of lipid bodies and are thus able to bind to hydrophobic photosensitizers; (d) a combination of “leaky” tumor vasculature and reduced lymphatic drainage causes porphyrin accumulation referred to as “EPR” (enhanced permeability and retention) effect; (e) tumor cells may have increased capabilities for phagocytosis or pinocytosis of porphyrin aggregates; (f) tumor associated macrophages may be largely responsible for the concentration of photosensitizers in tumors; and (g) cancer cells may undergo apoptosis induced by photosensitizers. Among these mechanisms, (f) and (g) are the most general and, of these two alternatives, there is a general consensus that (f) is the most likely mechanism by which the phototherapeutic effect of porphyrin-like compounds is induced.

Much of the research in the past several decades has focused on developing phototherapeutic agents based on the Type 2 (PDT) mechanism. Surprisingly, there has been considerably less attention devoted to Type 1 phototherapeutic agents despite the fact that there are numerous classes of compounds that could potentially be useful for phototherapy that function via this mechanism. Unlike Type 2, the Type 1 process does not require oxygen; and hence Type 1 photosensitizers are expected to be potentially more effective than Type 2 photosensitizers under hypoxic environments typically found in solid tumors. Second, the Type 1 mechanism involves two steps (photoexcitation and direct energy transfer), whereas the Type 2 mechanism involves three steps (photoexcitation, singlet oxygen generation, and energy transfer). Further, studies have recently shown that production of high levels of reactive oxygen species can induce an anti-inflammatory response, which may result in blood vessels to become more “leaky,” thereby increasing the risk of metastasis (Chen, B.; Pogue, B.; Luna, J. M.; Hardman, R. L.; Hoopes, P. J.; Hasan, T. Tumor vascular permeabilization by vascular-targeting photosensitization: effects, mechanism, and therapeutic implications. Clinical Cancer Research 2006, 12(3, Pt. 1), 917-923). Targeted Type 1 photosensitizers, by their very nature, are not expected to produce reactive oxygen species; rather, the reactive species produced by these photosensitizers will immediately react with the cellular component at the binding site and trigger cell death. Type 2 phototherapeutic agents, however, do have certain advantages over Type 1 agents. For example, Type 2 agents can potentially be catalytic, i.e., the Type 2 photosensitizer is regenerated once the energy transfer to the oxygen has taken place. In contrast, Type 1 process would generally be expected to require stoichiometric amounts of the photosensitizer in some clinical settings. Table I provides a summary of the attributes of Type 1 and Type 2 phototherapeutic agents. Given these attributes, it is clear that development of safe and effective Type 1 phototherapeutic agents would be useful to complement the existing therapeutic approaches provided by Type 2 agents, and to enhance the therapeutic portfolio available for clinicians.

TABLE 1
Comparison between Type 1 and Type
2 processes for phototherapy.

| TYPE 1 PROCESS | TYPE 2 PROCESS |
|---|---|
| Two-step process. | Three-step process. |
| Not well explored. | Very well studied. |
| Electromagnetic radiation of any | Requires red electromagnetic |
| wavelength can be used. | radiation for optimal performance. |
| Does not require oxygen. | Requires oxygen. |
| Large classes of compounds. | Limited classes of compounds. |
| Stoichiometric. | Potentially catalytic. |
| Intramolecular energy transfer to | Intermolecular energy transfer to |
| generate reactive species. | generate reactive oxygen species. |
| No products in the market. | Two products are in use. |

Specific optical, chemical and pharmacokinetic properties of optical agents are necessary for their effective use in Type 1 and Type 2 phototherapeutic applications. For example, optical agents for these applications preferably have strong absorption in the visible or NIR regions, and also exhibit low systemic toxicity, low mutagenicity, and rapid clearance from the blood stream. These optical agents must also be compatible with effective administration and delivery to the target tissue, for example by having reasonable solubilities and a low tendency for aggregation in solution. Upon excitation by absorption of visible and NIR electromagnetic radiation, optical agents for Type 1 and 2 phototherapy preferably provide large yields of singlet oxygen (Type 2) or other reactive species, such as free radicals or ions, capable of causing local tissue damage. Both Type 1 and Type 2 photosensitizers typically undergo photoactivation followed by intersystem crossing to their lowest triplet excited state, and therefore, a relatively long triplet lifetime is usually beneficial for providing effective tissue damage. Other useful properties of optical agents for these applications include chemical inertness and stability, insensitivity of optical properties to changes in pH, and compatibility with conjugation to ligands providing targeted delivery via molecular recognition functionality. Multifunctional optical agents have also been developed for phototherapy that are capable of providing both imaging and visual functionality upon excitation at a first range of wavelengths and phototherapeutic functionality upon excitation at a second range of wavelength. (See, U.S. Pat. No. 7,235,685 and International Patent Publication WO 2007/106436).

Optical agents for some phototherapeutic applications preferably exhibit a high degree of selectivity for the target tissue. Selectivity provided by optical agents facilitates effective delivery to a target tissue of interest and provides a means of differentiating different tissue classes during therapy. Selective tissue injury can be induced with electromagnetic radiation when photosensitizers bind to the target tissues either directly, as in the case of Photofrin, or through attachment to a bioactive carrier, or through in situ biochemical synthesis of the photosensitizer in localized area, as in the case of 2-aminolevulinic acid, which is an intermediate in the biosynthesis of porphyrin. Previous studies have shown that certain dyes selectively localize in tumors and serve as a powerful probe for the detection and treatment of small cancers. (D. A. Belinier et al., Murine pharmacokinetics and antitumor efficacy of the photodynamic sensitizer 2-[1-hexyloxyethyl]-2-devinyl pyropheophorbide-a, J. Photochem. Photobiol., 1993, 20, pp. 55-61; G. A. Wagnieres et al., In vivo fluorescence spectroscopy and imaging for oncological applications, Photochem, Photobiol., 1998, 68, pp. 603-632; J. S. Reynolds et al., Imaging of spontaneous canine mammary tumors using fluorescent contrast agents, Photochem. Photobiol., 1999, 70, pp. 87-94). It is recognized in some situations, however, that many dyes do not localize preferentially in malignant tissues. A number of strategies have been developed for imparting selectivity and/or targeting functionality by incorporation of a molecular recognition component in the optical agent. For example, targeting of fluorescent dyes to tumors has been demonstrated using dye conjugates with antibodies and peptides for diagnostic imaging of tumors. (See, Achilefu et al., Novel receptor-targeted fluorescent contrast agents for in vivo imaging of tumors, Investigative Radiology, 2000, 35, pp. 479-485; Ballou et al., Tumor labeling in vivo using cyanine conjugated monoclonal antibodies, Cancer Immunology and Immunotherapy, 1995, 41, pp. 257-263; and Licha et al., New contrast agent for optical imaging: acid cleavable conjugates of cyanine dyes with biomolecules, in Biomedical Imaging: Reporters, Dyes and Instrumentation, Proceedings of SPIE, 1999, 3600, pp. 29-35). Therefore, receptor-target mediated phototherapy agents provide a promising pathway for achieving site selective activation at various target tissues.

As will be generally recognized from the foregoing, a need currently exists for optical agents for biomedical applications. Specifically, optical agents for imaging, visualization and phototherapy are needed having enhanced specificity for important target tissue classes, such as tumors and other lesions. In addition, optical agents are needed having enhanced optical, physical, chemical and pharmacokinetic properties for administration, delivery and excitation with electromagnetic radiation.

### SUMMARY

The invention relates generally to optical agents, including Type 1 phototherapeutic agents, for biomedical applications, such as phototherapy. Provided are fused ring azo and diaza compounds comprising a plurality of fused rings including a first ring having an intra-ring azo or intra-ring diaza group capable of activation upon exposure to electromagnetic radiation having wavelengths in visible and/or infrared regions of the electromagnetic spectrum. Optical agents of the invention enable a versatile phototherapy platform for treatment of a range of pathological conditions, including the treatment of cancers, stenosis and inflammation. The invention further provides preparations and formulations comprising the fused ring azo and diaza compounds and methods of making and using the fused ring azo and diaza compounds as optical agents in in vivo or ex vivo biomedical procedures.

In some embodiments, for example, the invention provides fused ring azo and diaza compounds for phototherapeutic methods having one or more a photolabile intra-ring C—N, N—N and/or N═N bonds that undergo photoactivation upon exposure to electromagnetic radiation to produce reactive species, such as radicals, nitrenes, carbenes, ions, etc., for achieving a desired therapeutic effect, such as selective and/or localized tissue damage and/or cell death, inactivation or injury. For example, optical agents of an aspect of the invention include azo compounds comprising a first ring having a cyclic N═N bond, a second unsaturated ring and a third aromatic ring, all of which provided in a fused ring configuration; and diaza compounds comprising a first ring having a cyclic N—N bond, a second unsaturated ring and a third aromatic ring, all of which provided in a fused ring configuration. Optical agents further include conjugates, for example, fused ring azo and diaza compounds including a targeting ligand such as an aptamer, polypeptide, oligonucleotide, carbohydrate, antibody, or other biomolecule, or fragments thereof, capable of providing molecular recognition and/or targeting functionality. Optical agents further include multifunctional optical agents providing tandem phototherapy and imaging functionality comprising a fused ring azo or diaza component functioning as a photosensitizer directly or indirectly linked to a chromophore component, such as a C₅-C₃₀ aryl group, functioning as an optically detectable component.

In an aspect, the invention provides a class of fused ring azo and diaza compounds useful as optical agents for phototherapeutic methods, including Type 1 phototherapy, comprising a first ring having an intra-ring azo or intra-ring diaza group, wherein the first ring is fused to a second unsaturated ring and third aromatic ring. In an embodiment, for example, the invention provides a compound for use in a phototherapy procedure, the compound being of the formula (FX1):

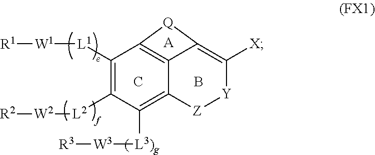

or a pharmaceutically acceptable salt or ester thereof, wherein:

Y is —CU^{a}U^{b}—, —NU^{a}—, —O—, —S—, or —C(O)—;

Z is —CU^{c}U^{d}—, —NU^{c}—, —O—, —S—, or —C(O)—;

wherein U^{a} is independently -(L⁴)ₕ-W⁴—R⁴;

wherein U^{b} is independently -(L⁵)₁-W⁵—R⁵;

wherein U^{c} is independently -(L⁶)ⱼ-W⁶—R⁶;

wherein U^{d} is independently -(L⁷)ₖ-W⁷—R⁷;

X is hydrogen, F, Cl, Br, I, or At;

Q is —C(R⁸R⁹)N═N—, —C(R⁸)═NN(R⁹)—, or —N═N—,

each of L¹-L⁷, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;

each of W¹-W⁷ is independently a single bond, —(CH₂)ₙ—, —(HCCH)ₙ—, —O—, —S—, —SO—, —SO₂—, —SO₃—, —OSO₂—, —NR¹⁴—, —CO—, —COO—, —OCO—, —OCOO—, —CONR¹⁵—, —NR¹⁶CO—, —OCONR¹⁷—, —NR¹⁸COO—, —NR¹⁹CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ, —OCO(CH₂)ₙ—, —OCOO(CH₂)ₙ—, —CONR²⁴(CH₂)ₙ—, —CONR²⁵(CH₂)ₙ—, —NR²⁶CO(CH₂)ₙ—, —OCONR²⁷(CH₂)ₙ—, —NR²⁸COO(CH₂)ₙ—, —NR²⁹CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—;

each of R¹-R⁹ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₂₀ alkoxy, C₁-C₂₀ alkoxycarbonyl, C₁-C₂₀ alkoxyalkyl, C₁-C₂₀ polyhydroxyalkyl, C₁-C₂₀ polyalkoxyalkyl, halo, halomethyl, dihalomethyl, trihalomethyl, —CO₂R⁴⁰, —SOR⁴¹, —OSR⁴², —SO₂OR⁴³, —CH₂(CH₂OCH₂)_{b}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —SO₃R⁶², —CH(R⁶³)CO₂H, —CH(R⁶⁴)NH₂, FL or Bm; or wherein R¹, R², W¹, W², and L¹ and L², if present, together with the atoms to which they are attached combine to form one or more carbocyclic or heterocyclic 5, 6, or 7 membered rings; or wherein R², R³, W², W³, and L² and L³, if present, together with the atoms to which they are attached combine to form one or more carbocyclic or heterocyclic 5, 6, or 7 membered rings; or wherein U^{a} and U^{c} together with the atoms to which they are attached combine to form one or more carbocyclic or heterocyclic 5, 6, or 7 membered rings; or wherein R³, W³, and L³, if present, and U^{c} together with the atoms to which they are attached combine to form one or more carbocyclic or heterocyclic 5, 6, or 7 membered rings;

each of a and b is independently an integer selected from the range of 1 to 100;

each of n is independently an integer selected from the range of 1 to 10;

each of e, f, g, h, i, j, and k is independently 0 or 1;

each of R¹⁴-R³⁶ is independently hydrogen, C₁-C₁₀ alkyl, C₁-C₁₀ alkoxy, C₅-C₁₀ aryl, C₁-C₁₀ alkoxyalkyl, C₁-C₁₀ polyhydroxyalkyl, —(CH₂)ₙCO₂R⁶⁵, or —COR⁶⁶;

each of R⁴⁰-R⁶² and R⁶⁵-R⁶⁶ is independently hydrogen, C₁-C₁₀ alkyl, C₁-C₁₀ alkoxy, C₁-C₁₀ alkoxyalkyl, or C₁-C₁₀ polyhydroxyalkyl;

each of R⁶³ and R⁶⁴ is independently a side chain residue of a natural α-amino acid;

each of FL is independently a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine; and

each Bm is independently a group corresponding to an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steriod, a hormone, an aptamer, a receptor, a metal chelating agent, a mono- or polynucleotide comprising 1 to 50 nucleic acid units, or a polypeptide comprising 2 to 30 amino acid units.

In an embodiment, the composition of R¹-R⁹, Q, X, Y, Z, W¹-W⁹ and L¹-L⁹ is selected such that the compound undergoes photoactivation upon exposure to electromagnetic radiation having wavelengths over the range of 350 nanometers to 1300 nanometers, and optionally wavelengths over the range of 400 nanometers to 900 nanometers. In an embodiment, for example, at least one, and optionally all, of R¹-R³ is an electron donating group or electron withdrawing group. In an embodiment, for example, at least one, and optionally all, of R⁴-R⁷ is a targeting ligand (Bm). In an embodiment, for example, X is a halogen, such as Br.

As used throughout the present description, reference to embodiments wherein e, f, g, h, i, j, and k is equal to 0 refers to compounds where L¹, L², L³, L⁴, L⁵, L⁶, and/or L⁷, respectively, is not present, and reference to embodiments wherein e, f, g, h, i, j, and k is equal to 1 refers to compounds where L¹, L², L³, L⁴, L⁵, L⁶, and/or L⁷, respectively, is present. For example, W¹ is directly linked to the central fused ring core of formula (FX1) when e is equal to 0; and/or W² is directly linked to the central fused ring core of formula (FX1) when f is equal to 0; and/or W³ is directly linked to the central fused ring core of formula (FX1) when g is equal to 0; and/or W⁴ is directly linked to the central fused ring core of formula (FX1) when h is equal to 0; and/or W⁵ is directly linked to the central fused ring core of formula (FX1) when i is equal to 0; and/or W⁶ is directly linked to the central fused ring core of formula (FX1) when j is equal to 0; and/or W⁷ is directly linked to the central fused ring core of formula (FX1) when k is equal to 0. Embodiments wherein W¹ is a single bond and e is equal to 0 refer to compositions having R¹ directly linked to the fused ring core of formula (FX1) via a single bond. Embodiments wherein W² is a single bond and f is equal to 0 refer to compositions having R² directly linked to the fused ring core of formula (FX1) via a single bond. Embodiments wherein W³ is a single bond and g is equal to 0 refer to compositions having R³ directly linked to the fused ring core of formula (FX1) via a single bond. Embodiments wherein W⁴ is a single bond and h is equal to 0 refer to compositions having R⁴ directly linked to the fused ring core of formula (FX1) via a single bond. Embodiments wherein W⁵ is a single bond and i is equal to 0 refer to compositions having R⁵ directly linked to the fused ring core of formula (FX1) via a single bond. Embodiments wherein W⁶ is a single bond and j is equal to 0 refer to compositions having R⁶ directly linked to the central fused ring core of formula (FX1) via a single bond. Embodiments wherein W⁷ is a single bond and k is equal to 0 refer to compositions having R⁷ directly linked to the central fused ring core of formula (FX1) via a single bond. As used herein, the expression “fused ring core” refers to fused rings A, B and C as provided in formula (FX1). As used throughout the present description, the expression “a group corresponding to” an indicated species expressly includes a radical (including a divalent radical), for example an aromatic radical or heterocyclic aromatic radical, of the species or group of species provided in a covalently bonded configuration, optionally with one or more ring substituents, including but not limited to electron donating groups, electron withdrawing groups, fluorophores, photosensitizers and/or targeting ligands.

The invention provides fused ring azo compounds useful as optical agents for phototherapeutic methods wherein Q is —N═N— and ring A is a 5 membered ring, the compound being of the formula (FX2):

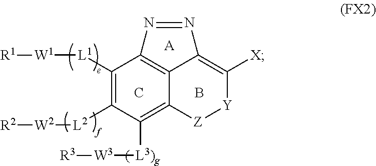

or a pharmaceutically acceptable salt or ester thereof, wherein: R¹-R³, L¹-L³, W¹-W³, e, f, g, X, Y and Z are as described in connection with formula (FX1). The invention provides fused ring diaza compounds useful as optical agents for phototherapeutic methods wherein Q is —C(R⁸)═NN(R⁹)— and ring A is a 6 membered ring, the compound being of the formula (FX3):

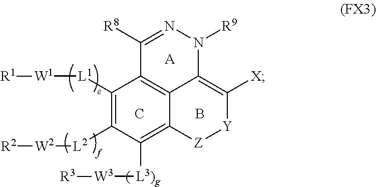

or a pharmaceutically acceptable salt or ester thereof, wherein: R¹-R³, R⁸, R⁹, L¹-L³, W¹-W³, e, f, g, X, Y and Z are as described in connection with formula (FX1). The invention provides fused ring azo compounds useful as optical agents for phototherapeutic methods wherein Q is —C(R⁸R⁹)N═N— and ring A is a 6 membered ring, the compound being of the formula (FX4):

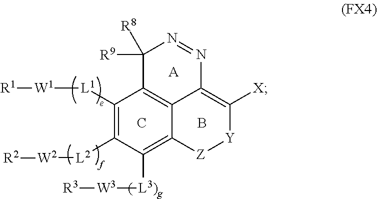

or a pharmaceutically acceptable salt or ester thereof, wherein: R¹-R³, R⁸, R⁹, L¹-L³, W¹-W³, e, f, g, X, Y and Z are as described in connection with formula (FX1).

Compounds of the invention include isomers, for example diaza and azo isomers having formulas (FX3) and (FX4), respectively. Compounds of the invention include tautomers having formula (FX3) and (FX4) that are capable of undergoing tuatomerization reactions that interconvert between tautomeric forms, for example via hydrogen migration and/or photoactivated tautomerization. In an embodiment wherein both of R⁸ or R⁹ are not hydrogen, for example, the ground state of a compound of the invention has formula (FX4). In another embodiment wherein either of R⁸ or R⁹ is hydrogen, for example, the compound of the invention preferentially exists in its tautomeric diaza form indicated by structure (FX3) in the ground state. Scheme 1 below provides an example of a compound of the invention having formula (FX4) wherein R⁹ is hydrogen, wherein the azo compound undergoes interconversion to form the diaza tautomer having formula (FX3):

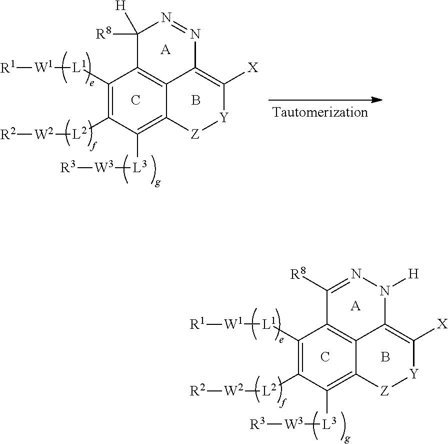

In another embodiment, for example, photoactivation of a diaza compound of the invention results in formation of an excited state corresponding to the azo tautomer. Scheme 2 below provides an example of a diaza compound of the invention having formula (FX3) wherein R⁹ is hydrogen, wherein photoactivation of the diaza compound forms the azo tautomer having formula (FX4):

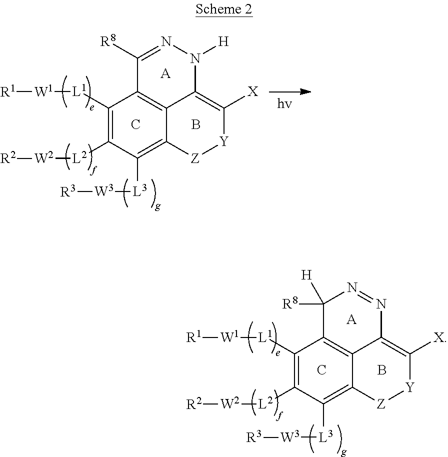

The invention further provides fused ring azo and diaza compounds useful as optical agents for phototherapeutic methods wherein ring B is a 6 membered carbocyclic ring, the compound being of the formula (FX5), (FX6), (FX7), or (FX8):

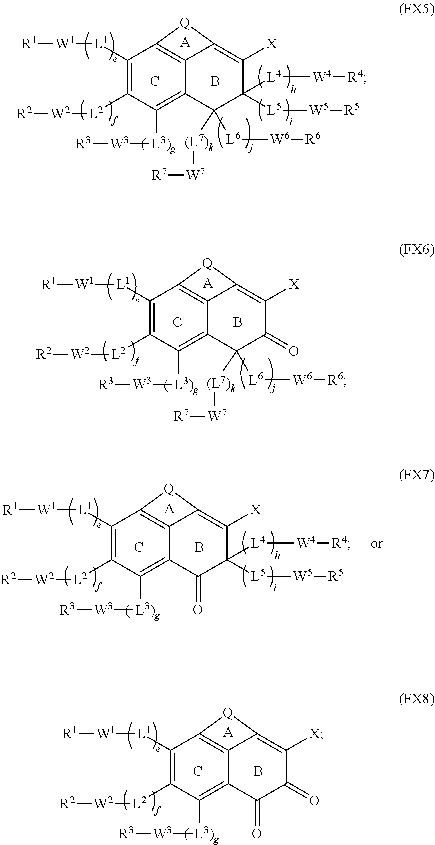

or a pharmaceutically acceptable salt or ester thereof, wherein: R¹-R⁷, L¹-L⁷, W¹-W⁷, Q, X, e, f, g, h, i, j, and k are as described in connection with formula (FX1).

The invention further provides fused ring azo and diaza compounds useful as optical agents for phototherapeutic methods, wherein ring B is a 6 membered heterocyclic ring, the compound being of the formula (FX9),(FX10), (FX11), (FX12), (FX13) (FX14), (FX15), (FX16), (FX17), (FX18), (FX19) or (FX20):

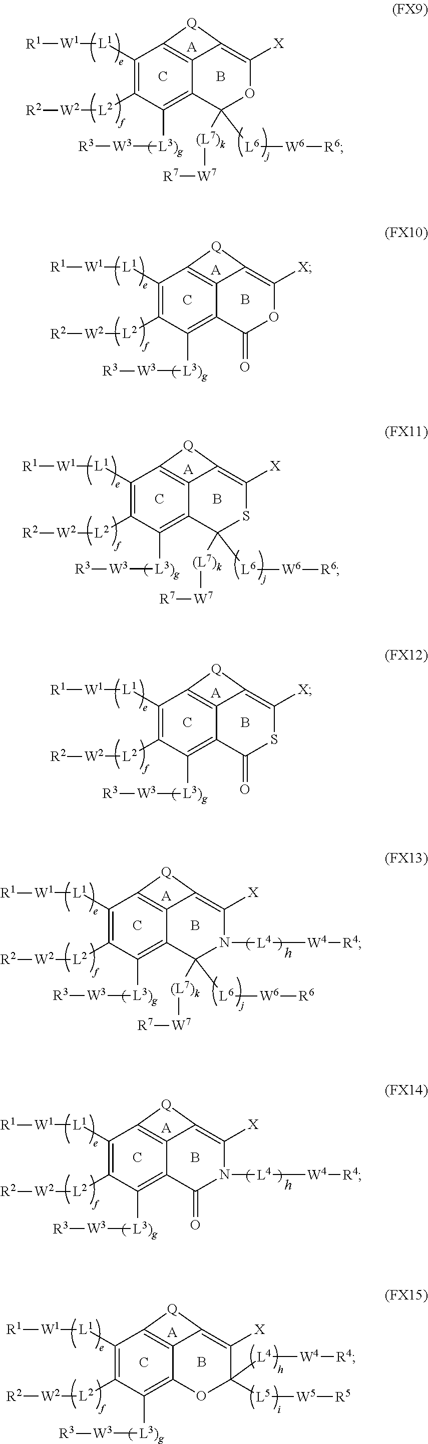

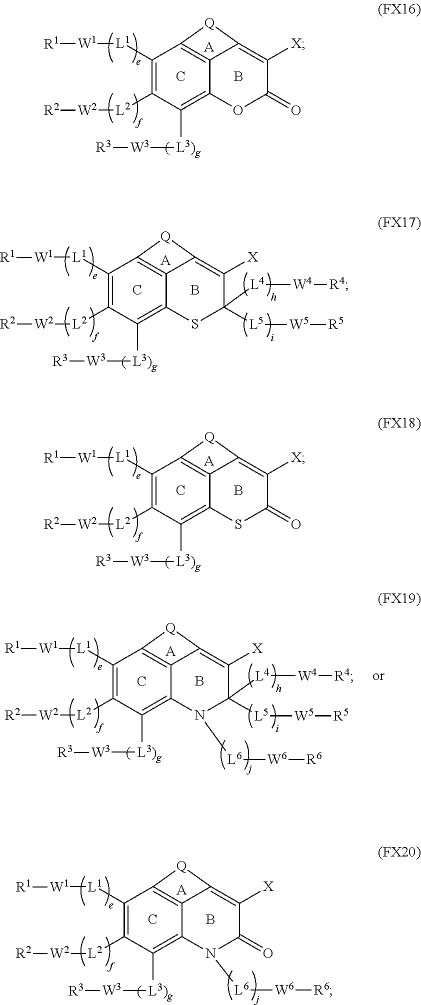

or a pharmaceutically acceptable salt or ester thereof, wherein R¹-R⁷, L¹-L⁷, W¹-W⁷, Q, X, e, f, g, h, i, j, and k are as described in connection with formula (FX1).

The invention further provides compounds useful as optical agents for phototherapeutic methods, wherein one or more ring substituents of rings C and B combine to form one or more additional rings fused to rings C and/or B. In an embodiment, for example, the invention provides fused ring azo and diaza compounds for phototherapy, wherein R¹, R², W¹, W², and L¹ and L², if present, together with the atoms to which they are attached combine to form one or more carbocyclic or heterocyclic 5, 6, or 7 membered rings, optionally one carbocyclic or heterocyclic 5, 6, or 7 membered rings; and/or wherein R², R³, W², W³, and L² and L³, if present, together with the atoms to which they are attached combine to form one or more carbocyclic or heterocyclic 5, 6, or 7 membered rings optionally one carbocyclic or heterocyclic 5, 6, or 7 membered rings. In an embodiment, for example, the invention provides fused ring diaza and azo compounds useful as optical agents for phototherapy being of the formula (FX21), (FX22), or (FX23):

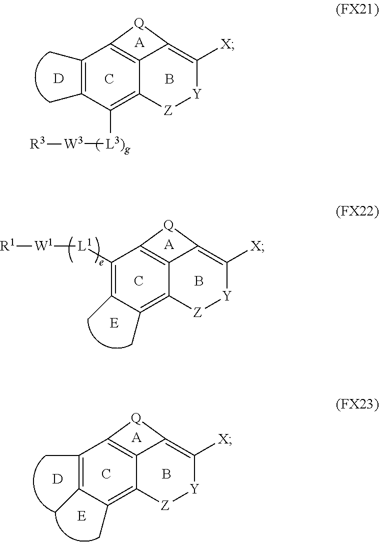

or a pharmaceutically acceptable salt or ester thereof, wherein each of rings D and E is independently one or more carbocyclic or heterocyclic 5, 6, or 7 membered rings; and wherein R¹-R³, L¹-L³, W¹-W³, Q, X, Y, Z, e, and g, are as described in connection with formula (FX1). In an embodiment, each of rings D and E of formula (FX21)-(FX23) is independently a carbocyclic or heterocyclic aromatic ring, such as a pyrazine group of a phenyl group. In an embodiment, for example, the invention provides a fused ring azo or diaza compound having any one of formula (FX21)-(FX23), wherein each of rings D and E is independently a C₅-C₂₀ aryl group, optionally a C₅-C₁₀ aryl group, fused to ring C. In an embodiment, for example, each of rings D and E of formula (FX21)-(FX23) is independently a group corresponding to benzene, naphthalene, naphthoquinone, diphenylmethane, fluorene, anthracene, anthraquinone, phenanthrene, tetracene, naphthacenedione, pyridine, quinoline, isoquinoline, indole, isoindole, pyrrole, imidazole, oxazole, thiazole, pyrazole, pyrazine, pyrimidine, purine, benzimidazole, furan, benzofuran, dibenzofuran, carbazole, acridine, acridone, phenanthridine, thiophene, benzothiophene, dibenzothiophene, xanthene, xanthone, flavone, coumarin, azulene, aza-azulene, or anthracycline, optionally having one or more electron withdrawing groups, electron donating groups and/or targeting ligands provided as ring substituents. In an embodiment, for example, the invention provides a fused ring azo or diaza compound having any one of formula (FX21)-(FX23), wherein each of rings D and E is independently a group corresponding to benzene, pyrazine, azulene or aza-azulene, optionally having one or more electron withdrawing groups, electron donating groups and/or targeting ligands provided as ring substituents.

In an embodiment, for example, the invention provides fused ring azo and diaza compounds for phototherapy, wherein U^{a} and U^{c} together with the atoms to which they are attached combine to form one or more carbocyclic or heterocyclic 5, 6, or 7 membered rings, optionally one carbocyclic or heterocyclic 5, 6, or 7 membered rings; and/or wherein R³, W³, and L³, if present, and U^{c} together with the atoms to which they are attached combine to form one or more carbocyclic or heterocyclic 5, 6, or 7 membered rings, optionally one carbocyclic or heterocyclic 5, 6, or 7 membered rings. In an embodiment, for example, the invention provides fused ring diaza and azo compounds useful as optical agents for phototherapy being of the formula (FX24), (FX25), (FX26), (FX27) or (FX28):

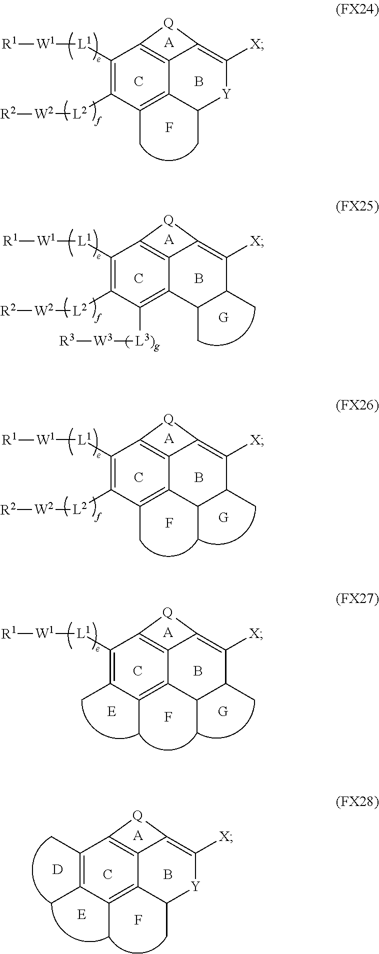

or a pharmaceutically acceptable salt or ester thereof, wherein each of rings F and G is independently one or more carbocyclic or heterocyclic 5, 6, or 7 membered rings; wherein R¹-R³, L¹-L³, W¹-W³, Q, X, Y, Z, e, f, g, h, i, j, and k, are as described in connection with formula (FX1), and wherein each of rings D and E of formula (FX24)-(FX28) are as defined in the description of formulas (FX21)-FX23). In an embodiment, each of rings F and G of formula (FX24)-(FX28) is independently one or more carbocyclic or heterocyclic alicyclic rings, and optionally a single carbocyclic or heterocyclic alicyclic ring. In an embodiment, for example, each of rings D and E of formula (FX24)-(FX28) is independently a carbocyclic or heterocyclic C₅-C₁₀ cycloalkyl or C₅-C₁₀ cycloalkenyl. In an embodiment, for example, each of rings D and E of formula (FX24)-(FX28) is independently cyclopentane, cyclohexane, cycloheptane or piperidine.

In an embodiment, for example, the invention provides fused ring azo and diaza compounds for phototherapy, wherein R¹, R², and R³ are directly attached to ring C; the compound being of the formula (FX29):

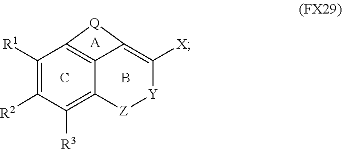

or a pharmaceutically acceptable salt or ester thereof, wherein Q, X, Y, Z, R¹, R², and R³ are as described in connection with formula (FX1).

In an embodiment, for example, the invention provides fused ring azo and diaza compounds for phototherapy of formula (FX30) or (FX31):

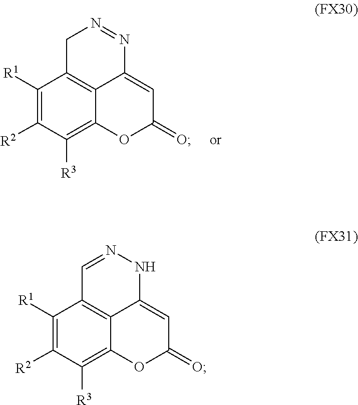

or a pharmaceutically acceptable salt or ester thereof, wherein R¹, R² and R³ are as defined in connection with formula (FX1). In an embodiment, the invention provides compounds having any of formulas (FX29)-(FX31), wherein each of R¹, R² and R³ is hydrogen, C₁-C₁₀ alkyl, C₅-C₁₀ aryl, —NR⁴⁸R⁴⁹, —CO₂R⁴⁰, —NR⁵⁰COR⁵¹, or Bm.). In an embodiment, the invention provides compounds having any of formulas (FX29)-(FX31), wherein each of R¹, R² and R³ is hydrogen or C₁-C₁₀ alkyl, and optionally wherein each of R¹, R² and R³ is hydrogen.

The invention includes therapeutic agents for biomedical applications, including phototherapy, comprising purified stereoisomers (e.g., enantiomers and diastereomers), tautomers (diaza and azo tautomers), salts (including quarternary salts), and/or ionic forms (e.g., protonated and deprotonated forms) of the compounds of any of formula (FX1)-(FX40), and mixtures thereof. As will be understood by those having general skill in the art, acidic functional groups and basic functional groups of the compounds of any of formula (FX1)-(FX40) may be in protonated or deprotonated states depending on the molecular environment (e.g., pH, ionic strength, composition, etc.), for example during synthesis, formulation and/or administration.

Fused ring azo and diaza compounds of the invention include unsaturated ring B fused to ring A which has an intra-ring azo or intra-ring diaza group and is fused to aromatic ring C. In an embodiment, unsaturated ring B has an intra-ring alkene group, wherein a carbon atom of the alkene group is also a ring member of ring A. In some embodiments, the presence of the intra-ring alkene group may enhance the stability of the fused ring azo or diaza compound prior to photoactivation, for example, under formulation, delivery and in vivo conditions. In some embodiments, unsaturated ring B is provided in a configuration so as to extend the overall conjugation in the compound, for example extending the conjugation of aromatic ring C. Extending conjugation via incorporation of unsaturated ring B has the benefit in some compounds of enabling the photoactivation and internal energy transfer processes to occur upon absorption of electromagnetic radiation having longer wavelengths, as compared to the unconjugated analog (e.g., an analog having saturated ring substituted for ring B), that results in generation of reactive species. Some compounds of the present invention, for example, have a red shifted absorption spectrum relative to corresponding compounds wherein ring B is substituted with a fully saturated 6 membered ring. Incorporation of unsaturated ring B in compounds of the invention is important for enabling phototherapy biomedical procedures using visible and NIR electromagnetic radiation, as opposed to ultraviolet electromagnetic radiation that can cause unwanted tissue damage upon application of electromagnetic radiation to a subject. Incorporation of unsaturated ring B in compounds of the invention is also significant as it allows use of visible and NIR electromagnetic radiation in a phototherapy procedure that is transmitted appreciably into biological media. In some embodiments, the invention provides fused ring azo and diaza compounds having any of formula (FX1)-(FX29) wherein X is hydrogen. In some embodiments, the invention provides fused ring azo and diaza compounds having any of formula (FX1)-(FX29), wherein X is a halogen atom, such as F, Cl, Br, or At. Compounds of the invention having formula (FX1)-(FX29), wherein X is a halogen atom, may be useful for generating reactive species comprising halogen radicals upon photoactivation.

In certain embodiments of the invention, the composition of ring substituents on the rings A, B, C, D, E, F and/or G in compositions having formula (FX1)-(FX40) is selected to achieve preselected properties, such as optical, physiochemical and pharmacokinetic properties useful for biomedical applications including phototherapy and optical imaging. As used herein, a ring substituent refers to an atom or moiety directly or indirectly bonded to an intra-ring atom (e.g., a ring member). The invention provides compounds of any one of formula (FX1)-(FX40), for example, wherein any of rings A, B, C, D, E, F and G has at least one electron donating group provided as a ring substituent and/or at least one electron withdrawing group provided as a ring substituent. In some embodiments, for example, any of rings B, C, D, E, F and G has at least one electron donating group and at least one electron withdrawing group provided as ring substituents. The invention provides, for example, compositions having any one of (FX1)-(FX40) wherein at least one of R¹, R² and/or R³ is an electron withdrawing group (EWG) bonded directly or indirectly to a carbon atom of ring C and at least one of R¹, R² and/or R³ is an electron donating group (EDG) bonded directly or indirectly to a carbon atom of ring C. Incorporation of a combination of an EWD and an EDG as ring substituents of different carbon atoms of any of rings B, C, D, E, F and G is particularly beneficial for providing optical agents having large extinction coefficients in the visible and near infrared regions of the electromagnetic spectrum (e.g., 350 nm-1300 nm, optionally 400 nm to 900 nm), emission in the visible and near infrared regions (e.g., 350 nm-1300 nm, optionally 500-900 nm), a large fluorescence quantum yield (e.g., >0.1) and a Stoke's shift useful for optical detection and imaging (e.g., Stoke's shift >10 nm). In some embodiments, for example, an electron withdrawing group and an electron donating group are positioned on adjacent carbon atoms of any of rings B, C, D, E, F and G. Alternatively, the invention includes embodiments wherein an electron withdrawing group and an electron donating group are positioned on non-adjacent carbon atoms of any of rings B, C, D, E, F and G. Multiple electron withdrawing groups and/or electron donating groups on each substituent arm of any of rings B, C, D, E, F and G are contemplated by the compositions of this aspect of the invention. By way of example, one EWG arm may comprise two, three, or more electron withdrawing groups bonded to any of rings B, C, D, E, F and G via a common linking moiety.

In an embodiment, the invention provides compositions having any one of formula (FX1)-(FX29), wherein at least one of R¹, R² and R³ is C₁-C₁₀ alkyl, —OR⁴⁶, —SR⁴⁷, and —NR⁴⁸R⁴⁹, and —NR⁵⁰COR⁵¹, and wherein optionally each of R⁴⁶-R⁵¹ is H or C₁-C₁₀ alkyl. In an embodiment, the invention provides compositions having any one of formula (FX1)-(FX29), wherein at least one of R¹, R² and R³ is —NR⁴⁸R⁴⁹, or —NR⁵⁰COR⁵¹, and wherein optionally each of R⁴⁸-R⁵¹ is H or C₁-C₁₀ alkyl. In an embodiment, the invention provides compositions having any one of formula (FX1)-(FX29), wherein at least one of R¹, R² and R³ is —CN, halo, —CO₂R⁴⁰, —COR⁵⁴, —NO₂, —SO₂R⁵⁵, or —SO₂NR⁵⁸R⁵⁹, and wherein optionally each of R⁴⁰, R⁵⁴, R⁵⁵, R⁵⁸ and R⁵⁹ is H or C₁-C₁₀ alkyl. In an embodiment, the invention provides compositions having any one of formula (FX1)-(FX29), wherein at least one of R¹, R² and R³ is —CO₂R⁴⁰, —COR⁵⁴, —SO₂NR⁵⁸R⁵⁹ or —SO₂R⁵⁵, and wherein optionally each of R⁴⁰, R⁵⁴, R⁵⁸ and R⁵⁹ is H or C₁-C₁₀ alkyl. In an embodiment, the invention provides compositions having any one of formula (FX1)-(FX29), wherein at least one of R¹, R² and R³ is —NR⁴⁸R⁴⁹, and wherein at least one of R¹, R² or R³ is —CO₂R⁴⁰, —COR⁵⁴, —SO₂NR⁵⁸R⁵⁹ or —SO₂R⁵⁵, and wherein optionally each of R⁴⁸, R⁴⁹, R⁴⁰, R⁵⁴, R⁵⁵, R⁵⁸ and R⁵⁹ is H or C₁-C₁₀ alkyl.

In an embodiment, the invention provides compounds having electron-donating and electron-withdrawing groups attached to adjacent positions of ring C. Alternatively, the invention includes compounds having electron-donating and electron-withdrawing groups attached to non-adjacent positions of ring C. In an embodiment, provided are compounds of formula (FX1)-(FX29) wherein:

(a) any one of R¹ and R² is C₁-C₆ alkyl, —OR⁴⁶, —SR⁴⁷, —NR⁴⁸R⁴⁹, or —NR⁵⁰COR⁵¹; and the other of R¹ and R² is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹;

(b) any one of R¹ and R³ is C₁-C₆ alkyl, —OR⁴⁶, —SR⁴⁷, —NR⁴⁸R⁴⁹, or —NR⁵⁰COR⁵¹; and the other of R¹ and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹;

(c) any one of R² and R³ is C₁-C₆ alkyl, —OR⁴⁶, —SR⁴⁷, —NR⁴⁸R⁴⁹, or —NR⁵⁰COR⁵¹; and the other of R² and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹:

(d) any two of R¹, R² and R³ is C₁-C₆ alkyl, —OR⁴⁶, —SR⁴⁷, —NR⁴⁸R⁴⁹, or —NR⁵⁰COR⁵¹; and the other of R¹, R² and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; or

(e) any two of R¹, R² and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; and the other of R¹, R² and R³ is C₁-C₆ alkyl, —OR⁴⁶, —SR⁴⁷, —NR⁴⁸R⁴⁹, or —NR⁵⁰COR⁵¹.

In an embodiment, the invention provides optical agents for phototherapy having a ligand component for targeting the optical agent to a selected organ, tissue, or other cell material. Incorporation of a targeting ligand in some compounds and methods of the invention enables targeted delivery such that at least a portion of optical agent administered to a subject preferentially accumulates at a preselected, desired site, such as the site of an organ, tissue, or tumor or other lesion, prior to, or during, exposure to electromagnetic radiation. In some embodiments, the targeting ligand of an optical agent selectively binds to, or otherwise preferentially associates with, biomolecules (e.g., proteins, carbohydrates, hormones, lipids, oligonucleotides, etc.) excreted or otherwise generated by a target tissue. Targeting ligands of the invention may be indirectly or directly linked to, or non-covalently associated with, the central fused ring core of formulas (FX1)-(FX29). In some embodiments, for example, the invention provides fused ring azo and diaza compounds having any one of formula (FX1)-(FX29) wherein at least one of rings A, B, C, D, E, F, and G has a targeting ligand provided as a substituent. The invention includes, for example, compounds of any one of formula (FX1)-(FX29), wherein at least one of R¹-R⁹ is independently a targeting ligand (abbreviated as “Bm” throughout this description). In an embodiment, for example, the invention includes compounds wherein R¹ is Bm and W¹ is —NR¹⁶CO—, —CONR¹⁵—OCONR¹⁷—, —NR¹⁸COO—, or —NR¹⁹CONR²⁰—; or R² is Bm and W² is —NR¹⁶CO—, —CONR¹⁵—OCONR¹⁷—, —NR¹⁸COO—, or —NR¹⁹CONR²⁰—; or R³ is Bm and W³ is —NR¹⁸CO—, —CONR¹⁵—OCONR¹⁷—, —NR¹⁸COO—, or —NR¹⁹CONR²⁰—; or R⁴ is Bm an W⁴ is —NR¹⁶CO—, —CONR¹⁵—OCONR¹⁷—, —NR¹⁸COO—, or —NR¹⁹CONR²⁰—; or R⁵ is Bm and W⁵ is —NR¹⁶CO—, —CONR¹⁵—OCONR¹⁷—, —NR¹⁸COO—, or —NR¹⁹CONR²⁰—; or R⁶ is Bm and W⁶ is —NR¹⁶CO—, —CONR¹⁵—OCONR¹⁷—, —NR¹⁸COO—, or —NR¹⁹CONR²⁰—; or R⁷ is Bm and W⁷ is —NR¹⁶CO—, —CONR¹⁵—OCONR¹⁷—, —NR¹⁸COO—, or —NR¹⁹CONR²⁰—. In an embodiment, for example, the invention includes compounds of any one of formula (FX1)-(FX29), wherein at least one of R¹-R⁹, optionally at least one of R⁴-R⁷, is independently a polypeptide comprising 2 to 30 amino acid units. In an embodiment, for example, the invention includes compounds of any one of formula (FX1)-(FX29), wherein at least one of R¹-R⁹, optionally at least one of R⁴-R⁷, is independently an antibody or fragment thereof. In an embodiment, for example, the invention includes compounds of any one of formula (FX1)-(FX29), wherein at least one of R¹-R⁹, optionally at least one of R⁴-R⁷, is independently a polynucleotide comprising 1 to 50 nucleic acid units. In an embodiment, for example, the invention includes compounds of any one of formula (FX1)-(FX29), wherein at least one of R¹-R⁹, optionally at least one of R⁴-R⁷, is independently an aptamer.

The invention includes compounds of any one of formula (FX1)-(FX29), wherein at least one of R¹-R⁹ is independently a dye (abbreviated as “FL”), such as a C₅-C₃₀ aryl chromophore and/or C₅-C₃₀ aryl fluorophore, that is excited upon exposure to electromagnetic radiation having wavelengths selected over the range of 350 nanometers to 1300 nanometers, optionally over the range of 400 nanometers to 900 nanometers. Compounds of this aspect of the invention include bifunctional optical agents, capable of providing tandem functionality as a photosensitizer and imaging agent. In an embodiment, for example, the invention provides a compound having any one of formula (FX1)-(FX29) that functions as a photosensitizer upon exposure to electromagnetic radiation having a first distribution of wavelengths, and wherein at least one of R¹-R⁹ is independently a fluorophore that is excited upon exposure to electromagnetic radiation having a second distribution of wavelengths that is different from the first distribution of wavelengths, for example, wherein the first and second distributions of wavelengths have different absorption maxima and, optionally wherein the first and second distributions of wavelengths are characterized by absorption peaks that are not overlapping or by absorption maxima in the visible or near IR regions of the electromagnetic spectrum that differ by 20 nanometers or more. In an embodiment, for example, at least one of R¹-R⁹ is independently a C₅-C₃₀ aryl fluorophore having one or more electron donating groups as substituents, having one or more electron withdrawing groups as substituents, or having both electron donating and electron withdrawing groups as substituents. In an embodiment, at least one of R¹-R⁹ is independently a fluorophore group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine, optionally having one or more electron donating groups, electron withdrawing groups, or targeting ligands provided as one or more substituents.

In an embodiment, the invention provides compounds of any one of formulas (FX1)-(FX29), wherein each of R¹⁴-R³⁶ is independently hydrogen, a C₁-C₁₀ alkyl, or C₁-C₁₀ alkoxy, and optionally wherein each of R¹⁴-R³⁶ is hydrogen, a C₁-C₅ alkyl or C₁-C₅ alkoxy, and optionally wherein each of R¹⁴-R³⁶ is hydrogen. In an embodiment, the invention provides compounds of any one of formulas (FX1)-(FX29), wherein each of R⁴⁰-R⁶² and R⁶⁵-R⁶⁶ is independently hydrogen, C₁-C₁₀ alkyl or C₁-C₁₀ alkoxy, and optionally wherein each of R⁴⁰-R⁶² and R⁶⁵-R⁶⁶ is hydrogen, C₁-C₅ alkyl and C₁-C₅ alkoxy, and optionally wherein each of R⁴⁰-R⁶² and R⁶⁵-R⁶⁶ is hydrogen. In an embodiment, the invention provides compounds useful as optical agents for phototherapeutic methods having any one of formulas (FX1)-(FX29), wherein each of R¹-R⁹ is independently hydrogen, C₁-C₁₀ alkyl, C₁-C₁₀ alkoxy, C₅-C₁₀ aryl, —CH(R⁶³)CO₂H, —OR⁴⁶, —CH(R⁶⁴)NH₂, FL or Bm. In an embodiment, the invention provides compounds useful as optical agents for phototherapeutic methods having any one of formulas (FX1)-(FX29), wherein R¹ is a C₅-C₁₀ aryl, wherein optionally the C₅-C₁₀ aryl includes an electron donating group, electron withdrawing group, and/or Bm as a ring substituent, and each of R² and R³ is independently a hydrogen, C₁-C₁₀ alkyl, C₅-C₁₀ aryl, a FL or Bm, wherein optionally the C₅-C₁₀ aryl includes an electron donating group, electron withdrawing group, or a targeting ligand (Bm) as a substituent. In an embodiment, the invention provides compounds useful as optical agents for phototherapeutic methods having any one of formulas (FX1)-(FX29), wherein R² is a C₅-C₁₀ aryl, wherein optionally the C₅-C₁₀ aryl includes an electron donating group, electron withdrawing group, and/or Bm as a ring substituent, and each of R¹ and R³ is independently a hydrogen, C₁-C₁₀ alkyl, C₅-C₁₀ aryl, a FL or Bm, wherein optionally the C₅-C₁₀ aryl includes an electron donating group, electron withdrawing group, or a targeting ligand (Bm) as a substituent. In an embodiment, the invention provides compounds useful as optical agents for phototherapeutic methods having any one of formulas (FX1)-(FX29), wherein R³ is a C₅-C₁₀ aryl, wherein optionally the C₅-C₁₀ aryl includes an electron donating group, electron withdrawing group, and/or Bm as a ring substituent, and each of R¹ and R² is independently a hydrogen, C₁-C₁₀ alkyl, C₅-C₁₀ aryl, a FL or Bm, wherein optionally the C₅-C₁₀ aryl includes an electron donating group, electron withdrawing group, or a targeting ligand (Bm) as a substituent. In an embodiment, the invention provides compounds useful as optical agents for phototherapeutic methods having any one of formulas (FX1)-(FX29), wherein at least one of R¹, R², and R³ is a FL, such as a fluorophore that is excited upon exposure to electromagnetic radiation having wavelengths over the range of 350 nanometers to 1300 nanometers, preferably wavelengths selected over the range of 400 nanometers to 900 nanometers for some applications.

L¹-L⁷ and W¹-W⁷ groups may be spacer and attaching groups, respectively, for providing an appropriate linkage between R¹-R⁷ and the fused ring core of formulas (FX1)-(FX29). In some embodiments, the invention provides compounds of any one of formulas (FX1)-(FX29), wherein any one of L¹-L⁷ is independently a spacer moiety for establishing the relationship between R¹-R⁷ and the fused ring core providing useful optical, pharmacokinetic, or targeting properties. In some embodiments, the invention provides compounds of any one of formulas (FX1)-(FX29), wherein any one of W¹-W⁷ is independently an attaching moiety for attaching R¹-R⁷ directly or indirectly to the fused ring core. In an embodiment, the invention provides compounds having formula (FX1)-(FX29), wherein at least one of e, f, g, h, i, j and k is 0, and optionally all of e, f, g, h, i, j and k are 0. In an embodiment, the invention provides compounds having formula (FX1)-(FX29), and related phototherapy methods, wherein e is 0, and/or f is 0, and/or g is 0, and/or h is 0, and/or i is 0, and/or j is 0, and/or k is 0. In an embodiment, the invention provides compounds useful as optical agents for phototherapeutic methods having any of formulas (FX1)-(FX29), wherein at least one of W¹-W⁷ is a single bond, and optionally all of W¹-W⁷ are single bonds. In an embodiment, the invention provides compounds having formula (FX1)-(FX29), wherein W¹-W⁷ is a single bond, and/or W² is a single bond, and/or W³ is a single bond, and/or W⁴ is a single bond, and/or W⁵ is a single bond, and/or W⁶ is a single bond, and/or W⁷ is a single bond. In an embodiment, the invention provides compounds having formula (FX1)-(FX29), and related phototherapy methods, wherein e is 0 and W¹ is a single bond and R¹ is directly bonded to ring C; and/or f is 0 and W² is a single bond and R² is directly bonded to ring C; and/or g is 0 and W³ is a single bond and R³ is directly bonded to ring C; and/or h is 0 and W⁴ is a single bond and R⁴ is directly bonded to ring B; and/or i is 0 and W⁵ is a single bond and R⁵ is directly bonded to ring B; and/or j is 0 and W⁶ is a single bond and R⁶ is directly bonded to ring B; and/or k is 0 and W⁷ is a single bond and R⁷ is directly bonded to ring B.

In an embodiment, at least one of L¹-L⁷ is independently —(CH₂)ₘ—, —(HCCH)ₘ—, —(CHOH)ₘ—, or —(CH₂CH₂O)ₘ—, wherein each m is independently an integer selected from the range of 1 to 10. In an embodiment, the invention provides compounds of any one of formulas (FX1)-(FX29), wherein at least one of W¹-W³ is independently a single bond, —O—, —CO—, —COO—, —OCO—, —OCOO—, —NR¹⁴—, —CONR¹⁵—, —NR¹⁶CO—; —NR¹⁹CONR²⁰—, or —NR²¹CSNR²²—. In an embodiment, the invention provides compounds of any one of formulas (FX1)-(FX29), wherein at least one of: L¹ and W¹; L² and W²; and L³ and W³; and L⁴ and W⁴; and L⁵ and W⁵; and L⁶ and W⁶; and L⁷ and W⁷ combine to form: —(CH₂)ₙ—, —O(CH₂)ₙ—, —CO(CH₂)ₙ—, —OCO(CH₂)ₙ—, —COO(CH₂)ₙ—, —OCOO(CH₂)ₙ—, —N(R²³)(CH₂)ₙ—, —CON(R²⁵)(CH₂)ₙ—, —N(R²⁶)CO(CH₂)ₙ—, —OCONR²⁷(CH₂)ₙ—, —NR²⁸COO(CH₂)ₙ—, —NR²⁹CONR³⁰(CH₂)ₙ—, or —NR³¹CSNR³²(CH₂)ₙ—, wherein each n is independently an integer selected from the range of 1 to 10.

In an embodiment, for example, the invention provides a compound for phototherapy having any one of formula (FX1)-(FX40), wherein R¹-R⁹ do not include an azide group. In an embodiment, for example, the invention provides a compound for phototherapy having any one of formula (FX1)-(FX40), wherein R¹-R⁹ do not include a sulfenate group. In an embodiment, for example, the invention provides a compound for phototherapy having any one of formula (FX1)-(FX40), wherein R¹-R⁹ do not include a thiadiazole group. In an embodiment, for example, the invention provides a compound for phototherapy having any one of formula (FX1)-(FX40), wherein R¹-R⁹ do not include a cyanate group. In an embodiment, for example, the invention provides a compound for phototherapy having any one of formula (FX1)-(FX40), wherein R¹-R⁹ do not include an isocyanide group. In an embodiment, for example, the invention provides a compound for phototherapy having any one of formula (FX1)-(FX40), wherein R¹-R⁹ do not include an isocyanate group. In an embodiment, for example, the invention provides a compound for phototherapy having any one of formula (FX1)-(FX40), wherein R¹-R⁹ do not include an isothiocyanate group. In an embodiment, for example, the invention provides a compound for phototherapy having any one of formula (FX1)-(FX40), wherein R¹-R⁹ do not include a thiocyanate group. In an embodiment, for example, the invention provides a compound for phototherapy comprising a ring having an intra-ring diaza or intra-ring azo group fused to additional rings that are not saturated rings.

In some embodiments, compounds of the invention may optionally include a poly(ethylene glycol) (abbreviated as PEG) component. In an embodiment, for example, the invention provides a composition having any one of the formula (FX1)-(FX29), wherein at least one of R¹-R⁹ is —(CH₂CH₂O)_{b}R⁵¹ and/or at least one of L¹-L⁷ is —(CH₂OCH₂)_{b}—, wherein b is selected from the range of 1 to 100. Incorporation of a poly(ethylene glycol) glycol component in some compositions of the invention provides pharmacokinetic, chemical, and/or physical properties useful for bioanalytical, diagnostic and/or phototherapeutic applications. Poly(ethylene glycol) containing compounds of some embodiments of the invention, for example, provide enhanced biocompatibility, low toxicity and suppress immune responses upon administration. Poly(ethylene glycol) containing compounds of some embodiments of the invention facilitate formulation, administration and/or delivery, for example, by enhancing solubility.

The invention further provides a compound having any one of formula (FX1)-(FX40), or a pharmaceutical formulation thereof, for use in an optical imaging, diagnostic, and/or phototherapeutic biomedical procedure such as a Type 1 or Type 2 phototherapy procedure. In an embodiment, the invention provides an optical agent comprising a pharmaceutically acceptable formulation, wherein at least one active ingredient of the formulation is a compound having any one of formula (FX1)-(FX40) provided in a therapeutically effective amount. The invention includes, for example, formulations comprising a compound having any one of formula (FX1)-(FX40) and one or more pharmaceutically acceptable carriers or excipients. In an embodiment, the invention provides a pharmaceutically acceptable formulation for combination therapy comprising a compound having any one of formula (FX1)-(FX40) and one or more additional diagnostic and/or therapeutic agents, such as anti-cancer agents, anti-inflammatory agents, and/or imaging agents (e.g., optical and/or non-optical imaging agents).

In an embodiment, the invention provides methods for a biomedical procedure, such as a phototherapy procedure, wherein the method comprises: (i) administering (e.g., via intravenous or intraarterial injection, oral administration, topical administration, subcutaneous administration, etc.) to a subject a therapeutically or diagnostically effective amount of the compound having any one of formula (FX1)-(FX40) and (ii) exposing the administered compound to electromagnetic radiation. In an embodiment, the administrating step is carried out under conditions sufficient for contacting the compound with a target tissue or cell, wherein the compound selectively binds to or otherwise preferentially associates with the target tissue or cell. In an embodiment, the administered compound is exposed to electromagnetic radiation having wavelengths selected over a range of 350 nanometers to 1300 nanometers, optionally having wavelengths selected over a range of 400 nanometers to 900 nanometers. In a specific embodiment, the administered compound is exposed to electromagnetic radiation having wavelengths selected over a range of 300 nanometers to 900 nanometers. In an embodiment, exposing the administered compound to electromagnetic radiation cleaves one or more C—N, N—N and/or N═N bonds in ring A of the compound, thereby generating reactive species. In an embodiment, exposing the administered compound to electromagnetic radiation generates a therapeutically effective amount of photoactivated compound or a diagnostically effective amount of photoactivated compound. In an embodiment, exposing the administered compound to electromagnetic radiation generates a therapeutically effective amount of reactive species causing localized cell death, inactivation or injury.

In an embodiment, the medical phototherapy procedure comprises administering, contacting or otherwise targeting the compound to or with a target tissue of the subject, such as a tumor, lesion, site of inflammation, vasculature tissue, or organ. In an embodiment, methods of the invention further comprise exposing the administered compound at the target tissue to electromagnetic radiation having sufficient power, fluence, intensity and/or dose (net number of photons provided to the target tissue) to result in injury, inactivation and/or death to cells at the target tissue. In an embodiment, for example, the target tissue is a tissue type selected from the group consisting of breast, lung, throat, cervical, colon, kidney, stomach, ovarian, testicular, prostate, gastric, esophageal, uterine, endometrial, and pancreatic tissue. In an embodiment, exposing the administered compound to electromagnetic radiation generates fluorescence, wherein the biomedical procedure further comprises detecting fluorescence from the administered compound. In an embodiment, exposing the administered compound to electromagnetic radiation generates a diagnostically effective amount of fluorescence, for example an amount of fluorescence allowing for optical detection, visualizing and/or imaging of the target tissue. In an embodiment, a method of the invention further comprises exposing the administered compound at the target tissue to electromagnetic radiation having sufficient power, fluence, intensity and/or dose (net number of photons provided to the target tissue) to provide optical detection, visualization and/or imaging of the target tissue. In an embodiment, a method of the invention further comprises generating an image of the fluorescence from the compound. In an embodiment, a method of the invention further comprises visualizing the fluorescence from the compound.

In a method, the electromagnetic radiation exposed to the compound of any one of formulas (FX1)-(FX40) does not have wavelengths in the X-ray region of the electromagnetic spectrum. In a method, the electromagnetic radiation exposed to the compound of any one of formulas (FX1)-(FX40) does not have wavelengths in the ultraviolet region of the electromagnetic spectrum. In an embodiment, non-ionizing electromagnetic radiation is used in the present methods. “Non-ionizing electromagnetic radiation” herein refers to electromagnetic radiation wherein a single photon does not have enough energy to completely remove at least one electron from an atom or molecule of the subject's body.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE FIGURES

Those of skill in the art will understand that the figures, described below, are for illustrative purposes only. The figures are not intended to limit the scope of the present teachings in any way.

FIGS. 1A-1D provides schematic representations of reaction mechanisms of fused ring azo and diaza compounds for phototherapy methods, wherein photoactivation generates reactive species.

FIGS. 2A and 2B provide examples of synthetic schemes for derivatizing fused ring diaza photosensitizers of the invention. FIG. 2A provides a scheme for the addition of Br to unsaturated ring B of the fused ring diaza compound having formula (FX33) resulting in synthesis of a compound having formula (FX40). FIG. 2B provides a synthetic scheme for attaching a targeting peptide to a fused-ring diaza compound of the invention.

FIG. 3 is a bar graph illustrating leukemia cell viability results for control conditions (DMSO, electromagnetic radiation and no photosensitzer) wherein cells were exposed to electromagnetic radiation in presence of dimethyl sulfoxide.

FIG. 4 is a bar graph illustrating first leukemia cell viability results for test conditions wherein cells were exposed to electromagnetic radiation in presence of a fused ring diaza compound having formula (FX33).

### DETAILED DESCRIPTION

Referring to the drawings, like numerals indicate like elements and the same number appearing in more than one drawing refers to the same element. In general the terms and phrases used herein have their art-recognized meaning, which can be found by reference to standard texts, journal references and contexts known to those skilled in the art. The following definitions are provided to clarify their specific use in the context of the invention.

“Optical agent” generally refers to compounds, compositions, preparations, and/or formulations that absorb, emit, or scatter electromagnetic radiation of wavelength, generally in the range of 350-1300 nanometers, within a biologically relevant environment or condition. In some embodiments, optical agents of the invention, when excited by electromagnetic radiation, undergo emission via fluorescence or phosphorescence pathways. These pathways are useful for diagnostic imaging, visualization, or organ function monitoring. Compounds belonging to this class are commonly referred to as ‘optical imaging agents’ or ‘optical contrast agents.’ In some other embodiments, optical agents of the invention absorb electromagnetic radiation and undergo photochemical reactions such as photofragmentation of one or more photolabile bonds to generate reactive species such as nitrenes, carbene, free radicals, ions, excited species, etc. This process is useful for a wide range of phototherapy applications, for example in the treatment of tumors or other lesions. Compounds belonging to this class are commonly referred to as ‘photosensitizers.’ The term “photosensitizer” refers to a phototherapeutic agent or a component thereof providing for photoactivation, for example, photoactivation resulting in generation of reactive species that locally kill, injury, inactivate or otherwise degrade cells (e.g., cancer cells, tumor cells, non-cancer cells, etc.). Photosensitizers of some embodiments undergo photoactivation that initiates bond cleavage reactions, such as photolysis and/or nitrogen extrusion reactions, thereby generating reactive species capable of causing localized cell death or injury. Optical agents include Type 1 and Type 2 phototherapeutic agents.

Optical agents of the present invention can contain fluorophores. The term “fluorophore” generally refers to a component or moiety of a molecule which causes a molecule to be fluorescent. Fluorophores can be functional groups in a molecule which absorb electromagnetic radiation of first specific wavelengths and re-emit energy at second specific wavelengths. The amount and wavelengths of the emitted electromagnetic radiation depend on both the fluorophore and the chemical environment of the fluorophore.

Compounds and compositions of the invention provide optical agents including photosensitizers, phototherapeutic agents, imaging agents, dyes, and detectable agents; and conjugates, complexes, and derivatives thereof. Optical agents of the invention include fused ring azo or diaza compounds that undergo C—N, N—N and/or N═N bond cleavage reactions upon exposure to electromagnetic radiation having wavelengths selected over the range of 350 to 1300 nm, optionally 350 to 900 nm. Some optical agents of the invention provide detectable agents that can be administered to a subject and subsequently detected using a variety of optical techniques, including optical imaging, visualization, and one-, two-, three- and point optical detection.

Optical agents include, but are not limited to, phototherapeutic agents (Type 1 and 2), photosensitizers, imaging agents, dyes, detectable agents, photosensitizer agents, photoactivators, and photoreactive agents; and conjugates, complexes, and derivatives thereof.

“Phototherapy procedure” refers to a therapeutic procedure involving administration of a phototherapeutic agent to a patient followed by subsequent excitation by exposure to applied electromagnetic radiation, such as electromagnetic radiation having wavelengths in the visible and/or near IR region of the electromagnetic spectrum such as wavelengths in the range of 350-1300 nanometers, so as to generate a therapeutically effective amount of excited phototherapeutic agent. Phototherapy includes, but is not limited to, photodynamic therapy. As used herein phototherapy includes procedures involving administration of Type 1 and/or Type 2 phototherapeutic agents, optionally further including administration of one or more additional therapeutic agents.

As used herein, “targeting ligand” (abbreviated as Bm) refers to a chemical group and/or substituent having functionality for targeting a compound of any one of formula (FX1)-(FX40) to an anatomical and/or physiological site of a patient, such as a selected cell, tissue or organ. For some embodiments, a targeting ligand is characterized as a ligand that selectively or preferentially binds to a specific biological site(s) (e.g., enzymes, receptors, etc.) and/or biological surface(s) (e.g., membranes, fibrous networks, etc.). In an embodiment, the invention provides compounds having any one of formula (FX1)-(FX40), wherein Bm is an amino acid, or a polypeptide comprising 2 to 30 amino acid units. In an embodiment, the invention provides compounds having any one of formula (FX1)-(FX40), wherein Bm is a mono- or polysaccharide comprising 1 to 50 carbohydrate units. In an embodiment, the invention provides compounds having any one of formula (FX1)-(FX40), wherein Bm is a mono-, oligo- or poly-nucleotide comprising 1 to 50 nucleic acid units. In an embodiment, the invention provides compounds having any one of formula (FX1)-(FX40), wherein Bm is a protein, an enzyme, a carbohydrate, a peptidomimetic, a glycomimetic, a glycopeptide, a glycoprotein, a lipid, an antibody (polyclonal or monoclonal), or fragment thereof. In an embodiment, the invention provides compounds having any one of formula (FX1)-(FX40), wherein Bm is an aptamer. In an embodiment, the invention provides compounds having any one of formula (FX1)-(FX40), wherein Bm is a drug, a hormone, steriod or a receptor. In some embodiments, each occurrence of Bm in the compounds of (FX1)-(FX40) is independently a monoclonal antibody, a polyclonal antibody, a metal complex, an albumin, or an inclusion compound such as a cyclodextrin. In some embodiments, each occurrence of Bm in the compounds of (FX1)-(FX40) is independently integrin, selectin, vascular endothelial growth factor, fibrin, tissue plasminogen, thrombin, LDL, HDL, Sialyl LewisX or a mimic thereof, or an atherosclerotic plaque binding molecule. In formula (FX34)-(FX40), the shown “Biomolecule” component may be a targeting ligand (Bm).

Specific examples of targeting ligands include steroid hormones for the treatment of breast and prostate lesions, whole or fragmented somatostatin, bombesin, and neurotensin receptor binding molecules for the treatment of neuroendocrine tumors, whole or fragmented cholecystekinin receptor binding molecules for the treatment of lung cancer, whole or fragmented heat sensitive bacterioendotoxin (ST) receptor and carcinoembryonic antigen (CEA) binding molecules for the treatment of colorectal cancer, dihydroxyindolecarboxylic acid and other melanin producing biosynthetic intermediates for melanoma, whole or fragmented integrin receptor and atherosclerotic plaque binding molecules for the treatment of vascular diseases, and whole or fragmented amyloid plaque binding molecules for the treatment of brain lesions. In some embodiments, Bm, if present, is selected from heat-sensitive bacterioendotoxin receptor binding peptide, carcinoembryonic antigen antibody (anti-CEA), bombesin receptor binding peptide, neurotensin receptor binding peptide, cholecystekinin receptor binding peptide, somastatin receptor binding peptide, ST receptor binding peptide, neurotensin receptor binding peptide, steriod receptor binding peptide, carbohydrate receptor binding peptide or estrogen. In another embodiment Bm, if present, is a ST enterotoxin or fragment thereof. In some embodiments, Bm, if present, is selected from octreotide and octreotate peptides. In an other embodiment Bm, if present, is a synthetic polymer. Examples of synthetic polymers useful for some applications include polyaminoacids, polyols, polyamines, polyacids, oligonucleotides, aborols, dendrimers, and aptamers.

“Peptidomimetic” refers to a small molecule having activity that resembles that of a polypeptide. Morphine, for example, is a peptidomimetic of endorphin peptide.

“Target tissue” refers to tissue of a subject to which an optical agent is administered or otherwise contacted, for example during a biomedical procedure such as an optical imaging, phototherapy or visualization procedure. Target tissue may be contacted with an optical agent of the invention under in vivo conditions or ex vivo conditions. Target tissues in some methods of the invention include cancerous tissue, cancer cells, precancerous tissue, a tumor, a lesion, a site of inflammation, or vasculature tissue. Target tissue in some methods of the invention includes a melanoma cell, a breast lesion, a prostate lesion, a lung cancer cell, a colorectal cancer cell, an atherosclerotic plaque, a brain lesion, a blood vessel lesion, a lung lesion, a heart lesion, a throat lesion, an ear lesion, a rectal lesion, a bladder lesion, a stomach lesion, an intestinal lesion, an esophagus lesion, a liver lesion, a pancreatic lesion, and a solid tumor. Target tissue in some embodiments refers to a selected organ of the subject or component thereof, such as lung, heart, brain, stomach, liver, kidneys, gallbladder, pancreas, intestines, rectum, skin, colon, prostate, ovaries, breast, bladder, blood vessel, throat, ear, or esophagus.

The term “inflammation” generally refers to a biological response of vascular tissues to harmful stimuli, such as pathogens, damaged cells, irritants, etc. Inflammation can be either acute or chronic. Acute inflammation is an initial response of the body to harmful stimuli and can be achieved by the increased movement of plasma and leukocytes from the blood into injured tissues. An inflammatory response can involve the local vascular system, the immune system, and/or various cells within the injured tissue. Prolonged inflammation, referred to as chronic inflammation, can lead to a progressive shift in the type of cells which are present at the site of inflammation can be characterized by simultaneous destruction and healing of the tissue from the inflammatory process.

As used herein, “tumor-specific agent” refers to an entity, such as an optical agent, that preferentially accumulates in a tumor at a higher level than normal tissue regardless of the particular mechanism of uptake in the tumors, for example, either receptor mediated or enhance permeability and retention (EPR). Optical agents of the invention include tumor-specific agents, including tumor specific phototherapy agents, for example having a targeting ligand providing specificity in the administration, delivery and/or binding to tumor tissue.

The terms “peptide” and “polypeptide” are used synonymously in the present description, and refer to a class of compounds composed of amino acid residues chemically bonded together by amide bonds (or peptide bonds). Peptides and polypetides are polymeric compounds comprising at least two amino acid residues or modified amino acid residues. Modifications can be naturally occurring or non-naturally occurring, such as modifications generated by chemical synthesis. Modifications to amino acids in peptides include, but are not limited to, phosphorylation, glycosylation, lipidation, prenylation, sulfonation, hydroxylation, acetylation, methionine oxidation, alkylation, acylation, carbamylation, iodination and the addition of cofactors. Peptides include proteins and further include compositions generated by degradation of proteins, for example by proteolyic digestion. Peptides and polypeptides may be generated by substantially complete digestion or by partial digestion of proteins. Polypeptide targeting ligands include, for example, polypeptides comprising 1 to 100 amino acid units, optionally for some embodiments 1 to 50 amino acid units and, optionally for some embodiments 1 to 20 amino acid units.

“Protein” refers to a class of compounds comprising one or more polypeptide chains and/or modified polypeptide chains. Proteins may be modified by naturally occurring processes such as post-translational modifications or co-translational modifications. Exemplary post-translational modifications or co-translational modifications include, but are not limited to, phosphorylation, glycosylation, lipidation, prenylation, sulfonation, hydroxylation, acetylation, methionine oxidation, the addition of cofactors, proteolysis, and assembly of proteins into macromolecular complexes. Modification of proteins may also include non-naturally occurring derivatives, analogues and functional mimetics generated by chemical synthesis. Exemplary derivatives include chemical modifications such as alkylation, acylation, carbamylation, iodination or any modification that derivatives the protein.

The terms “oligonucleotide” and “polynucleotide” refers to a class of compounds composed of nucleic acid residues chemically bonded together. Targeting ligands of the invention include oligonucleotides and polynucleotides comprising a plurality of nucleic acid residues, such as DNA or RNA residues, and/or modified nucleic acid residues. Modifications to nucleic acid residues can be naturally occurring or non-naturally occurring, such as modifications generated by chemical synthesis. Oligo- or poly-nucleotide targeting ligands include, for example, oligo- or poly-nucleotides comprising 1 to 100 nucleic acid units, optionally for some embodiments 1 to 50 nucleic acid units and, optionally for some embodiments 1 to 20 nucleic acid units.

The term “aptamer” refers to an oligo- or poly-nucleotide or polypeptide that binds to, or otherwise selectively associates with, a specific target molecule. For example, the invention provides optical agents having an aptamer targeting ligand that preferentially binds to proteins, peptides or other biomolecules expressed, or otherwise generated by, a target tissue, such as a tumor, precancerous tissue, site of inflammation or other lesion.

As used herein, “spacer moiety” refers to a component provided between the central fused ring core of some compounds of the invention and any of R¹-R⁷. In some embodiments, any one of L¹-L⁷ in formulas (FX1)-(FX28) is a spacer moiety. Spacer moieties useful for some embodiments are provided between any of R¹-R⁷ and the central fused ring core to enhance the overall chemical, optical, physical and/or pharmacokinetic properties of an optical agent of the invention. Useful spacer moieties for compounds of the invention having formulas (FX1)-(FX28) include C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—, wherein each of a and b is independently selected from the range of 1 to 100, optionally selected from the range of 1 to 30 and optionally selected from the range of 1 to 10. The invention includes compounds having formulas (FX1)-(FX40) that do not have a spacer moiety.

As used herein, “attaching moiety” refers to a component provided to attach any of R¹-R⁷ directly or indirectly to the central fused ring core in compounds of the invention. In some embodiments, any one of W¹-W⁷ in formulas (FX1)-(FX28) is an attaching moiety. Attaching moieties may connect to the central fused ring core directly or may connect to the central fused ring core via a spacer moiety. Attaching moieties in some embodiments provide a means of derivatizing the central fused ring core so as to provide optical agents having useful overall chemical, optical, physical and/or pharmacokinetic properties, including targeting and molecular recognition functionality. Attaching moieties useful in the invention include, but are not limited to, a single bond, —(CH₂)ₙ—, —(HCCH)ₙ—, —O—, —S—, —SO—, —SO₂—, —SO₃—, —OSO₂—, —NR¹⁴—, —CO—, —COO—, —OCO—, —OCOO—, —CONR¹⁵—, —NR¹⁶CO—, —OCONR¹⁷—, —NR¹⁸COO—, —NR¹⁹CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ—, —OCO(CH₂)ₙ—, —OCOO(CH₂)ₙ—, —CONR²⁴(CH₂)ₙ—, —CONR²⁵(CH₂)ₙ—, —NR²⁶CO(CH₂)ₙ—, —OCONR²⁷(CH₂)ₙ—, —NR²⁸COO(CH₂)ₙ—, —NR²⁹CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—, wherein each n is independently selected from the range of 1 to 10 and wherein R¹⁴-R³⁶ are defined as described in connection with formula (FX1).

As used herein, an “electron withdrawing group” (abbreviated as “EWG”) refers to a chemical group that draws electrons or electron density from a center, such as any of rings A-G of the fused ring azo and diaza compounds of the invention. In some embodiments, the electron withdrawing group(s) are independently selected from cyano (—CN), carbonyl (—CO), carboxylates (—CO₂R⁴⁰), halo (—F, —Cl, —Br, —I, —At), carbamates (—CONR⁵²R⁵³), acyl (—COR⁵⁴), nitro (—NO₂), sulfinyl (—SOR⁴¹), sulfonyl (—SO₂R⁵⁵), —SO₂OR⁴³, and —PO₃R⁵⁶R⁵⁷; wherein in the context of this description, R⁴⁰-R⁵⁷ are independently selected to enhance biological and/or physiochemical properties of the optical agents of the invention. In some instances, R⁵⁵-R⁶² are independently selected from any one of a hydrogen atom, an anionic functional group (e.g., carboxylate, sulfonate, sulfate, phosphonate and phosphate) and a hydrophilic functional group (e.g., hydroxyl, carboxyl, sulfonyl, sulfonato and phosphonato). In other instances, R⁴⁰-R⁵⁷ are independently selected form hydrogen, C₁₋₁₀ alkyl, aryl, heteroaryl, —(CH₂)ₐOH, —(CH₂)ₐCO₂H, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃⁼, —(CH₂)ₐOPO₃H₂, —(CH₂)ₐOPO₃H⁻ and —(CH₂)ₐOPO₃; wherein a is an integer from 1 to 10. In one example of this embodiment, the EWG(s) are independently selected from —CN, halo, C₁-C₁₀ acyl, —CO₂R⁴⁰, —SOR⁴¹, —OSR⁴², —SO₂OR⁴³, —CONR⁵²R⁵³; —COR⁵⁴; —NO₂, —SO₂R⁵⁵, —SO₂NR⁵⁸R⁵⁹, and —PO₃R⁵⁶R⁵⁷, wherein R⁴⁰-R⁵⁹ are as described in the context of compounds of formula (FX1). In an embodiment, an EWG is located at the terminus of a substituent arm of any of rings A-G of the fused ring azo and diaza compounds of formulas (FX1)-(FX40).

As used herein, an “electron donating group” (abbreviated as “EDG”) refers to a chemical group that releases electrons or electron density to a center, such any of rings A-G of the fused ring azo and diaza compounds of the invention. In some embodiments, the electron donating group(s) are independently selected from C₁-C₁₀ alkyl, C₅-C₁₀ aryl, —(CH₂)ₐOH, —OR⁴⁶, —SR⁴⁷, —NR48R⁴⁹, —N(R⁵⁰ )COR⁵¹, and —P(R⁷¹); wherein in the context of this description, R⁴⁶-R⁷¹ are independently selected to enhance biological and/or physiochemical properties of the optical agents of the invention and wherein a is selected from the range of 1 to 10. In some instances, R⁴⁶-R⁷¹ are independently selected from any one of a hydrogen atom, an anionic functional group (e.g., carboxylate, sulfonate, sulfate, phosphonate and phosphate) and a hydrophilic functional group (e.g., hydroxyl, carboxyl, sulfonyl, sulfonato and phosphonato). In other instances, R⁴⁶-R⁷¹ are independently selected from hydrogen, C₁₋₁₀ alkyl, aryl, heteroaryl, —(CH₂)ₐOH, —(CH₂)ₐCO₂H, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃⁼, —(CH₂)ₐOPO₃H₂, —(CH₂)ₐOPO₃H⁻ and —(CH₂)ₐOPO₃ where a is an integer from 1 to 10. In one example of this embodiment, the EDG(s) are independently C₁-C₁₀ alkyl, —NR⁴⁸R⁴⁹, —OR⁴⁶, —NR⁵⁰COR⁵¹, or —SR⁴⁷, wherein R⁴⁶-R⁵¹ are as described in the context of compounds of formulas (FX1). In an embodiment, an EDG is located at the terminus of a substituent arm of any of rings A-G of the fused ring azo and diaza compounds of formulas (FX1)-(FX40) invention.

When used herein, the terms “diagnosis”, “diagnostic” and other root word derivatives are as understood in the art and are further intended to include a general monitoring, characterizing and/or identifying a state of health or disease. The term is meant to encompass the concept of prognosis. For example, the diagnosis of cancer can include an initial determination and/or one or more subsequent assessments regardless of the outcome of a previous finding. The term does not necessarily imply a defined level of certainty regarding the prediction of a particular status or outcome.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids.

As defined herein, “administering” means that a compound or formulation thereof of the invention, such as an optical agent, is provided to a patient or subject, for example in a therapeutically effective amount. The invention includes methods for a biomedical procedure wherein a therapeutically or diagnostically effective amount of a compound having any one of formulas (FX1)-(FX40) is administered to a patient in need of treatment, for example to a patient undergoing treatment for a diagnosed diseased state including cancer and vascular diseases. Administering may be carried out by a range of techniques known in the art including intravenous, intraperitoneal or subcutaneous injection or infusion, oral administration, transdermal absorption through the skin, or by inhalation.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and may also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Aryl groups include groups having one or more 5-, 6- or 7-member aromatic or heterocyclic aromatic rings. Aryl groups can contain one or more fused aromatic rings. Heterocyclic aromatic rings can include one or more N, O, or S atoms in the ring. Heterocyclic aromatic rings can include those with one, two or three N, those with one or two O, and those with one or two 5, or combinations of one or two or three N, O or S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following benzene, naphthalene, naphthoquinone, diphenylmethane, fluorene, anthracene, anthraquinone, phenanthrene, tetracene, naphthacenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic radical, including monovalent, divalent and polyvalent radicals, of the aromatic and heterocyclic aromatic groups listed above provided in a covalently bonded configuration in the compounds of the invention. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl having one or more hydrogens replaced with one or more fluorine atoms, chlorine atoms, bromine atoms and/or iodine atoms.

Optional substitution of any alkyl, alkenyl and aryl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for alkyl, alkenyl and aryl groups include among others:

—COOR where R is a hydrogen or an alkyl group or an aryl group and more specifically where R is methyl, ethyl, propyl, butyl, or phenyl groups all of which are optionally substituted;

—COR where R is a hydrogen, or an alkyl group or an aryl groups and more specifically where R is methyl, ethyl, propyl, butyl, or phenyl groups all of which groups are optionally substituted;

—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—N(R)₂ where each R, independently of each other R, is a hydrogen, or an alkyl group, acyl group or an aryl group and more specifically where R is methyl, ethyl, propyl, butyl, or phenyl or acetyl groups all of which are optionally substituted; or R and R can form a ring which may contain one or more double bonds.

—SR, —SO₂R, or —SOR where R is an alkyl group or an aryl groups and more specifically where R is methyl, ethyl, propyl, butyl, phenyl groups all of which are optionally substituted; for —SR, R can be hydrogen;

—OCOOR where R is an alkyl group or an aryl groups;

—SO₂N(R)₂ where R is a hydrogen, an alkyl group, or an aryl group and R and R can form a ring;

—OR where R is H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted.

As used herein, the term “alkylene” refers to a divalent radical derived from an alkyl group as defined herein. Alkylene groups in some embodiments function as attaching and/or spacer groups in the present compositions. Compounds of the invention include substituted and unsubstituted C₁-C₂₀ alkylene, C₁-C₁₀ alkylene and C₁-C₅ alkylene groups.

As used herein, the term “cycloalkylene” refers to a divalent radical derived from a cycloalkyl group as defined herein. Cycloalkylene groups in some embodiments function as attaching and/or spacer groups in the present compositions. Compounds of the invention include substituted and unsubstituted C₁-C₂₀ cycloalkylene, C₁-C₁₀ cycloalkylene and C₁-C₅ cycloalkylene groups.

As used herein, the term “alkenylene” refers to a divalent radical derived from an alkenyl group as defined herein. Alkenylene groups in some embodiments function as attaching and/or spacer groups in the present compositions. Compounds of the invention include substituted and unsubstituted C₁-C₂₀ alkenylene, C₁-C₁₀ alkenylene and C₁-C₅ alkenylene groups.

As used herein, the term “cylcoalkenylene” refers to a divalent radical derived from a cylcoalkenyl group as defined herein. Cycloalkenylene groups in some embodiments function as attaching and/or spacer groups in the present compositions. Compounds of the invention include substituted and unsubstituted C₁-C₂₀ cylcoalkenylene, C₁-C₁₀ cylcoalkenylene and C₁-C₅ cylcoalkenylene groups.

As used herein, the term “alkynylene” refers to a divalent radical derived from an alkynyl group as defined herein. Alkynylene groups in some embodiments function as attaching and/or spacer groups in the present compositions. Compounds of the invention include substituted and unsubstituted C₁-C₂₀ alkynylene, C₁-C₁₀ alkynylene and C₁-C₅ alkynylene groups.

As used herein, the term “halo” refers to a halogen group such as a fluoro (—F), chloro (—Cl), bromo (—Br), iodo (—I) or astato (—At).

As used herein, the term “azo” refers to a group having at least one —N═N— moiety. Azo groups include cyclic and acyclic groups having an —N═N— moiety, for example: (i) aryl-azo groups having an —N═N— moiety directly or indirectly linked to one or more carbocyclic or heterocyclic aromatic rings of a C₅-C₂₀ aryl, (ii) alkyl-azo groups having an —N═N— moiety directly or indirectly linked to a C₁-C₂₀ alkyl group and (iii) alkylaryl-azo groups having an —N═N— moiety directly or indirectly linked to a C₁-C₂₀ alkyl group and one or more carbocyclic or heterocyclic aromatic rings of a C₅-C₂₀ aryl. In an embodiment, for example, an azo group of a compound of the invention includes a cyclic group having an intra-ring —N═N— group. In an embodiment, for example, an azo group of a compound of the invention includes a cyclic group wherein a carbon-carbon bond in a carbocyclic or heterocyclic ring is replaced with a nitrogen-nitrogen double bond (i.e. N═N). In an embodiment, for example, an azo compound of the invention includes a fused ring structure comprising one or more aromatic groups and one or more alicyclic groups, wherein a carbon-carbon bond in a carbocyclic or heterocyclic ring of the alicyclic group is replaced with a nitrogen-nitrogen double bond (i.e. N═N).

As used herein, the term “diaza” refers to a group having at least one —(R)N—N(R)— moiety or ═N—N(R)— moiety. Diaza groups include cyclic and acyclic groups having a —(R)N—N(R)— moiety or ═N—N(R)— moiety, for example: (i) aryl-diaza groups having an a —(R)N—N(R)— moiety or ═N—N(R)— moiety directly or indirectly linked to one or more carbocyclic or heterocyclic aromatic rings of a C₅-C₂₀ aryl, (ii) alkyl-diaza groups having an a —(R)N—N(R)— moiety or ═N—N(R)— moiety directly or indirectly linked to a C₁-C₂₀ alkyl group and (iii) alkylaryl-diaza groups having a —(R)N—N(R)— moiety or ═N—N(R)— moiety directly or indirectly linked to a C₁-C₂₀ alkyl group and one or more carbocyclic or heterocyclic aromatic rings of a C₅-C₂₀ aryl. In an embodiment, for example, a diaza group of a compound of the invention includes a cyclic group having an intra-ring —(R)N—N(R)— or ═N—N(R)— group. In an embodiment, for example, a diaza group of a compound of the invention includes a cyclic group wherein a carbon-carbon bond in a carbocyclic or heterocyclic ring is replaced with a nitrogen-nitrogen single bond (i.e. N—N). In an embodiment, for example, a diaza compound of the invention includes a fused ring structure comprising one or more aromatic groups and one or more alicyclic groups, wherein a carbon-carbon bond in a carbocyclic or heterocyclic ring of the alicyclic group is replaced with a nitrogen-nitrogen single bond (i.e. N—N).

The term “heterocyclic” refers to ring structures containing at least one other kind of atom, in addition to carbon, in the ring. Examples of such atoms include nitrogen, oxygen and sulfur. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term carbocyclic refers to ring structures containing only carbon atoms in the ring. Carbon atoms of carbocyclic rings may be bonded to a wide range of other atoms and function groups.

Alicyclic refers to a ring that is not an aromatic ring. Alicyclic rings include both carbocyclic and heterocyclic rings.

Alkoxyalkyl: As used herein, the term “alkoxyalkyl” refers to a substituent of the formula alkyl-O-alkyl.

Polyhydroxyalkyl: As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

Polyalkoxyalkyl: As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

As used herein, the term “luminescence” refers to the emission of electromagnetic radiation from excited electronic states of atoms or molecules. Luminescence generally refers to electromagnetic radiation emission, such as photoluminescence, chemiluminescence, and electrochemiluminescence, among others. In photoluminescence, including fluorescence and phosphorescence, the excited electronic state is created by the absorption of electromagnetic radiation. Luminescence detection involves detection of one or more properties of the luminescence or associated luminescence process. These properties may include intensity, excitation and/or emission spectrum, polarization, lifetime, and energy transfer, among others. These properties may also include time-independent (steady-state) and/or time-dependent (time-resolved) properties of the luminescence. Representative luminescence techniques include fluorescence intensity (FLINT), fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), and bioluminescence resonance energy transfer (BRET), among others. By way of example, when an optical agent is used in the present invention, it is desirable that the wavelength of non-ionizing radiation be such that it excites the optical agent. This excitation causes a bond of the molecule to break thus releasing an appropriate radical. This excitation may also cause the molecule to emit part of the absorbed energy at a different wavelength; such emission may be detected using fluorometric techniques as described above. One skilled in the art can readily determine the most appropriate treatment and optional detection technique based, at least in part, the specific phototherapeutic agent(s) administered and/or the particular use (e.g., tissue to be treated).

As used herein, the term “controlled-release component” refers to an agent that facilitates the controlled-release of a compound including, but not limited to, polymers, polymer matrices, gels, permeable membranes, liposomes, microspheres, or the like, or any combination thereof. Methods for producing compounds in combination with controlled-release components are known to those of skill in the art.

As used herein, the term “pharmaceutically acceptable” means approved by a regulatory agency of an appropriate federal or state government or listed in the U.S. Pharmacopoeia or other generally recognized pharmacopoeia for use in animals, and more particularly in humans or does not impart significant deleterious or undesirable effect on a subject to whom it is administered and in the context in which it is administered.

As is customary and well known in the art, hydrogen atoms in formulas (FX1)-(FX40) are not always explicitly shown, for example, hydrogen atoms bonded to the carbon atoms of aromatic and alicyclic rings are not always explicitly shown in formulas (FX1)-(FX40). The structures provided herein, for example in the context of the description of formulas (FX1)-(FX40), are intended to convey to one of reasonable skill in the art the chemical composition of compounds of the methods and compositions of the invention, and as will be understood by one of skill in the art, the structures provided do not indicate the specific bond angles between atoms of these compounds.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible. In addition, the compounds of this invention include all stereochemical isomers arising from the substitution of these compounds.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, -cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other pharmaceutically acceptable salts may be found, for example, in Stahl et al., Handbook of Pharmaceutical Salts: Properties, Selection, and Use, Wiley-VCH; Verlag Helvetica Chimica Acta, Zurich, 2002. (ISBN 3-906390-26-8). Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The compounds of this invention may contain one or more chiral centers. Accordingly, this invention is intended to include racemic mixtures, diasteromers, enantiomers, tautomers and mixtures enriched in one or more steroisomer. The scope of the invention as described and claimed encompasses the racemic forms of the compounds as well as the individual enantiomers and non-racemic mixtures thereof.

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the invention which will be limited only by the appended claims.

In certain embodiments, the invention encompasses administering optical agents useful in the invention to a patient or subject. A “patient” or “subject”, used equivalently herein, refers to an animal. In particular, an animal refers to a mammal, preferably a human. The subject may either: (1) have a condition diagnosable, preventable and/or treatable by administration of an optical agent of the invention; or (2) is susceptible to a condition that is diagnosable, preventable and/or treatable by administering an optical agent of this invention.

Compositions of the invention includes formulations and preparations comprising one or more of the present optical agents provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

In an embodiment, the invention provides a pharmaceutical formulation having an active ingredient comprising a composition of the invention, such as a compound of any one of formulas (FX1)-(FX40). In an embodiment, the invention provides a method of synthesizing a composition of the invention or a pharmaceutical formulation thereof, such as a compound of any one of formulas (FX1)-(FX40). In an embodiment, a pharmaceutical formulation comprises one or more excipients, carriers, diluents, and/or other components as would be understood in the art. Preferably, the components meet the standards of the National Formulary (“NF”), United States Pharmacopoeia (“USP”; United States Pharmacopeial Convention Inc., Rockville, Md.), or Handbook of Pharmaceutical Manufacturing Formulations (Sarfaraz K. Niazi, all volumes, ISBN: 9780849317521, ISBN 10: 0849317525; CRC Press, 2004). See, e.g., United States Pharmacopeia and National Formulary (USP 30-NF 25), Rockville, Md.: United States Pharmacopeial Convention; 2007; and 2008, and each of any earlier editions; The Handbook of Pharmaceutical Excipients, published jointly by the American Pharmacists Association and the Pharmaceutical Press (Pharmaceutical Press (2005) (ISBN-10: 0853696187, ISBN-13: 978-0853696186); Merck Index, Merck & Co., Rahway, N.J.; and Gilman et al., (eds) (1996); Goodman and Gilman's: The Pharmacological Bases of Therapeutics, 8th Ed., Pergamon Press. In embodiments, the formulation base of the formulations of the invention comprises physiologically acceptable excipients, namely, at least one binder and optionally other physiologically acceptable excipients. Physiologically acceptable excipients are those known to be usable in the pharmaceutical technology sectors and adjacent areas, particularly, those listed in relevant pharmacopeias (e.g. DAB, Ph. Eur., BP, NF, USP), as well as other excipients whose properties do not impair a physiological use.

In an embodiment, an effective amount of a composition of the invention is a therapeutically effective amount. As used herein, the phrase “therapeutically effective” qualifies the amount of compound administered in the therapy. This amount achieves the goal of ameliorating, suppressing, eradicating, preventing, reducing the risk of, or delaying the onset of a targeted condition. In an embodiment, an effective amount of a composition of the invention is a diagnostically effective amount. As used herein, the phrase “diagnostically effective” qualifies the amount of compound administered in diagnosis, for example of a disease state or other pathological condition. The amount achieves the goal of being detectable while avoiding adverse side effects found with higher doses. In an embodiment, an active ingredient or other component is included in a therapeutically acceptable amount. In an embodiment, an active ingredient or other component is included in a diagnostically acceptable amount.

Variations on compositions including salts and ester forms of compounds: Compounds of this invention and compounds useful in the methods of this invention include those of the compounds and formula(s) described herein and pharmaceutically-acceptable salts and esters of those compounds. In embodiments, salts include any salts derived from the acids of the formulas herein which acceptable for use in human or veterinary applications. In embodiments, the term esters refers to hydrolyzable esters of compounds of the names and structural formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

In an embodiment, the invention provides a method for treating or diagnosing a medical condition comprising administering to a subject (e.g. patient) in need thereof, a therapeutically effective amount or diagnostically effective amount of a composition of the invention, such as a compound of any one of formulas (FX1)-(FX40). In an embodiment, the medical condition is cancer, or various other diseases, injuries, and disorders, including cardiovascular disorders such as atherosclerosis and vascular restenosis, inflammatory diseases, ophthalmic diseases and dermatological diseases.

In an embodiment, the invention provides a medicament which comprises a therapeutically effective amount of one or more compositions of the invention, such as a compound of any one of formulas (FX1)-(FX40). In an embodiment, the invention provides a medicament which comprises a diagnostically effective amount of one or more compositions of the invention. In an embodiment, the invention provides a method for making a medicament for treatment of a condition described herein. In an embodiment, the invention provides a method for making a medicament for diagnosis or aiding in the diagnosis of a condition described herein. In an embodiment, the invention provides the use of one or more compositions set forth herein for the making of a medicament.

Compounds of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alis, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds set forth herein.

In an embodiment, a composition of the invention is isolated or purified. In an embodiment, an isolated or purified compound may be at least partially isolated or purified as would be understood in the art.

The invention is further detailed in the following Examples, which are offered by way of illustration and are not intended to limit the scope of the invention in any manner.

### EXAMPLE 1

### Compounds for Phototherapy

### 1.a Type 1 Phototherapeutic Agents

The invention provides Type 1 phototherapeutic agents, including compositions, preparations and formulations, and methods of using and making Type 1 phototherapeutic agents. Type 1 phototherapeutic agents of the invention include compounds comprising a first ring A having an intra-ring azo or intra-ring diaza group, a second unsaturated ring B and a third aromatic ring, provided in a fused ring configuration wherein ring A is fused to both unsaturated ring B and aromatic ring C, and wherein unsaturated ring B and aromatic ring C are also fused to each other. Incorporation of unsaturated ring B in the fused ring configuration in some compounds may enhance stability prior to photoactivation and may extend conjugation in the fused ring azo or diaza compound, thereby allowing for photoactivation and internal energy transfer processes upon absorption of longer wavelength electromagnetic radiation as compared to analogs wherein unsaturated ring B is substituted with a saturated 6 membered ring. Fused ring A, B and C of some compositions of the invention functions as an aromatic antenna group for coupling energy from incident electromagnetic radiation into the phototherapeutic agent. In some phototherapeutic agents of the invention, energy coupled into the phototherapeutic agent is subsequently transferred to the surroundings to achieve a desired therapeutic outcome. Incorporation of an aromatic antenna group comprising fused ring A, B and C, and optionally additional carbocyclic and/or heterocyclic aromatic groups (e.g., rings D and E in formulas (FX21)-(FX28)), is useful in some embodiments for initiating cleavage of photolabile C—N, N—N and/or N═N bonds of ring A having the intra-ring azo or intra-ring diaza group upon absorption of electromagnetic radiation and subsequent internal energy transfer processes. In biomedical methods of the invention, cleavage of photolabile C—N, N—N and/or N═N bonds generates reactive species capable of causing localized tissue damage, such as cell death, inactivation or injury useful for phototherapy.

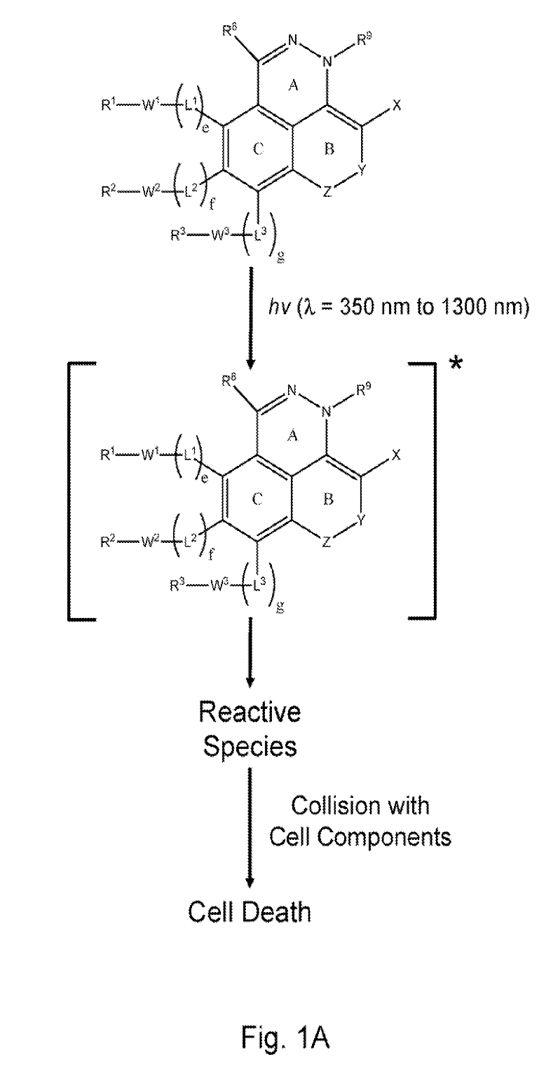

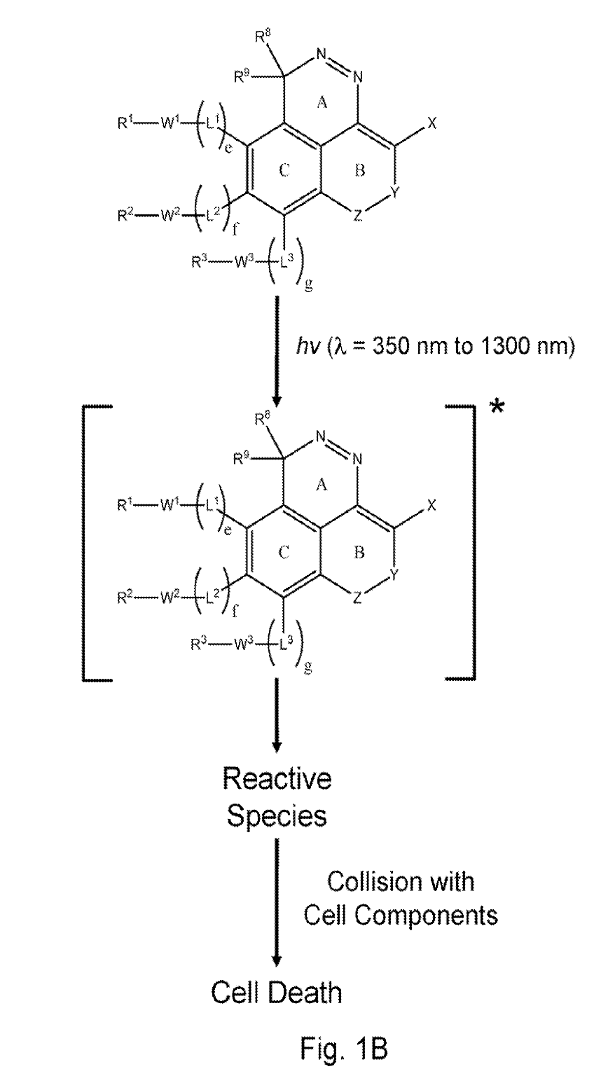

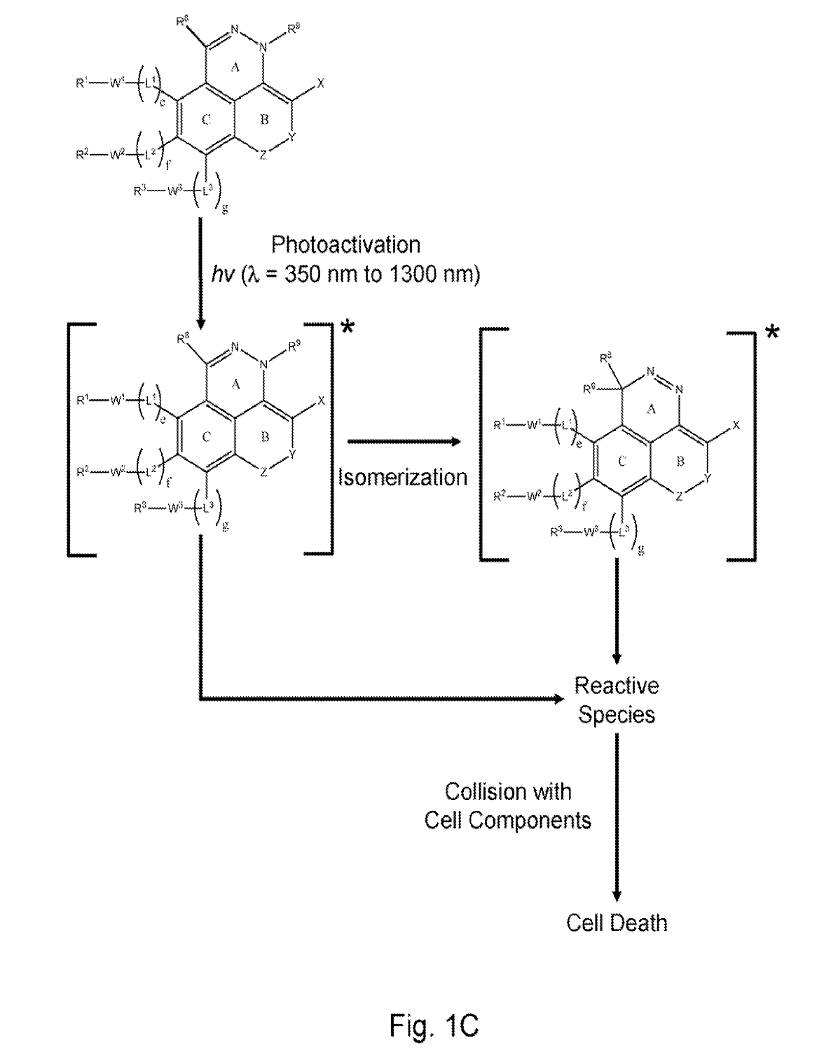

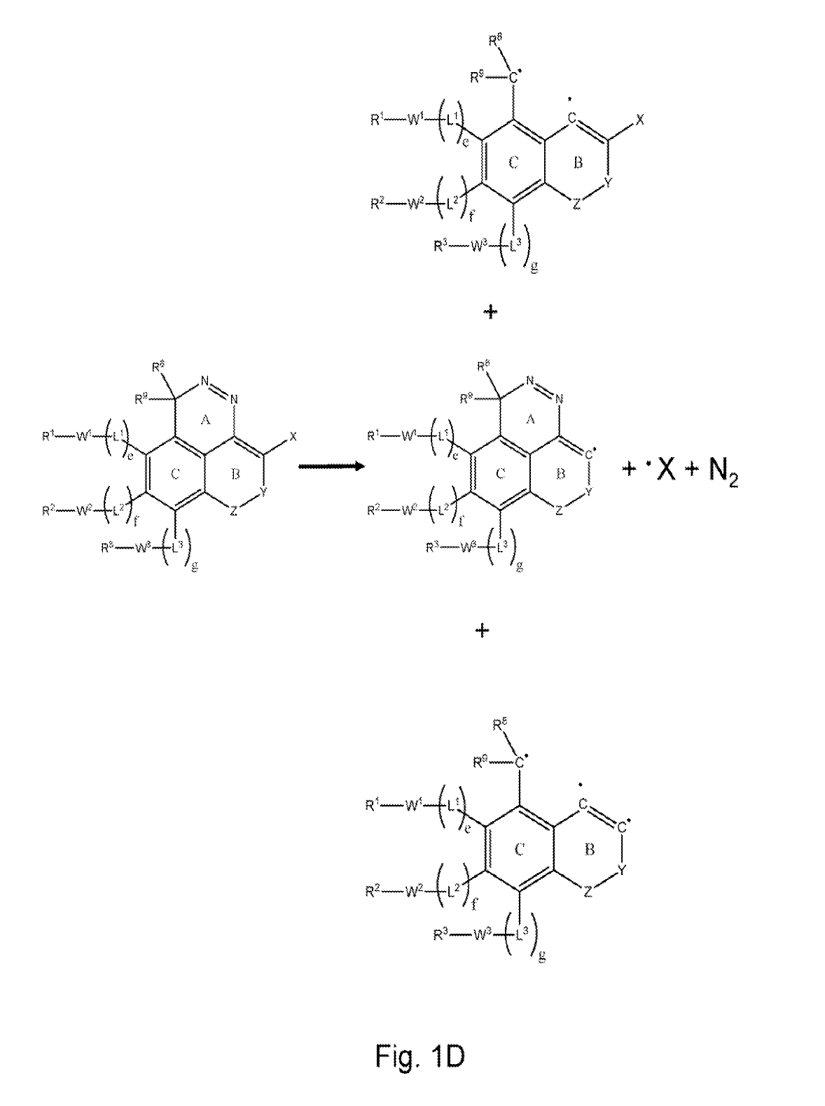

Some compounds of the invention operate through the Type 1 phototherapy mechanism as schematically illustrated in FIGS. 1A-1D wherein the photosensitizer is photoactivated upon exposure to electromagnetic radiation, thereby producing reactive species. FIGS. 1A and 1C provide schematic representations of reaction mechanisms for phototherapeutic agents having formula (FX3) and FIGS. 1B and 1D provide schematic representations of reaction mechanisms for phototherapeutic agents having formula (FX4). As schematically represented by the arrow and by in FIGS. 1A-1D, fused ring azo or diaza compounds of the invention are photoactivated by exposure to visible or near infrared electromagnetic radiation, for example electromagnetic radiation having wavelengths ranging from 350 nm to 1300 nm. Absorption of at least a portion of the applied electromagnetic radiation generates a therapeutically effective amount of photoactivated photosensitizer, which is schematically represented in FIGS. 1A-1D by the compound provided in brackets with an asterisk symbol (*). Activation of the phototherapeutic agent may occur via a single photon absorption process, a mulitphoton absorption process or a combination of a single photon absorption process and a mulitphoton absorption process. The activated photosensitizer subsequently undergoes processes, such as internal energy transfer, bond cleavage and/or extrusion processes, resulting in formation of reactive species capable of causing a desired therapeutic result. Reactive species generated by the compounds of the invention may include free radicals, intramolecular diradicals, ions, electrons, electrophiles, nitrene, vibrationally excited species, and translationally excited species.

As illustrated in FIG. 1C, excitation of a fused ring diaza photosensitizer having formula (FX3) in some embodiments may causes tautomerization so as to generate an excited state corresponding to the azo tautomer, which subsequently generates reactive species, for example via nitrogen extrusion. Alternatively, excitation of a fused ring azo photosensitizer having formula (FX3) in some embodiments may directly result in formation of reactive species, as also shown in FIG. 1C, for example via cleavage of C—N bond(s). As illustrated in FIG. 1D, excitation of a diaza photosensitizer having formula (FX4) in some embodiments may result in a number free radical reactive species involving a range of processes, including radical formation by opening ring A, formation of a radical by cleavage of the C—X bond, nitrogen extrusion and formation of radicals having a plurality of unpaired electrons. Although a nitrogen extrusion mechanism is exemplified in FIGS. 1C and 1D, compounds of the invention may generate reactive species via other pathways including other bond cleavage pathways (e.g. cleavage of the N═N bond, N—N bond, C—N bond or cleavage of C—X bond wherein X is a halo group) depending on the composition of the compound and wavelength of electromagnetic radiation used for excitation. As shown in FIG. 1D, photoactivation can beneficially provide a plurality of radicals that may be utilized to cause cell death. Another benefit of compounds of the invention is the double bond associated with the carbon to which X is attached. This carbon-carbon double bond may tend to provide increased stability (e.g., in vivo) to the compound at least prior to photolysis. In some embodiments, for example, unsaturated ring B having an intra-ring alkene group is provided to allow for photoexcitation at longer wavelengths (e.g., visible or NIR electromagnetic radiation). In some embodiments, the reactive species generated upon excitation of the photosensitizer collide, react with, or otherwise interact with cell components of a target organ or tissue class, thereby resulting in death, injury and/or damage to cells at the target tissue.

Type 1 phototherapeutic agents useful for certain phototherapy applications incorporate rings C-E of formula (FX1)-(FX40), including aromatic groups, heterocyclic aromatic groups, polycyclic aromatic groups and polycyclic heterocyclic aromatic groups, that absorb strongly in the visible and/or NIR region of the electromagnetic spectrum. Rings C-E provide effective photoactivation by electromagnetic radiation having wavelengths selected over the range of 300 nm to 1300 nm include, but are not limited to, a group corresponding to benzene, azulenes, aza-azulenes, anthracenes, pyrazines, pyridazines, quinolines, quinoxalines, courmarins, phenoxazines, phenothiazines, rhodamines, and the like. The invention further includes phototherapeutic agents having one or more rings C-E comprising aromatic group(s) and heterocyclic aromatic group(s) that are functionalized by incorporation of heteroatom ring members and/or substituents on the ring structure(s) providing excitation wavelength selection and/or tunability. In some embodiments, for example, rings C-E comprise one or more aromatic or heterocyclic aromatic groups, optionally provided in a fused ring configuration and/or having one or more electron donating and/or electron withdrawing groups provided as ring substituents for providing selected excitation characteristics, such as a selected absorption spectrum and/or strong absorption in the visible and/or NIR regions. Some phototherapeutic agents of the invention operate, at least in part, via the Type 2 process involving formation of excited state oxygen (¹O₂), and optionally contain a C₅-C₂₀ aryl that is a group corresponding to a cyanine, indocyanine, phenothiazine, and phthalocyanines.

Selection of R¹-R⁹ in the compounds of any one of formulas (FX1)-(FX40) establishes, at least in part, the physical, chemical, optical and/or pharmacokinetic properties of optical agents for the present compositions and methods. In some embodiments, for example R¹-R⁹ are selected to provide optical properties supporting and enabling use of these compositions in phototherapeutic methods, such as providing one or more of the following: (i) large extinction coefficients; (ii) strong absorption in the visible and/or infrared regions of the electromagnetic spectrum (e.g., 350 to 1300 nanometers, preferably for some applications 350-900 nanometers); and (iii) a large quantum yield for the production of reactive species, such as free radicals or ions, capable of causing photoactivation initiated tissue damage. Selection of the composition of R¹-R⁹ in the compounds of any one of formulas (FX1)-(FX40) may also be based, at least in part, on a number of pharmacokinetic and physical properties supporting effective delivery and clearance of the optical agents of the present methods and compositions. Such factors may include solubility, toxicity, immune response, biocompatibility, and bioclearance considerations. In some embodiments, any one of R¹-R⁹ in the compounds of any one of formulas (FX1)-(FX40) comprise a hydrophilic group, a lipophilic group, hydrophobic group, or an amphiphilic group. In an embodiment, at least one of R¹-R⁹ is a substituent comprising poly(ethylene glycol) (PEG; —(CH₂OCH₂)_{b}R⁶¹), or a derivative of PEG.

In an embodiment, a phototherapeutic agent of the invention incorporates aromatic groups and/or heterocyclic aromatic groups that are derivatized by the addition of at least one electron withdrawing group and at least one electron donating group bonded directly or indirectly to a carbon atom of the ring structure. In an embodiment, for example, one or more the electron withdrawing (EWG) and electron donating (EDG) group(s) are directly attached to the ring structure of the aromatic group. In another embodiment, EWG and EDG are indirectly attached to the to the ring structure of the aromatic group through an unsaturated spacer that is in conjugation with the double bonds of a C₅-C₃₀ aryl group. Electron donating and withdrawing groups in these dye compositions may be positioned ortho, meta or para to each other with respect to the to the ring structure of the aromatic group. In some embodiments, for example, two electron withdrawing groups are positioned para to each other on the ring structure of the aromatic group and two electron donating groups are positioned para to each other on the ring structure of the aromatic group. In some embodiments, electron withdrawing groups and electron donating groups are positioned so as to make the overall compound symmetrical.

Optical agents of the invention support a broad therapeutic platform useful for a variety of in vivo phototherapy procedures, for example for the treatment of cancer, stenosis, inflammation, infection and arthritis. Optical agents of the invention are optionally multifunctional agents capable of providing a useful combination of photodiagnostic, phototherapeutic, molecular recognition and/or targeting functionality. In an embodiment, for example, a dye component is incorporated into the phototherapeutic agent of the present compositions for imparting useful optical functionality, for example by functioning as an optical absorber, chromophore, and/or fluorophore. This functionality is useful for targeted administration and excitation of the therapeutic agent. Optionally, optical agents of the invention further comprise a targeting component, such as a targeting ligand. In an embodiment, for example, an optical agent of the invention comprises a targeting ligand integrated with a photosensitizer component to access enhanced administration, delivery and photoactivation functionality for phototherapy therapy. Optical agents and bioconjugates thereof are provided having one or more targeting ligands covalently bonded to or noncovalently associated with the phototherapeutic agents of the invention, thereby providing specificity for administering, targeting, delivery and/or localizing an optical agent to a specific biological environment, such as a target tissue such as a specific organ, tissue, cell type or tumor site.

In the compounds of any one of formulas (FX1)-(FX40), Bm is a targeting ligand, optionally providing molecular recognition functionality. In some embodiments, the targeting ligand is a particular region of the compound that is recognized by, and binds to, a target site on the organ, tissue, tumor or cell. Targeting ligands are often, but not always, associated with biomolecules or fragments thereof which include hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Specific examples of biomolecules include steroid hormones for the treatment of breast and prostate lesions; somatostatin receptor binding molecules, bombesin receptor binding molecules, and neurotensin receptor binding molecules for the treatment of neuroendocrine tumors, cholecystekinin receptor binding molecules for the treatment of lung cancer; heat sensitive bacterioendotoxin (ST) receptor binding molecules and carcinoembryonic antigen (CEA) binding molecules for the treatment of colorectal cancer, dihydroxyindolecarboxylic acid and other melanin producing biosynthetic intermediates for melanoma, integrin receptor and atherosclerotic plaque binding molecules for the treatment of vascular diseases, amyloid plaque binding molecules for the treatment of brain lesions, cholecystokinin (CCK) receptor binding molecules, steroid receptor binding molecules, carbohydrate receptor binding molecules, dihydroxyindole-2-carboxylic acid, and combinations thereof. Targeting ligands for use in the invention may also include synthetic polymers. Examples of synthetic polymers include polyaminoacids, polyols, polyamines, polyacids, oligonucleotides, aborols, dendrimers, and aptamers. Still other examples of appropriate targeting ligands may include integrin, selectin, vascular endothelial growth factor, fibrin, tissue plasminogen activator, thrombin, LDL, HDL, Sialyl LewisX and its mimics, and atherosclerotic plaque binding molecules.

Coupling of phototherapeutic and/or diagnostic agents to biomolecules can be accomplished by methods well known in the art as disclosed in Hnatowich et al., Radioactive Labeling of Antibody. A simple and efficient method. Science, 1983, 220, 613-615; A. Pelegrin et al., Photoimmunodiagnosis with antibody-fluorescein conjugates: in vitro and in vivo preclinical studies. Journal of Cellular Pharmacology, 1992, 3, 141-145; and U.S. Pat. No. 5,714,342, each of which are expressly incorporated by reference herein in their entirety. Successful specific targeting of fluorescent dyes to tumors using antibodies and peptides for diagnostic imaging of tumors has been demonstrated, for example, S. A. Achilefu et al., Novel receptor-targeted fluorescent contrast agents for in vivo tumor imaging, Investigative Radiology, 2000, 35(8), 479-485; B. Ballou et al., Tumor labeling in vivo using cyanine-conjugated monoclonal antibodies, Cancer immunology and Immunotherapy, 1995, 41, 247-263; K. Licha et al., New contrast agent for optical imaging: acid-cleavable conjugates of cyanine dyes with biomolecules, In Biomedical Imaging: Reporters, Dyes, and Instrumentation, D. J. Bomhop, C. Contag, and E. M. Sevick-Muraca (Eds.), Proceedings of SPIE, 1999, 3600, 29-35, each of which are expressly incorporated by reference herein in their entirety. Therefore, the inventive receptor-targeted phototherapeutic agents are expected to be effective in the treatment of various lesions.

The estrogen receptor is an example of a steroid receptor to which steroid receptor binding molecules would bind. The following compounds are known to bind to the estrogen receptor: estratriol, 17β-aminoestrogen (AE) derivatives such as prolame and butolame; drugs such as tamoxifen, ICI-164384, raloxifene, genistein; 17β-estradiol; glucocorticoids, progesterone, estrogens, retinoids, fatty acid derivatives, phytoestrogens, etc. In addition, commercially available kits can identify compounds specific for binding to the estrogen receptor (e.g., Estrogen Receptor-alpha Competitor Assay Kit, Red; Estrogen Receptor-beta Competitor Assay Kit, Red (Invitrogen Corp., Carlsbad Calif.).

The glucose receptor is an example of a carbohydrate receptor to which carbohydrate receptor binding molecules would bind. The glucose conjugate N-palmitoyl glucosamine [NPG] is known to bind the glucose receptor (Dufes et al., Pharm. Res. 17:1250, 2000). The glycoprotein hormone receptor is another example of a carbohydrate receptor to which carbohydrate receptor binding molecules would bind. Follicle stimulating hormone (FSH) is known to bind the glycoprotein hormone receptor (Tilly et al., Endocrinology 131: 799, 1992). Other compounds known to bind the carbohydrate receptor, and hence examples of carbohydrate receptor binding molecules, are polysialic acid, bacterial adhesins (specialized surface proteins that mediate binding of many pathogenic bacteria, such as enterohemorrhagic E. coil (EHEC) and Shigella dysenteriae, to host cells, which allow these bacteria to colonize host cell surfaces), soluble carbohydrate receptor analogs, artificial glycopolymers and other multivalent glycoconjugates such as an acrylamide copolymer carrying -L-fucopyranoside and 3-sulfo-D-galactopyranoside in clusters, isomeric carbohydrates, synthetic derivatives, neoglycoproteins, neoglycolipids, glycosidases, and glycosyltransferases. Carbohydrate binding proteins can be screened with phage display libraries as known to a person of ordinary skill in the art.

Somatostatin receptor binding molecules include somatostatin and somatostatin receptor analogs, octreotide, glycosylated somatostatin-14 (somatostatin-dextran⁷⁰), seglitide, peptides P587 and P829 as described in Vallabhajosula et al., J. Nuclear Med., 37:1016, 1996.

Cholecystokinin receptor binding molecules include the endogenous peptides cholecystekinin (CCK)-4, CCK-8, CCK-33, and gastrin; antagonists devazepide and lorglumide; agonists BC264 [Tyr(SO₃H)-gNle-mGly-Trp-(NMe)Nle-Asp-Phe-NH₃] and desulfated CCK-8; Kinevac (synthetic cholecystekinin, sincalide); and CCK analogues modified at the sulfated tyrosyl at position 27.

Neurotensin receptor binding molecules include neurotensin, neuromedin N, JMV449 (H-Lysψ(CH₂NF)-Lys-Pro-Tyr-Ile-Leu), the non-peptide antagonist SR142948A (2-([5-(2,6-dimethoxyphenyl)-1-(4-(N-[3-dimethylaminopropyl]-N-methylcarbamoyl)-2-isopropylphenyl)-1H-pyrazole-3-carbonyl)amino)adamantine-2 -carboxylic acid hydrochloride), and levocobastine. Commercially available neurotensin receptor binding kits can evaluate potential neurotensin receptor binding molecules (e.g., DELFIA Neurotensin Receptor Binding Kit, PerkinElmer (Boston Mass.)).

Bombesin receptor binding molecules include the endogenous ligands gastrin-releasing peptide (GRP), neuromedin B (NMB), and GRP-18-27, and antagonists including JMV-1458 (glycine-extended bombesin (paraphydroxy-phenyl-propionyl-Gln-Trp-Ala-Val-Gly-His-Leu-Met-Gly-OH)), JMV-641, JMV-1799, and JMV-1802, PD165929, 1-naphthoyl-[DAla²⁴, DPro²⁶, ψ26-27]GRP-20-27, kuwanon H, and kuwanon G. Commercially available bombesin receptor binding kits can evaluate potential bombesin receptor binding molecules (e.g., DELFIA Bombesin Receptor Binding Kit, PerkinElmer (Boston Mass.)).

ST receptor binding molecules include native ST peptide, and SEQ ID NO:2, SEQ ID NO:3, SEQ ID NOS:5-54 and fragments and derivatives thereof from U.S. Pat. No. 5,518,888.

In one example, a targeting ligand may contain all or part of a steroid hormone or a steroid receptor binding compound, and therefore target steroid hormone sensitive receptors. In this example, the compound is administered, targets and preferably accumulates in the desired site such as breast and/or prostate lesion, is photoactivated, and forms free radicals at this site thereby effecting cell injury, damage, or death at the desired target site. Similar target binding molecules and uses will be recognized by one skilled in the art. For example, the targeting group may be a compound that targets and binds to a somatostatin, bombesin, CCK, and/or neurotensin receptor binding molecule, or may be a carcinogenic embryonic antigen-binding compound that binds to a carcinogenic embryonic antigen. These are then photoactivated for radical formation at, for example, lung cancer cells with CCK receptor binding molecules, colorectal cancer cells with ST receptor and carcinoembryonic antigen (CEA) binding molecules, melanoma cells with dihyroxyindolecarboxylic acid, vascular sites of atherosclerotic plaque with integrin receptor binding molecules, brain lesions with amyloid plaque binding molecules, and the like.

Successful specific targeting of photoactive compounds to tumors using antibodies and peptides for diagnostic imaging of tumors has been described in Achilefu et al., Novel receptor-targeted fluorescent contrast agents for in vivo imaging of tumors, Investigative Radiology, 2000, 35, pp. 479-485; Ballou et al., Tumor labeling in vivo using cyanine conjugated monoclonal antibodies, Cancer Immunology and Immunotherapy, 1995, 41, pp. 257-263; and Licha et al., New contrast agent for optical imaging: acid cleavable conjugates of cyanine dyes with biomolecules, in Biomedical Imaging: Reporters, Dyes and Instrumentation, Proceedings of SPIE, 1999, 3600, pp. 29-35. As such, it is widely accepted that targeted photochemicals are effective in targeting, detecting and treating a wide range of physiological/biological sites.

The optical agents of this example may contain additional functionalities that can be used to attach various types of biomolecules, synthetic polymers, and organized aggregates for selective delivery to various organs or tissues of interest. Examples of synthetic polymers include polyaminoacids, polyols, polyamines, polyacids, oligonucleotides, aborols, dendrimers, and aptamers. The invention includes, but is not limited to, phototherapeutic agents comprising a photosensitizer—biomolecule conjugate which provides advantages over nonspecific phototherapeutic agents or the conjugation of photosensitizers to very large biomolecules. These conjugates provide enhanced localization in, and rapid visualization of, tumors which is beneficial for both diagnosis and therapy. The agents are rapidly cleared from blood and non-target tissues so there is less concern for accumulation and for toxicity. A variety of high purity compounds may be easily synthesized for combinatorial screening of new targets, e.g., to identify receptors or targeting agents, and for the ability to affect the pharmacokinetics of the conjugates by minor structural changes.

In some embodiments, a liposome or micelle may be utilized as a carrier or vehicle for the composition. For example, in some embodiments, a phototherapeutic agent comprises a fused ring azo or diaza photosensitizer that may be a part of the lipophilic bilayers or micelle, and the targeting ligand, if present, may be on the external surface of the liposome or micelle. As another example, a targeting ligand may be externally attached to the liposome or micelle after formulation for targeting the liposome or micelle (which contains the fused ring azo or diaza phototherapeutic agent/photosensitizer) to the desired tissue, organ, or other site in the body.

In a specific aspect, the invention provides a fused ring azo or diaza compound for use in a phototherapy procedure, the compound being of the formula (FX29):

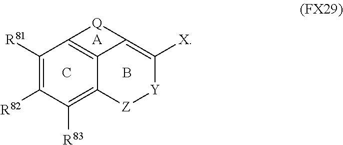

With regard to this aspect, Q is —C(R⁸⁸R⁸⁹)N═N—, —C(R⁸⁸)═NN(R⁸⁹)—, or —N═N—. In an embodiment, each of R⁸⁸ and R⁸⁹ is independently hydrogen, C₁-C₁₀ alkyl, C₅-C₁₀ aryl, C₁-C₁₀ alkoxyalkyl, C₁-C₁₀ polyhydroxyalkyl, —(CH₂)ₙCO₂R⁹⁰, or —(CH₂)ₚNR⁹¹R⁹². Further, R⁹⁹ can be either hydrogen or C₁-C₁₀ alkyl, and each of R⁹¹ and R⁹² can independently be hydrogen, C₁-C₁₀ alkyl, C₅-C₁₀ aryl, C₁-C₁₀ polyhydroxyalkyl, —(CH₂)_{q}CO₂R⁹³, or —COR⁹⁴. In the case that one or both of R⁹¹ and R⁹² is —(CH₂)_{q}CO₂R⁹³ and/or —COR⁹⁴, R⁹³ can be either hydrogen or C₁-C₁₀ alkyl, and R⁹⁴ can be hydrogen, C₁-C₁₀ alkyl, C₁-C₁₀, alkoxyalkyl, or C₁-C₁₀ polyhydroxyalkyl.

Still referring to compounds of formula (FX29) above, in an embodiment, each of R⁸¹, R⁸², and R⁸³ is independently hydrogen, C₁-C₁₀ alkyl, C₅-C₁₀ aryl, hydroxyl, —SO₃H, C₁-C₁₀ alkoxy, C₁-C₁₀ polyhydroxyalkyl, C₁-C₁₀ polyalkoxyalkyl, —(CH₂)ᵣCO₂R⁹⁵, —(CH₂)ₛNR⁹⁶R⁹⁷, or —(CH₂)ₜCONR⁹⁸R⁹⁹. Further, X is either hydrogen or halogen (e.g., fluorine, chlorine, bromine, iodine, or astatine). In addition, Y can be —CR⁸⁴R⁸⁵—, —O—, —NR⁸⁴—, —S—, or —C(O)—. Similarly, Z can be —CR⁸⁶R⁸⁷—, —O—, —NR⁸⁶—, —S—, or —C(O)—.

There are various “R” groups mentioned as possible components of R⁸¹, R⁸², R⁸³, Y, a Z. In embodiments which have such “R” groups, R⁹⁵ can be either hydrogen or C₁-C₁₀ alkyl. Further, each of R⁹⁶, R⁹⁷, R⁹⁸, R⁹⁹, R⁸⁴, and R⁸⁶ can independently be hydrogen, C₁-C₁₀ alkyl, C₁-C₁₀ alkoxyalkyl, or C₁-C₁₀ polyhydroxyalkyl. In addition, each of R⁸⁴, R⁸⁵, R⁸⁶, and R⁸⁷ can independently be hydrogen, C₁-C₁₀ alkyl, C₅-C₁₀ aryl, C₁-C₁₀ alkoxyalkyl, C₁-C₁₀ polyhydroxyalkyl, —(CH₂)ᵤCO₂R¹⁰⁰, —(CH₂)ᵥNR¹⁰¹R¹⁰², or —(CH₂)_{w}CONR¹⁰³R¹⁰⁴. R¹⁰⁰ can be hydrogen or C₁-C₁₀ alkyl, and each of R¹⁰¹, R¹⁰², R¹⁰³, and R¹⁰⁴ can independently be hydrogen, C₁-C₁₀ alkyl, C₁-C₁₀ alkoxyalkyl, or C₁-C₁₀ polyhydroxyalkyl.

There are also a number of variables mentioned in regard to compounds of formula (FX29). These variables may be any appropriate integers. In the context of the description of formula (FX29), for instance, each of “n”, “p”, “q”, “r”, “s”, “t”, “u”, “v”, and “w” may independently be 0, 1, 2, 3, 4, 5, 6, 7, 8, 9, or 10. In some embodiments, one or more (e.g., each) of “n”, “p”, “q”, “r”, “s”, “t”, “u”, “v”, and “w” may independently be 0, 1, 2, 3, 4, 5, or 6.

Compounds of formula (FX29) above also include the possibility of various substituents forming additional ring structures. For instance, R⁸¹ and R⁸² may be tethered together to form a five- or six-membered ring. As another example, R⁸² and R⁸³ may be tethered together to form a five- or six-membered ring. As still another example, in the case that both of Y and Z include R groups, the R groups of Y and Z may be tethered together to form a five- or six-membered ring.

Various refinements exist for compounds of the first aspect. These refinements can occur individually or in any appropriate combination. To promote conciseness, each and every possible combination of refinements may not be expressly described herein. However, this disclosure is to be interpreted as if each and every possible combination of refinements is indeed expressly described.

For example, in some embodiments, X may be hydrogen, while X may be halogen in other embodiments. In one particular example, X may be bromine.

As mentioned above, Q may be —C(R⁸⁸R⁸⁹)N═N— or —C(R⁸⁸)═NN(R⁸⁹)— in some embodiments. In some of such embodiments, each of R⁸⁸ and R⁸⁹ may independently be hydrogen or C₁-C₁₀ alkyl rather than substituents of the broader list mentioned above.

Y of formula (FX29) can be —CR⁸⁴R⁸⁵—, —O—, —NR⁸⁴—, —S—, or —C(O)—. As such, in some embodiments, Y is —CR⁸⁴R⁸⁵—. In other embodiments, Y is —C(O)—. In still other embodiments, Y is —O—. In yet other embodiments, Y is —NR⁸⁴—. In still other embodiments, Y is —S—.

Similarly, Z of formula (FX29) can be —CR⁸⁶R⁸⁷—, —O—, —NR⁸⁶—, —S—, or —C(O)—. As such, in some embodiments, Z is —CR⁸⁶R⁸⁷—. In other embodiments, Z is —O—. In still other embodiments, Z is —NR⁸⁸—. In yet other embodiments, Z is —S—. In still other embodiments, Z is —C(O)—.

Various possible refinements exist for each of R⁸¹, R⁸², and R⁸³ of formula (FX29) as well. For instance, in some embodiments, each of R⁸¹, R⁸², and R⁸³ may independently be hydrogen, hydroxyl, C₁-C₁₀ alkoxy, —SO₃H, or —(CH₂)ᵣCO₂R⁹⁵. In other embodiments, each of R⁸¹, R⁸², and R⁸³ may independently be hydrogen, hydroxyl, or C₁-C₁₀ alkoxy. In some embodiments, R⁹⁵ is hydrogen or C₁-C₁₀ alkyl. In an embodiment, each of R⁸⁴-R⁸⁷ and R⁹⁵-R¹⁰⁴ is independently H or C₁-C₁₀ alkyl.

In one exemplary family of embodiments, Q is —C(R⁸⁸R⁸⁹)N═N— or —C(R⁸⁸)═NN(R⁸⁵)—, and each of R⁸⁸ and R⁸⁹ is hydrogen. Further, each of R⁸¹, R⁸², and R⁸³ is independently hydrogen or C₁-C₁₀ alkoxy (e.g., C₁-C₃ alkoxy, such as methoxy). X is hydrogen or halogen (e.g., bromine), Y is —C(O)—, and Z is —O—.

Some compounds of formula (FX29) tend to operate through the Type 1 mechanism wherein the —N═N— bond undergoes nitrogen extrusion upon photoactivation, thereby producing at least one free radical. Some compounds of formula (FX29) tend to operate through the Type 1 mechanism wherein the C—X bond is cleaved upon photoactivation, thereby producing at least one free radical. Some compounds of formula (FX29) tend to operate through the Type 1 mechanism wherein a C—N bond is cleaved upon photoactivation, thereby producing at least one free radical. Without being bound by a particular theory, photoexcitation of the aromatic chromophore is believed to effect rapid intramolecular energy transfer to the intra-ring azo or diaza group of ring A, resulting in N—C, N—N or C—X bond rupture, optionally with concomitant extrusion of molecular nitrogen and formation of radicals. These and other aspects of the Type 1 mechanism are provided in U.S. Pat. Nos. 6,485,704 and 6,747,151, each of which is incorporated herein by reference to the extent that such references are not inconsistent with the teachings herein. Bioconjugates, incorporating compounds of formula (FX29) and a biomolecule, and methods of using the present compounds and bioconjugates are also provided. Such bioconjugates can be prepared by methods well known in the art, such as those methods provided herein and in U.S. Pat. Nos. 6,485,704 and 6,747,151.

In a specific embodiment of this aspect, a fused ring azo compound of the invention has Formula (FX32).

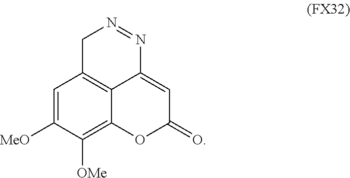

In a specific embodiment of this aspect, a fused ring diaza compound of the invention has Formula (FX33).

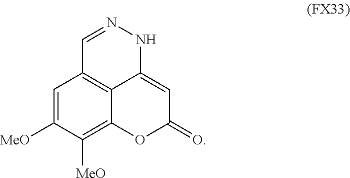

In another specific aspect, the invention provides bioconjugates of compounds having formula (FX29). In particular, a bioconjugate of this aspect includes a compound of any one of formula (FX29)-(FX33) and a biomolecule linked (either directly through an appropriate bond or indirectly via an appropriate linking group) to the compound. The biomolecule is typically an appropriate moiety useful in directing/targeting the compound to a particular target (e.g., cell, tissue, receptor, etc.). For instance, in some embodiments, the biomolecule may be an antibody, a peptide, a peptidomimetic, a carbohydrate, a glycomimetic, a drug, a hormone, or a nucleic acid. In some embodiments, the biomolecule may be a somatostatin receptor binding molecule, a heat-sensitive bacterioenterotoxin (ST) receptor binding molecule, a neurotensin receptor binding molecule, a bombesin receptor binding molecule, a cholecystekinen (CCK) receptor binding molecule, a steroid receptor binding molecule, or a carbohydrate receptor binding molecule.

A related third aspect of the invention is directed to methods for making the above-mentioned bioconjugates. In such methods, one or more appropriate steps and conditions are taken to link (either directly through an appropriate bond or indirectly via an appropriate linking group) an above-mentioned biomolecule to a compound of formula (FX29)-(FX33). Such a method may include displacement of one or more atoms/molecules (e.g., hydrogen, an alkyl group, or an appropriate R group) from the compound such that the biomolecule can be appropriately linked to the compound to form the resulting bioconjugate. For example, in some embodiments, the biomolecule may be linked to the fuse ring core of the compound via, —(CH₂)ₙ—, —(HCCH)ₙ—, —O—, —S—, —SO—, —SO₂—, —SO₃—, —OSO₂—, —NR¹⁴—, —CO—, —COO—, —OCO—, —OCOO—, —CONR¹⁵—, —NR¹⁶CO—, —OCONR¹⁷—, —NR¹⁸COO—, —NR¹⁹CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ—, —OCO(CH₂)ₙ—, —OCOO(CH₂)ₙ—, —CONR²⁴(CH₂)ₙ—, —CONR²⁵(CH₂)ₙ—, —NR²⁶CO(CH₂)ₙ—, —OCONR²⁷(CH₂)ₙ—, —NR²⁸COO(CH₂)ₙ—, —NR²⁹CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—.

As an example of a possible refinement of the linker portion of bioconjugates of the invention, the biomolecule may be linked to the compound via —NR¹⁶CO(CH₂)_{q}—, —(CH₂)ₚCONR¹⁵—; —OCONR¹⁷—, —NR¹⁸COO—, —NR¹⁹CONR²⁰—, or —NR¹⁹CO(CH₂)ᵣCONR²⁰— in some embodiments, wherein R¹⁵-R²⁰ are as described in connection with formula (FX1). As another possible refinement, at least one (e.g., each) of “p”, “q”, and “r” may independently be 0, 1, 2, 3, 4, 5, or 6.

One specific example of a bioconjugate of a compound of formula (FX29) is shown below in Formula (FX34) and (FX35):

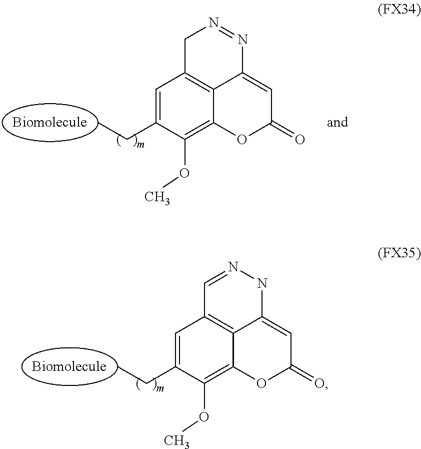

wherein m is an integer ranging from 1 to 100, optionally 1 to 10. In an embodiment, the “Biomolecule” component in formula (FX34) and (FX35) is Bm as described in connection with formula (FX1), and optionally is a polypeptide.

Another specific example of a bioconjugate of a compound of formula (FX29) is shown below in Formula (FX36) and (FX37):

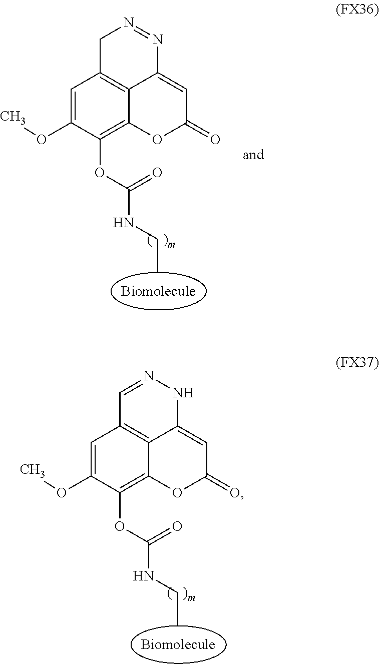

wherein m is an integer ranging from 1 to 100, optionally 1 to 10. In an embodiment, the “Biomolecule” component in formula (FX36) and (FX37) is Bm as described in connection with formula (FX1), and optionally is a polypeptide

Yet another specific example of a bioconjugate of a compound of formula (FX29) is shown below in Formula (FX38) and (FX39):

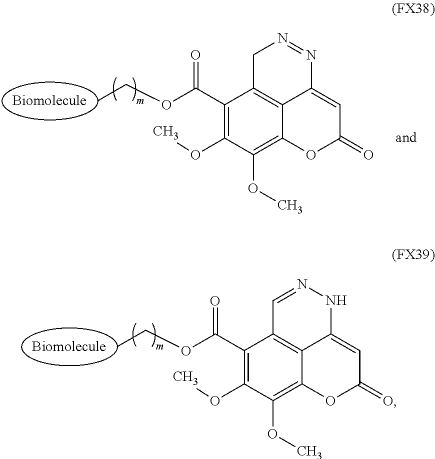

wherein m is an integer ranging from 1 to 100, optionally 1 to 10. In an embodiment, the “Biomolecule” component in formula (FX38) and (FX39) is Bm as described in connection with formula (FX1), and optionally is a polypeptide

### 1b. Synthesis of Phototherapeutic Agents

Methods for synthesizing and derivatizing fused ring azo and diaza compounds are known in the art. For example, properties of fused ring azo and diaza compounds and methods of synthesizing and derivatizing fused ring azo and diaza compounds are described in Carpino L. Journal of Organic Chemistry, 1994, 59, 3564-3571; and Tsukamoto, M. et al. WO 2000013508 (UX 6573218), which are hereby incorporated by reference in their entireties.

### 1.b.(i) Preparation of the Compound of Formula (FX33)

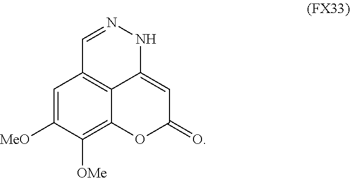

Hydrazine hydrate (60 mg; 1 mmol) in THF (1 mL) was added to a solution of 5-bromo-methyl-4,7,8-trimethoxycoumarin (0.4 g; 1 mmol) in THF (4 mL) and the entire mixture was heated under reflux for two hours. TLC indicated complete consumption of the starting material. The solvent was evaporated in vacuo and the residue purified by automated flash chromatography over silica gel using a methanol/chloroform gradient (0 to 5% methanol over one hour). The desired fractions were pooled and evaporated in vacua to give the desired material, which formed as a electromagnetic radiation brown solid.

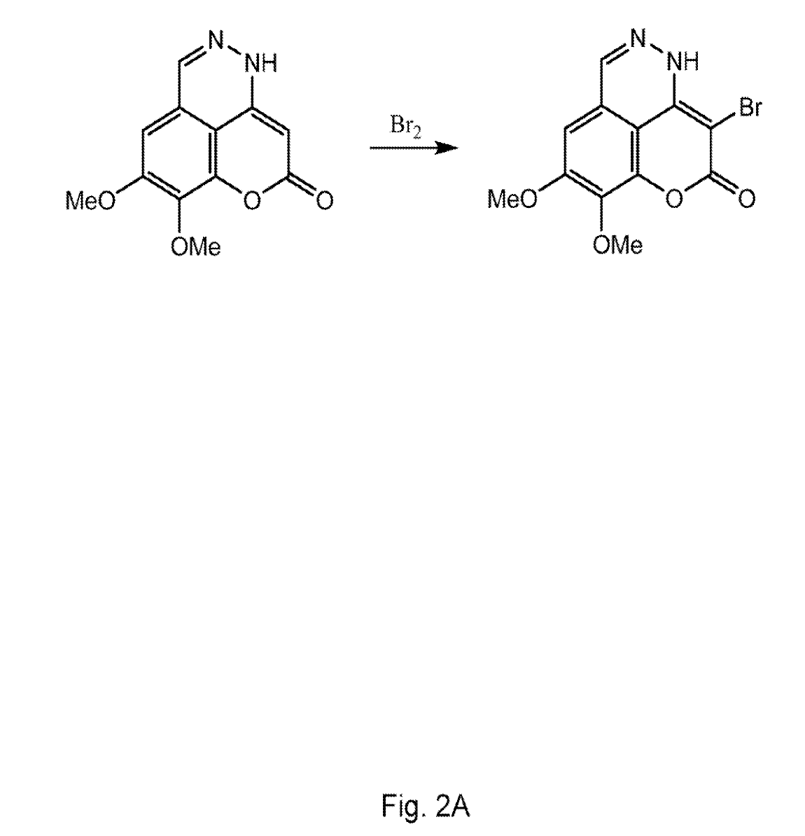

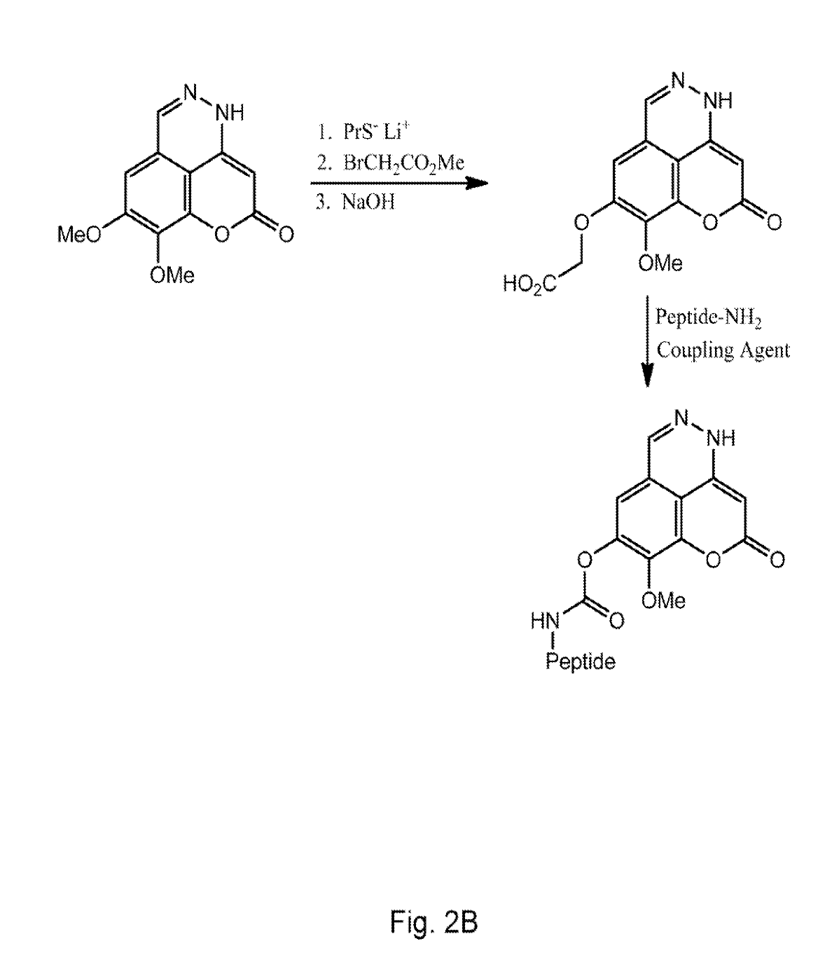

FIGS. 2A and 2B provide examples of synthetic schemes for derivatizing fused ring diaza photosensitizers of the invention. FIG. 2A provides a scheme for the addition of Br to unsaturated ring B of the fused ring diaza compound having formula (FX33), thereby resulting in synthesis of a compound having formula (FX40).

### 1.b(ii) Preparation of the Compound of Formula (FX40)

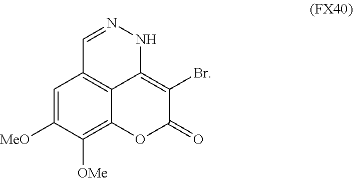

A mixture of a compound having formula (FX33) (246 mg, 1 mmol) is dissolved in methylene chloride (10-20 mL) and cooled to 0-5° C. Thereafter, bromine (176 mg, 1.1 mmol) is added and the entire mixture is stirred at ambient temperature until the bromine solution is decolorized. The reaction mixture is then treated with saturated sodium bicarbonate solution (20 mL) and the organic layer is separated, washed with additional saturated bicarbonate solution and water, dried over anhydrous sodium sulfate, filtered, and the filtrate evaporated in vacuo. The crude product having formula (FX40) is then purified by flash chromatography.

### 1.b(iii) General Synthesis Fused Ring Azo or Diaza Compound Bioconjugates

FIG. 2B provides a scheme showing an exemplary method for making a fused ring diaza bioconjugate. As shown in FIG. 2B, a fused ring diaza compound having formula (FX33) is coupled to a peptide targeting ligand. As will be understood by those having skill in the art, similar synthetic approaches may be used to generate bioconjugates of the azo fused ring compounds of the invention.

### EXAMPLE 1.b(iv)

### Phototherapeutic Methods and Cell Viability Measurements

A general procedure is carried out for measuring cell viability upon exposure of tumor cells to the fused ring diaza compound of Formula (FX33) and electromagnetic radiation. The compound of Formula (FX33) has an absorption maxima (λₘₐₓ) of 334 nm. The cell viability measurements are carried out using human myeloid leukemia U937 cell line by the standard WST-1 assay. In this procedure, U397 Leukemia cells (0.5×10⁶) are plated in standard T-25 cell culture flasks, and are exposed to four controls and a series of test conditions corresponding to a range of fused ring diaza compound concentrations, summarized in Table 1.

TABLE 1
Control and Test Conditions for Cell Viability Measurements

| | |
|---|---|
| Control 1 | no electromagnetic radiation, no photosensitizer |
| Control 2 | electromagnetic radiation, no photosensitizer |
| Control 3 | no electromagnetic radiation, photosensitizer |
| Control 4 | electromagnetic radiation, dimethylsulfoxide |
| | (DMSO), no photosensitizer |
| Test Condition | electromagnetic radiation, fused ring diaza |
| | compound photosensitizer |

The compound of formula (FX33) is dissolved in 20 to 30% DMSO at an initial concentration of 2-4 mM and serially diluted to the final desired value such that the amount of DMSO exposed to the cells is below 0.5%. The cells are incubated at 37° C. with various concentrations of the fused ring diaza compound photosensitizer for about 30 minutes prior to exposure to electromagnetic radiation. The cells are irradiated at 5, 10 and 20 and 30 minute durations using a 200-W UV lamp that radiates electromagnetic radiation with maximum output in the range of 325-425 nm with constant cooling such that the temperature at the surface of the microliter plates does not exceed 37° C. It should be noted that in the present study, the electromagnetic radiation source is not optimized with respect to power and wavelength. The viability of cells is assessed after 24 hours following exposure to electromagnetic radiation. The electromagnetic radiation source is a B-100SP High Intensity Lamp from UVP. Once exposure is complete, cells are processed to determine percent viability using Hank's Balanced Salt Solution (HBSS), Trypan blue stain, and a hemacytometer to count live and dead cells. The number of viable cells is determined and percent viability is determined using:

$$Percent Viability = \frac{No . of Viable Cells Counted \times 100}{Total No . of Cells Counted}$$

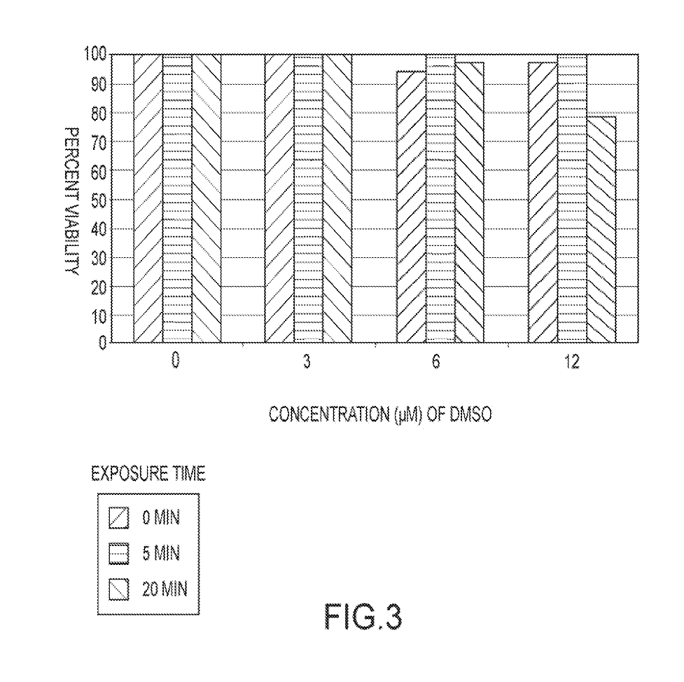

FIG. 3 provides cell viability results for control conditions (Control 4, no photosensitzer, DMSO, electromagnetic radiation) wherein cells are exposed to electromagnetic radiation and dimethyl sulfoxide. As shown in FIG. 3, DMSO toxicity is observed only at high concentrations of DMSO. Cells are incubated without DMSO (0 μM) and with DMSO at concentrations of 3 μM, 6 μM, and 12 μM, and are exposed to electromagnetic radiation from a B-100SP High Intensity Lamp for 0, 5, and 20 minutes. The results indicate that 100% of the cells are viable for conditions of 0 μM DMSO and 3 μM DMSO with no exposure to electromagnetic radiation, electromagnetic radiation exposure for 5 minutes, and electromagnetic radiation exposure for 20 minutes. At least about 95% of the cells are viable for conditions of 6 μM DMSO with no electromagnetic radiation exposure, electromagnetic radiation exposure for 5 minutes, and electromagnetic radiation exposure for 20 minutes. With 12 μM DMSO with no electromagnetic radiation exposure and with electromagnetic radiation exposure for 5 minutes, at least 95% of cells were viable; and with electromagnetic radiation exposure for 20 minutes, about 78% of cells are viable.

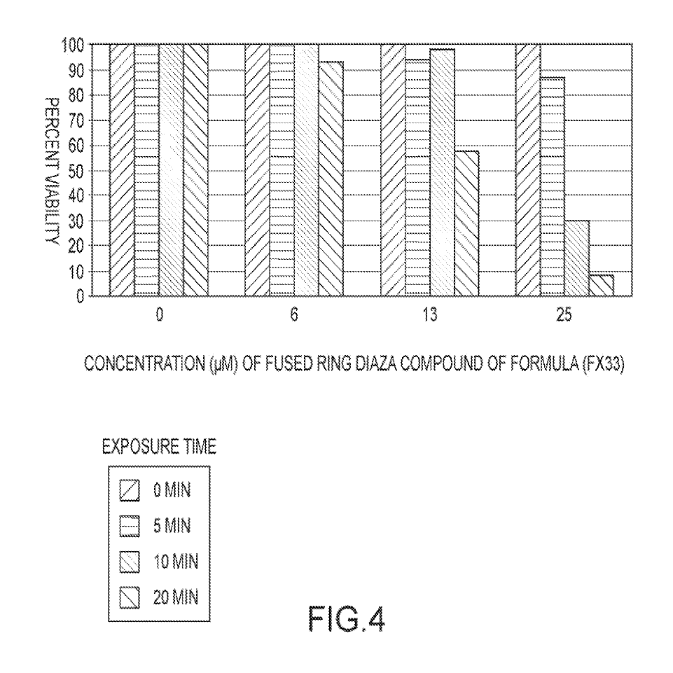

FIG. 4 provides cell viability results for test conditions wherein cells are exposed to electromagnetic radiation and the compound of formula (FX33) for concentrations of 6 μM, 13 μM and 25 μM. The cell viability experiments with the compound of formula (FX33) exhibit concentration-dependent and electromagnetic radiation exposure time-dependent decreases in cell viability. As shown in FIG. 4, the viability of cells in contact with a fused ring diaza photosensitizer decreases dramatically upon exposure to electromagnetic radiation over the range of 5 minutes to 20 minutes. Cells are incubated in the absence of the compound of formula (FX33) (0 μM) and with the compound of formula (FX33) at concentrations of 6 μM, 13 μM and 25 μM. The cells are exposed to electromagnetic radiation from a B-100SP High Intensity Lamp for 0, 5, 10, and 20 minutes. The results indicate that in the absence of the compound of formula (FX33), cells at all electromagnetic radiation exposure times have 100% viability. in the presence of 6 μM of the compound of formula (FX33), cells have 100% viability with electromagnetic radiation exposure for 5 and 10 minutes, and about 92% viability with electromagnetic radiation exposure for 20 minutes. In the presence of 13 μM of the diaza compound of formula (FX33), cells have about 94% viability with electromagnetic radiation exposure for 5 minutes, about 95% viability with electromagnetic radiation exposure for 10 minutes, and about 58% viability with electromagnetic radiation exposure for 20 minutes. In the presence of 25 μM of the compound of formula (FX33), cells have about 86% viability with electromagnetic radiation exposure for 5 minutes, about 30% viability with electromagnetic radiation exposure for 10 minutes, and about 8% viability with electromagnetic radiation exposure for 20 minutes. A VC_{50/20}value of 5.25 μM is determined for the compound of formula (FX33), wherein VC_{50/20} is defined as the concentration at which 50% decrease in cell viability is observed when the cells are exposed to electromagnetic radiation and the fused ring diaza photosensitizer for 20 minutes.

### EXAMPLE 2

### Phototherapy Methods

Phototherapy, such as photodynamic therapy (PDT), typically employs a combination of a photosensitizer (PS) and visible or near infrared electromagnetic radiation to generate reactive species that kill or otherwise degrade target cells, such as tumors or other lesions. The invention provides phototherapeutic agents useful for phototherapy.

The invention includes phototherapy methods wherein a phototherapeutic agent comprising a compound of any one of the formulas (FX1)-(FX40) is administered to a patient, for example, wherein a therapeutically effective amount of such a component is administered to a patient in need of treatment. In some embodiments, compounds of the invention provide an optical agent capable of selective targeting and delivery to a target tissue such as a tumor, site of inflammation or other lesion. Upon administration, the phototherapeutic agent is optionally allowed to accumulate in a target region of interest (e.g., target tissue, tumor, or organ). To induce selective tissue damage, the phototherapeutic agent is activated by exposure to electromagnetic radiation, preferably for some methods at the site of the target tissue. In an embodiment, the phototherapeutic agent is activated after a therapeutically or diagnostically effective concentration of the phototherapeutic agent has accumulated in a target tissue. An effective concentration of a compound of the invention depends on the nature of the formulation, method of delivery, target tissue, activation method and toxicity to the surrounding normal non-target tissue. Exposure to electromagnetic radiation and activation of the phototherapeutic agent may occur during or after administration of the phototherapeutic agent and accumulation at the target tissue.

For photoactivation, the target region is illuminated with electromagnetic radiation having wavelengths in the range of about 350 nm to about 1300 nm, preferably for some applications in the range of about 400 nm to about 900 nm. In an embodiment, the target region is illuminated with electromagnetic radiation having wavelengths in the range of about 300 nm to about 900 nm. In some embodiments, the wavelength of the electromagnetic radiation corresponds to a peak in the absorption spectrum of the phototherapeutic agent, for example is within 20 nanometers of a peak in the absorption spectrum of the phototherapeutic agent in the visible or NIR regions. In some phototherapeutic procedures the target site is exposed to electromagnetic radiation having fluence, dosage and/or power sufficient to activate the phototherapeutic agent so as to induce cell death, for example via necrosis or apoptosis processes. In some embodiments, electromagnetic radiation having low energy (e.g., less than 200 mW/cm² or optionally less than 100 mW/cm²), power or fluence, but sufficient dosage, is provided to activate the phototherapeutic agent without undesirable thermal effects. If the region of interest is, for example, a lesion or tumor on the skin surface, the region can be directly illuminated. In some methods, endoscopic and/or endoluminal catheters can be employed to deliver electromagnetic radiation to the subject to provide a photodiagnostic and/or phototherapeutic effect.

Appropriate power, dosage and intensity of the electromagnetic radiation depends on the size, depth, and the pathology of the lesion, as is known to one skilled in the art. In an embodiment, the fluence of the electromagnetic radiation is preferably, but not always, kept below 200 mW/cm² , optionally below 100 mW/cm², to minimize undesirable thermal effects. The intensity, power, and duration of the illumination, and the wavelength of the electromagnetic radiation may vary widely depending on the body location, the lesion site, the effect to be achieved, etc. In an embodiment, the power of the applied electromagnetic radiation is preferably is selected over the range of 1-500 mW/cm² and optionally for some applications is selected over the range of 1-200 mW/cm² and optionally for some applications selected over the range of 1-100 mW/cm². In an embodiment, the duration of the exposure to applied electromagnetic radiation is selected over the range of 1 second to 60 minutes, and optionally for some applications is selected over the range of 1 second to 30 minutes, and optionally for some applications is selected over the range of 1 second to 10 minutes, and optionally for some applications is selected over the range of 1 second to 1 minute.

In an embodiment, the invention provides a method of using a phototherapeutic agent, the method comprising: (1) administering a therapeutically effective amount of a phototherapeutic agent to a subject, the phototherapeutic agent comprising a compound being of the formula (FX1):

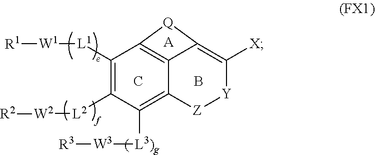

or a pharmaceutically acceptable salt or ester thereof, wherein:

Y is —CU^{a}U^{b}—, —NU^{a}—, —O—, —S—, or —C(O)—;

Z is —CU^{c}U^{d}—, —NU_{c}—, —O—, S—, or —C(O)—;

wherein each U^{a} is independently -(L⁴)ₕ-W⁴—R⁴;

wherein each U^{b} is independently -(L⁵)ᵢ-W⁵—R⁵;

wherein each U^{c} is independently -(L⁶)ⱼ-W⁶—R⁶;

wherein each U^{d} is independently -(L⁷)ₖ-W⁷—R⁷;

X is hydrogen, F, Cl, Br, I, or At;

Q is —C(R⁸R⁹)N═N—, —C(R⁸)═NN(R⁹)—, or —N═N—,

each of L¹-L⁷, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;

each of W¹-W⁷ is independently a single bond, —(CH₂)ₙ—, —(HCCH)ₙ—, —O—, —S—, —SO—, —SO₂—, —SO₃—, —OSC₂—, —NR¹⁴—, —CO—, —COO—, —OCO—, —OCOO—, —CONR¹⁵—, —NR¹⁶CO—, —OCONR¹⁷—, —NR¹⁸COO—, —NR¹⁹CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ—, —OCO(CH₂)ₙ—, —OCOO(CH₂)ₙ—, —CONR²⁴(CH₂)ₙ—, —CONR²⁵(CH₂)ₙ—, —NR²⁶CO(CH₂)ₙ—, —OCONR²⁷(CH₂)ₙ—, —NR²⁶COO(CH₂)ₙ—, —NR²⁹CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—;

each of R¹-R⁹ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₂₀ alkoxy, C₁-C₂₀ alkoxycarbonyl, C₁-C₂₀ alkoxyalkyl, C₁-C₁₀ polyhydroxyalkyl, C₁-C₁₀ polyalkoxyalkyl, halo, halomethyl, dihalomethyl, trihalomethyl, —CO₂R⁴⁰, —SOR⁴¹, —OSR⁴², —SO₂OR⁴³, —CH₂(CH₂OCH₂)_{b}CH₂OH, —PO₃R⁴⁴R⁴⁵, —OR⁴⁶, —SR⁴⁷, —NR⁴⁸R⁴⁹, —NR⁵⁶COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —SO₃R⁶², —CH(R⁶³)CO₂H, —CH(R⁶⁴)NH_{2,} FL or Bm; or wherein R¹, R², W¹, W², and L¹ and L², if present, together with the atoms to which they are attached combine to form one or more carbocyclic or heterocyclic 5, 6, or 7 membered rings; or wherein R², R³, W², W³, and L² and L³, if present, together with the atoms to which they are attached combine to form one or more carbocyclic or heterocyclic 5, 6, or 7 membered rings; or wherein U^{a} and U^{c} together with the atoms to which they are attached combine to form one or more carbocyclic or heterocyclic 5, 6, or 7 membered rings; or wherein R³, W³, and L³, if present, and U^{c} together with the atoms to which they are attached combine to form one or more carbocyclic or heterocyclic 5, 6, or 7 membered rings;

each of a and b is independently an integer selected from the range of 1 to 100;

each of n is independently an integer selected from the range of 1 to 10;

each of e, f, g, h, i, j, and k is independently 0 or 1;

each of R¹⁴-R³⁶ is independently hydrogen, C₁-C₁₀ alkyl, C₁-C₁₀ alkoxy, C₅-C₁₀ aryl, C₁-C₁₀ alkoxyalkyl, C₁-C₁₀ polyhydroxyalkyl, —(CH₂)ₙCO₂R⁶⁵, or —COR⁶⁶;

each of R⁴⁰-R⁶² and R⁶⁵-R⁶⁶ is independently hydrogen, C₁-C₁₀ alkyl, C₁-C₁₀ alkoxy, C₁-C₁₀ alkoxyalkyl, or C₁-C₁₀ polyhydroxyalkyl;

each of R⁶³ and R⁶⁴ is independently a side chain residue of a natural α-amino acid;

each of FL is independently a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine; and

each Bm is independently a group corresponding to an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steriod, a hormone, an aptamer, a receptor, a metal chelating agent, a mono- or polynucleotide comprising 1 to 50 nucleic acid units, or a polypeptide comprising 2 to 30 amino acid units; and (ii) exposing the phototherapeutic agent administered to the patient to electromagnetic radiation. In an embodiment, the phototherapeutic agent is exposed to a therapeutically effective amount of electromagnetic radiation. As used herein, a therapeutically effective amount of electromagnetic radiation is an amount for achieving a desired therapeutic result, for example an amount for generating a therapeutically effective amount of reactive species for damaging or causing local cell death of a selected target tissue. In an embodiment, the method further comprises generating one or more reactive species from said compound administered to the patient via the exposure of the phototherapeutic agent to applied electromagnetic radiation. In an embodiment, for example, the method further comprises the step of cleaving one or more photolabile C—N, N═N and/or C—X (wherein X is a halo group) bonds so as to generate reactive species, such as free radicals. In an embodiment, the method further comprises contacting a selected organ or selected tissue in the patient with the phototherapeutic agent. In an embodiment, a therapeutically effective dose of the phototherapeutic agent is administered to a patient in need of treatment.

Phototherapeutic agents useful in the present methods include fused ring azo and diaza compounds having a first ring having a intra-ring azo or intra-ring diaza group, wherein the first ring is fused to second unsaturated ring and third aromatic ring. Phototherapeutic agents useful in the present methods include compounds optionally having a targeting ligand for targeted administration. Phototherapeutic agents useful in the present methods include compounds optionally having a dye component, such as a fluorophore or chromophore, for imaging and/or visualization functionality. In an embodiment, the method of the invention comprises administering to a patient a compound having any one of formula (FX1)-(FX40), including any of the specific compositions classes and compounds described in connection with formula (FX1)-(FX40). As will be understood by one of skill in the art, the present methods expressly include methods of using phototherapeutic agents, wherein the phototherapeutic agent includes the compound classes, compounds, and all variations thereof, described herein, including the compound classes, compounds and variations described in connection with any one of formulas (FX1)-(FX40).

Embodiments of this aspect may comprise a method of carrying out an in vivo therapeutic and/or diagnostic procedure. In an embodiment, the invention comprises a method of carrying out an in vivo phototherapeutic, photoactivation, and/or photosensitizing procedure. The present methods have broad clinical utility which includes, but is not limited to, phototherapy of tumors, inflammatory processes, and impaired vasculature. In embodiments, subjects of the invention may be any mammal, such as a human, and optionally the subject of the present methods is a patient in need of treatment and/or diagnosis. The present methods are also useful for in ex vivo and in vitro procedures, including medical therapeutic and diagnostic procedures.

Methods of the invention may optionally further comprise a number of additional steps, processes and/or conditions. In an embodiment, the method further comprises the step of administering the phototherapeutic agent into a bodily fluid of the subject. The phototherapeutic agent may be introduced into the patient by any suitable method, including intravenous, intraperitoneal or subcutaneous injection or infusion, oral administration, transdermal absorption through the skin, or by inhalation. In an embodiment, the method further comprises contacting a target tissue, such as an organ, tissue, tumor, lesion, or cell type, with a compound of any one of formulas (FX1)-(FX40) prior to or during the exposure step. In an embodiment, the method further comprises allowing the compound to accumulate in a target tissue prior to exposure of the phototherapeutic agent to electromagnetic radiation. In an embodiment, the method further comprises contacting and/or selectively targeting the diagnostic agent to a selected organ, tissue, tumor, lesion, inflammation, or cell type. In an embodiment, the phototherapeutic agent is administered to the skin, a tumor, surgical site, or a wound site. In an embodiment, for example, the phototherapeutic agent is administered and/or delivered to a blood vessel, lung, heart, throat, ear, rectum, bladder, stomach, intestines, esophagus, liver, brain, prostrate, breast, or pancreas of the subject.

As will be understood by one having skill in the art, the optical conditions for the step of exposing the phototherapeutic agent administered to the patient to electromagnetic radiation will vary considerably with the (i) therapeutic and/or diagnostic objectives, and (ii) the condition of the subject (e.g., height, weight, state of health etc.). In an embodiment, the applied electromagnetic radiation has wavelengths, energy and/or fluence sufficient to achieve a desired therapeutic and/or diagnostic result. In an embodiment, the electromagnetic radiation has wavelengths, energy and/or fluence sufficient to activate the phototherapeutic agent, for example wavelengths, energy and/or fluence sufficient to result in generation of reactive species, including free radicals and/or optionally singlet oxygen. In an embodiment, the electromagnetic radiation has wavelengths, energy and/or fluence sufficient to result in cleavage of at least one photolabile bond of the optical agent upon absorption and, optionally internal energy transfer process(es). In an embodiment, the electromagnetic radiation exposed to the phototherapeutic agent has wavelengths corresponding to a maximum in the absorption spectrum of the phototherapeutic agent, preferably for some applications a maximum (e.g., within 20 nm of a maximum in the absorption spectrum) in the visible or NIR regions of the electromagnetic spectrum. Optionally, excitation is achieved using electromagnetic radiation substantially free (e.g., less than about 10% of total radiant energy), of ultraviolet radiation, for example, to minimize exposure of the subject to electromagnetic radiation capable of causing unwanted cell or tissue damage. Electromagnetic radiation may be provided to the phototherapeutic agent using a range of optical sources and/or surgical instrumentation, including a laser, electromagnetic radiation emitting diodes, fiber optic device, endoscope, catheter, optical filters, mirrors, lenses, or any combination of these.

### EXAMPLE 3

### Biotargeting Using Fused Ring Azo and Diaza Compounds

Compounds of the invention are also useful for targeting biological moieties. Targeted moieties may also undergo subsequent or coincident phototherapeutic applications.

In aspects of this embodiment, compounds of the formulas (FX1) to (FX40) contain one or more biotargeting groups. These ligands are well known in the art. By way of example, the fused ring azo or diaza compound which includes a targeting moiety can be administered to a patient in a therapeutically or diagnostically effective amount to photoactivate and/or detect the fused ring azo or diaza compound within the patient. After a period of time has lapsed for the compound to bind to, or otherwise associate with, the desired target, the whole body or portion thereof (e.g., site of tumor, lesion or wound) is exposed to electromagnetic radiation of suitable wavelength to photoexcite the fused ring azo or diaza compound. In some methods, photoexcitation at a target tissue initiates localized generation of reactive species for achieving a desired therapeutic outcome. In some embodiments, for example, electromagnetic radiation emanating from the patient as a result of the absorption and excitation of the fused ring azo or diaza compound is then detected. By evaluating the location, intensity, and/or wavelength of electromagnetic radiation emanating from the patient, a diagnosis can be made as a result of the targeting properties of the fused ring azo or diaza compound.

In embodiments, compounds of the invention are useful for both oncology and non-oncology applications. Some specific targets are tumors accessible via endoscope. In an application, a compound that targets a protein, polypeptide, oligonucleotide or other biomolecule associated with such a tumor is administered to the tumor via endoscope or other useful method. Then, the compounds of the invention can be used in phototherapeutic applications or imaging applications. Other specific targets include colon, lung, ovarian, cervical, esophageal, bladder, blood, and stomach cancers; endometriosis, and bacterial infections. Particular targeting groups include ST receptor binding agents, bombesin receptor binding agents, leukemia peptides, and folate receptor binding agents. Some examples of targeting peptides are described in WO/2008/108941.

### EXAMPLE 4

### Pharmaceutical Formulations

### Therapeutically Effective Amount

Toxicity and therapeutic efficacy of such compounds and bioconjugates can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compounds and bioconjugates that exhibit large therapeutic indices are preferred. While compounds and bioconjugates exhibiting toxic side effects may be used, care should be taken to design a delivery system that targets such compounds and bioconjugates to the site affected by the disease or disorder in order to minimize potential damage to unaffected cells and reduce side effects.

The data obtained from the cell culture assays and animal studies can be used in formulating a range of dosages for use in humans and other mammals. The dosage of such compounds and bioconjugates lies preferably within a range of circulating plasma or other bodily fluid concentrations that include the ED₅₀ and provides clinically efficacious results (i.e., reduction in disease symptoms). The dosage may vary within this range depending upon the dosage form employed and the route of administration utilized. For any compound and bioconjugate of the present invention, the therapeutically effective amount can be estimated initially from cell culture assays. A dosage may be formulated in animal models to achieve a circulating plasma concentration range that includes the IC₅₀ (the concentration of the test compound that achieves a half-maximal inhibition of symptoms) as determined in cell culture. Such information can be used to more accurately determine useful dosages in humans and other mammals. Compound and bioconjugate levels in plasma may be measured, for example, by high performance liquid chromatography.

An amount of a compound or bioconjugate that may be combined with a pharmaceutically acceptable carrier to produce a single dosage form will vary depending upon the host treated and the particular mode of administration. It will be appreciated by those skilled in the art that the unit content of a compound/bioconjugate contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage and dosage regime for treating a disease or condition may be selected in accordance with a variety of factors, including the type, age, weight, sex, diet and/or medical condition of the patient, the route of administration, pharmacological considerations such as activity, efficacy, pharmacokinetic and/or toxicology profiles of the particular compound/bioconjugate employed, whether a compound/bioconjugate delivery system is utilized, and/or whether the compound/bioconjugate is administered as a pro-drug or part of a drug combination. Thus, the dosage regime actually employed may vary widely from subject to subject, or disease to disease and different routes of administration may be employed in different clinical settings.

Variations on compositions including salts and ester forms of compounds: Compounds of this invention and compounds useful in the methods of this invention include those of the compounds and formula(s) described herein and pharmaceutically-acceptable salts and esters of those compounds. In embodiments, salts include any salts derived from the acids and bases of the formulas herein which are acceptable for use in human or veterinary applications. In embodiments, the term ester refers to hydrolyzable esters of compounds of the names and formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

In an embodiment, the invention provides a method for treating a medical condition comprising administering to a subject (e.g. patient) in need thereof, a therapeutically effective amount of a composition of the invention, such as a compound of any one of formulas (FX1)-(FX40). In an embodiment, the medical condition is cancer, or various other diseases, injuries, and disorders, including cardiovascular disorders such as atherosclerosis and vascular restenosis, inflammatory diseases, ophthalmic diseases and dermatological diseases.

The identified compounds/bioconjugates treat, inhibit, control and/or prevent, or at least partially arrest or partially prevent, diseases and conditions of interest and can be administered to a subject at therapeutically effective amounts and optionally diagnostically effective amounts. Compositions/formulations of the present invention comprise a therapeutically effective amount (which may optionally include a diagnostically effective amount) of at least one compound or bioconjugate of the present invention. Subjects receiving treatment that includes a compound/bioconjugate of the invention are preferably animals (e.g., mammals, reptiles and/or avians), more preferably humans, horses, cows, dogs, cats, sheep, pigs, and/or chickens, and most preferably humans.

In an embodiment, the invention provides a medicament which comprises a therapeutically effective amount of one or more compositions of the invention, such as a compound of any one of formulas (FX1)-(FX40). In an embodiment, the invention provides a method for making a medicament for treatment of a condition described herein. In an embodiment, the invention provides a method for making a medicament for diagnosis or aiding in the diagnosis of a condition described herein. In an embodiment, the invention provides the use of one or more compositions set forth herein for the making of a medicament.

Compounds of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds set forth herein.

In an embodiment, a composition of the invention is isolated or purified. In an embodiment, an isolated or purified compound may be at least partially isolated or purified as would be understood in the art. In an embodiment, the composition of the invention has a chemical purity of 95%, optionally for some applications 99%, optionally for some applications 99.9%, optionally for some applications 99.99% pure, and optionally for some applications 99.999% pure.

Typically, a compound of the invention, or pharmaceutically acceptable salt thereof, is administered to a subject in a diagnostically or therapeutically effective amount. One skilled in the art generally can determine an appropriate dosage. Factors affecting a particular dosage regimen (including the amount of compound delivered, frequency of administration, and whether administration is continuous or intermittent) include, for example, the type, age, weight, sex, diet, and condition of the subject; the type of pathological condition and its severity; and the nature of the desired therapeutic effect. Pharmacological considerations include fused ring azo or diaza compound activity, efficacy, pharmacokinetic, and toxicology profiles of the particular fused ring azo or diaza compound used; the route of administration and whether a drug delivery system is utilized; and whether the fused ring azo or diaza compound is administered as part of a combination therapy (e.g., whether the agent is administered in combination with one or more other active compounds, other agents, radiation, and the like).

Compositions for oral administration may be, for example, prepared in a manner such that a single dose in one or more oral preparations contains at least about 20 mg of the fused ring azo or diaza compound per square meter of subject body surface area, or at least about 50, 100, 150, 200, 300, 400, or 500 mg of the fused ring azo or diaza compound per square meter of subject body surface area (the average body surface area for a human is, for example, 1.8 square meters). In particular, a single dose of a composition for oral administration can contain from about 20 to about 600 mg, and in certain aspects from about 20 to about 400 mg, in another aspect from about 20 to about 300 mg, and in yet another aspect from about 20 to about 200 mg of the fused ring azo or diaza compound per square meter of subject body surface area. Compositions for parenteral administration can be prepared in a manner such that a single dose contains at least about 20 mg of the fused ring azo or diaza compound per square meter of subject body surface area, or at least about 40, 50, 100, 150, 200, 300, 400, or 500 mg of the fused ring azo or diaza compound per square meter of subject body surface area. In particular, a single dose in one or more parenteral preparations contains from about 20 to about 500 mg, and in certain aspects from about 20 to about 400 mg, and in another aspect from about 20 to about 450 mg, and in yet another aspect from about 20 to about 350 mg of the fused ring azo or diaza compound per square meter of subject body surface area. It should be recognized that these oral and parenteral dosage ranges represent generally preferred dosage ranges, and are not intended to limit the invention. The dosage regimen actually employed can vary widely, and, therefore, can deviate from the generally preferred dosage regimen. It is contemplated that one skilled in the art will tailor these ranges to the individual subject.

As indicated above, it is contemplated that the compounds and pharmaceutically acceptable salts of the invention may be used as part of a combination. The term “combination” means the administration of two or more compounds directed to the target condition. The treatments of the combination generally may be co-administered in a simultaneous manner. Two compounds can be co-administered as, for example: (a) a single formulation (e.g., a single capsule) having a fixed ratio of active ingredients; or (b) multiple, separate formulations (e.g., multiple capsules) for each compound. The treatments of the combination may alternatively (or additionally) be administered at different times.

It is further contemplated that the fused ring azo and diaza compounds and salts of this invention can be used in the form of a kit that is suitable for use in performing the methods described herein, packaged in a container. The kit can contain the fused ring azo or diaza compound or compounds and, optionally, appropriate diluents, devices or device components suitable for administration and instructions for use in accordance with the methods of the invention. The devices can include parenteral injection devices, such as syringes or transdermal patch or the like. Device components can include cartridges for use in injection devices and the like. In one aspect, the kit includes a first dosage form including a fused ring azo or diaza compound or salt of this invention and a second dosage form including another active ingredient in quantities sufficient to carry out the methods of the invention. The first dosage form and the second dosage form together can include a therapeutically effective amount of the compounds for treating the targeted condition(s).

This invention also is directed, in part, to pharmaceutical compositions including a therapeutically effective amount of a compound or salt of this invention, as well as processes for making such compositions. Such compositions generally include one or more pharmaceutically acceptable carriers (e.g., excipients, vehicles, auxiliaries, adjuvants, diluents) and may include other active ingredients. Formulation of these compositions may be achieved by various methods known in the art. A general discussion of these methods may be found in, for example, Hoover, John E., Remington's Pharmaceutical Sciences (Mack Publishing Co., Easton, Pa.: 1975). See also, Lachman, L., eds., Pharmaceutical Dosage Forms (Marcel Decker, New York, N.Y., 1980).

The preferred composition depends on the route of administration. Any route of administration may be used as long as the target of the compound or pharmaceutically acceptable salt is available via that route. Suitable routes of administration include, for example, oral, intravenous, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

Pharmaceutically acceptable carriers that may be used in conjunction with the compounds of the invention are well known to those of ordinary skill in the art. Carriers can be selected based on a number of factors including, for example, the particular fused ring azo or diaza compound(s) or pharmaceutically acceptable salt(s) used; the compound's concentration, stability, and intended bioavailability; the condition being treated; the subject's age, size, and general condition; the route of administration; etc. A general discussion related to carriers may be found in, for example, J. G. Nairn, Remington's Pharmaceutical Science, pp. 1492-1517 (A. Gennaro, ed., Mack Publishing Co., Easton, Pa. (1985)).

Solid dosage forms for oral administration include, for example, capsules, tablets, gelcaps, pills, dragees, troches, powders, granules, and lozenges. In such solid dosage forms, the compounds or pharmaceutically acceptable salts thereof can be combined with one or more pharmaceutically acceptable carriers. The compounds and pharmaceutically acceptable salts thereof can be mixed with carriers including, but not limited to, lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can include buffering agents, such as sodium citrate, or magnesium or calcium carbonate or bicarbonate. Tablets and pills additionally can, for example, include a coating (e.g., an enteric coating) to delay disintegration and absorption. The concentration of the fused ring azo or diaza compound in a solid oral dosage form can be from about 5 to about 50%, and in certain aspects from about 8 to about 40%, and in another aspect from about 10 to about 30% by weight based on the total weight of the composition.

Liquid dosage forms of the compounds of the invention for oral administration include, for example, pharmaceutically acceptable emulsions, solutions, suspensions, syrups, and elixirs containing inert diluents commonly used in the art (e.g., water). Such compositions also can include adjuvants, such as wetting, emulsifying, suspending, flavoring (e.g., sweetening), and/or perfuming agents. The concentration of the fused ring azo or diaza compound in the liquid dosage form can be from about 0.01 to about 5 mg, and in certain aspects from about 0,01 to about 1 mg, and in another aspect from about 0.01 to about 0.5 mg per ml of the composition. Low concentrations of the compounds of the invention in liquid dosage form can be prepared in the case that the fused ring azo or diaza compound is more soluble at low concentrations. Techniques for making oral dosage forms useful in the invention are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

In some aspects of the invention, tablets or powders for oral administration can be prepared by dissolving the fused ring azo or diaza compound in a pharmaceutically acceptable solvent capable of dissolving the compound to form a solution and then evaporating when the solution is dried under vacuum. A carrier can also be added to the solution before drying. The resulting solution can be dried under vacuum to form a glass. The glass can then mix with a binder to form a powder. This powder may be mixed with fillers or other conventional tableting agents, and then processed to form a tablet. Alternatively, the powder may be added to a liquid carrier to form a solution, emulsion, suspension, or the like.

In some aspects, solutions for oral administration are prepared by dissolving the fused ring azo or diaza compound in a pharmaceutically acceptable solvent capable of dissolving the compound to form a solution. An appropriate volume of a carrier is added to the solution while stirring to form a pharmaceutically acceptable solution for oral administration.

“Parenteral administration” includes subcutaneous injections, intravenous injections, intraarterial injections, intraorbital injections, intracapsular injections, intraspinal injections, intraperitoneal injections, intramuscular injections, intrasternal injections, and infusion. Dosage forms suitable for parenteral administration include solutions, suspensions, dispersions, emulsions, and any other dosage form that can be administered parenterally.

Injectable preparations (e.g., sterile injectable aqueous or oleaginous suspensions) can be formulated according to the known art using suitable dispersing, wetting agents, and/or suspending agents. Acceptable vehicles for parenteral use include both aqueous and nonaqueous pharmaceutically-acceptable solvents. Suitable pharmaceutically acceptable aqueous solvents include, for example, water, saline solutions, dextrose solutions (e.g., such as DW5), electrolyte solutions, etc.

In one embodiment, the present fused ring azo and diaza compounds are formulated as nanoparticles or microparticles. Use of such nanoparticle or microparticle formulations may be beneficial for some applications to enhance delivery, localization, target specificity, administration, etc. of the fused ring azo or diaza compound. Potentially useful nanoparticles and microparticles include, but are not limited to, micelles, liposomes, microemulsions, nanoemulsions, vesicles, tubular micelles, cylindrical micelles, bilayers, folded sheets structures, globular aggregates, swollen micelles, inclusion complex, encapsulated droplets, microcapsules, nanocapsules or the like. As will be understood by those having skill in the art, the present fused ring azo and diaza compounds can be located inside the nanoparticle or microparticle, within a membrane or wall of the nanoparticle or microparticle, or outside of (but bonded to or otherwise associated with) the nanoparticle or microparticle. The agent formulated in nanoparticles or microparticles may be administered by any of the routes previously described. In a formulation applied topically, the fused ring azo or diaza compound is slowly released over time. In an injectable formulation, the liposome, micelle, capsule, etc., circulates in the bloodstream and is delivered to the desired site (e.g., target tissue).

Preparation and loading of nanoparticles and microparticles are well known in the art. As one example, liposomes may be prepared from dipalmitoyl phosphatidylcholine (DPPC) or egg phosphatidylcholine (PC) because this lipid has a low heat transition. Liposomes are made using standard procedures as known to one skilled in the art (e.g., Braun-Falco et al., (Eds.), Griesbach Conference, Liposome Dermatics, Springer-Verlag, Berlin (1992), pp. 69 81; 91 117 which is expressly incorporated by reference herein). Polycaprolactone, poly(glycolic) acid, poly(lactic) acid, polyanhydride or lipids may be formulated as microspheres. As an illustrative example, the present fused ring azo and diaza compounds may be mixed with polyvinyl alcohol (PVA), the mixture then dried and coated with ethylene vinyl acetate, then cooled again with PVA. In a liposome, the present fused ring azo and diaza compounds may be within one or both lipid bilayers, in the aqueous between the bilayers, or within the center or core. Liposomes may be modified with other molecules and lipids to form a cationic liposome. Liposomes may also be modified with lipids to render their surface more hydrophilic which increases their circulation time in the bloodstream. The thus-modified liposome has been termed a “stealth” liposome, or a long-lived liposome, as described in U.S. Pat. No. 6,258,378, and in Stealth Liposomes, Lasic and Martin (Eds.) 1995 CRC Press, London, which are expressly incorporated by reference herein. Encapsulation methods include detergent dialysis, freeze drying, film forming, injection, as known to one skilled in the art and disclosed in, for example, U.S. Pat. No. 6,406,713 which is expressly incorporated by reference herein in its entirety. Optionally, the present compositions and methods include a micelle delivery system, for example, involving one or more PEG-based amphiphilic polymers developed for drug delivery including PEG-poly(ε-caprolactone), PEG-poly(amino acid), PEG-polylactide or a PEG-phospholid constructs; a cross linked poly(acrylic acid) polymer system, a phospholipid-based system and/or block copolymer systems comprising one or more of the following polymer blocks a poly(lactic acid) polymer block, a poly(propylene glycol) polymer block; a poly(amino acid) polymer block; a poly(ester) polymer block; and a poly(ε-caprolactone) polymer block, a poly(ethylene glycol) block, a poly(acrylic acid) block, a polylactide block, a polyester block, a polyamide block, a polyanhydride block, a polyurethane block, a polyimine block, a polyurea block, a polyacetal block, a polysaccharide block and a polysiloxane block.

Suitable pharmaceutically-acceptable nonaqueous solvents include, but are not limited to, the following (as well as mixtures thereof): alcohols (these include, for example, σ-glycerol formal, β-glycerol formal, 1,3-butyleneglycol, aliphatic or aromatic alcohols having from 2 to about 30 carbons (e.g., methanol, ethanol, propanol, isopropanol, butanol, t-butanol, hexanol, octanol, amylene hydrate, benzyl alcohol, glycerin (glycerol), glycol, hexylene, glycol, tetrahydrofuranyl alcohol, cetyl alcohol, and stearyl alcohol), fatty acid esters of fatty alcohols (e.g., polyalkylene glycols, such as polypropylene glycol and polyethylene glycol), sorbitan, sucrose, and cholesterol); amides (these include, for example, dimethylacetamide (DMA), benzyl benzoate DMA, dimethylformamide, N-hydroxyethyO-lactamide, N,N-dimethylacetamide-amides, 2-pyrrolidinone, 1-methyl-2-pyrrolidinone, and polyvinylpyrrolidone); esters (these include, for example, acetate esters (e.g., monoacetin, diacetin, and triacetin), aliphatic and aromatic esters (e.g., ethyl caprylate or octanoate, alkyl oleate, benzyl benzoate, or benzyl acetate), dimethylsulfoxide (DMSO), esters of glycerin (e.g., mono, di, and tri-glyceryl citrates and tartrates), ethyl benzoate, ethyl acetate, ethyl carbonate, ethyl lactate, ethyl oleate, fatty acid esters of sorbitan, glyceryl monostearate, glyceride esters (e.g., mono, di, or tri-glycerides), fatty acid esters (e.g., isopropyl myristrate), fatty acid derived PEG esters (e.g., PEG-hydroxyoleate and PEG-hydroxystearate), N-methyl pyrrolidinone, pluronic 60, polyoxyethylene sorbitol oleic polyesters (e.g., poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate), polyoxyethylene sorbitan esters (e.g., polyoxyethylene-sorbitan monooleate, polyoxyethylene-sorbitan monopalmitate, polyoxyethylene-sorbitan monolaurate, polyoxyethylene-sorbitan monostearate, and POLYSORBATE 20, 40, 60, and 80 (from ICI Americas, Wilmington, Del.)), polyvinylpyrrolidone, alkyleneoxy modified fatty acid esters (e.g., polyoxyl 40 hydrogenated castor oil and polyoxyethylated castor oils, such as CREMOPHOR EL solution or CREMOPHOR RH 40 solution), saccharide fatty acid esters (i.e., the condensation product of a monosaccharide (e.g., pentoses, such as, ribose, ribulose, arabinose, xylose, lyxose, and xylulose; hexoses, such as glucose, fructose, galactose, mannose, and sorbose; trioses; tetroses; heptoses; and octoses), disaccharide (e.g., sucrose, maltose, lactose, and trehalose), oligosaccharide, or a mixture thereof with one or more C₄-C₂₂ fatty acids (e.g., saturated fatty acids, such as caprylic acid, capric acid, lauric acid, myristic acid, palmitic acid, and stearic acid; and unsaturated fatty acids, such as palmitoleic acid, oleic acid, elaidic acid, erucic acid, and linoleic acid), and steroidal esters); ethers (these are typically alkyl, aryl, and cyclic ethers having from 2 to about 30 carbons. Examples include diethyl ether, tetrahydrofuran, dimethyl isosorbide, diethylene glycol monoethyl ether), and glycofurol (tetrahydrofurfuranyl alcohol polyethylene glycol ether); ketones (these typically have from about 3 to about 30 carbons. Examples include acetone, methyl ethyl ketone, methyl isobutyl ketone); hydrocarbons (these are typically aliphatic, cycloaliphatic, and aromatic hydrocarbons having from about 4 to about 30 carbons). Examples include benzene, cyclohexane, dichloromethane, dioxolanes, hexane, n-decane, n-dodecane, n-hexane, sulfolane, tetramethylenesulfone, tetramethylenesulfoxide, toluene, dimethylsulfoxide (DMSO); and tetramethylene sulfoxide; oils (these include oils of mineral, vegetable, animal, essential, or synthetic origin). These include mineral oils, such as aliphatic and wax-based hydrocarbons, aromatic hydrocarbons, mixed aliphatic and aromatic based hydrocarbons, and refined paraffin oil; vegetable oils, such as linseed, tung, safflower, soybean, castor, cottonseed, groundnut, rapeseed, coconut, palm, olive, corn, corn germ, sesame, persic, and peanut oil; glycerides, such as mono-, di-, and triglycerides; animal oils, such as fish, marine, sperm, cod-liver, haliver, squaiene, squalane, and shark liver oil; oleic oils; and polyoxyethylated castor oil); alkyl, alkenyl, or aryl halides (these include alkyl or aryl halides having from 1 to about 30 carbons and one or more halogen substituents. Examples include methylene chloride); monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (SOLUTOL HS-15, from BASF, Ludwigshafen, Germany); polyoxyethylene glycerol; sodium laurate; sodium oleate; and sorbitan monooleate. Other pharmaceutically acceptable solvents for use in the invention are well known to those of ordinary skill in the art. General discussion relating to such solvents may be found in, for example, The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics 3d ed., (G. Banker et. al., eds., Marcel Dekker, Inc., New York, N.Y. (1995)), The Pharmacological Basis of Therapeutics, (Goodman & Gilman, McGraw Hill Publishing), Pharmaceutical Dosage Forms, (H. Lieberman et. al., eds., Marcel Dekker, Inc., New York, N.Y. (1980)), Remington's Pharmaceutical Sciences, 19th ed., (A. Gennaro, ed., Mack Publishing, Easton, Pa., (1995)), The United States Pharmacopeia 24, The National Formulary 19, (National Publishing, Philadelphia, Pa. (2000)); Spiegel, A. J., et al., “Use of Nonaqueous Solvents in Parenteral Products,” J. Pharma. Sciences, Vol. 52, No. 10, pp. 917-927 (1963),

Solvents useful in the invention include, but are not limited to, those known to stabilize the fused ring azo and diaza compounds or pharmaceutically acceptable salts thereof. These typically include, for example, oils rich in triglycerides, such as safflower oil, soybean oil, and mixtures thereof; and alkyleneoxy-modified fatty acid esters, such as polyoxyl 40 hydrogenated castor oil and polyoxyethylated castor oils (e.g., CREMOPHOR EL solution or CREMOPHOR RH 40 solution). Commercially available triglycerides include INTRALIPID emulsified soybean oil (Kabi-Pharmacia Inc., Stockholm, Sweden), NUTRALIPID emulsion (McGaw, Irvine, Calif.), LIPOSYN II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), LIPOSYN III 2% emulsion (a 2% fat emulsion solution containing 100 mg safflower oil, 100 mg soybean oil, 12 mg egg phosphatides, and 25 mg glycerin per ml of solution; Abbott Laboratories, Chicago, Ill.), natural or synthetic glycerol derivatives containing the docosahexaenoyl group at levels of from about 25 to about 100% (by weight based on the total fatty acid content) (DHASCO from Martek Biosciences Corp., Columbia, Md.; DHA MAGURO from Daito Enterprises, Los Angeles, Calif.; SOYACAL; and TRAVEMULSION). Ethanol in particular is a useful solvent for dissolving a fused ring azo or diaza compound or pharmaceutically acceptable salt thereof to form solutions, emulsions, and the like.

Additional components can be included in the compositions of this invention for various purposes generally known in the pharmaceutical industry. These components tend to impart properties that, for example, enhance retention of the fused ring azo or diaza compound or salt at the site of administration, protect the stability of the composition, control the pH, and facilitate processing of the fused ring azo or diaza compound or salt into pharmaceutical formulations, and the like. Specific examples of such components include cryoprotective agents; agents for preventing reprecipitation of the fused ring azo or diaza compound or salt surface; active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stearyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide, etc.); and antioxidants (e.g., sodium metabisulfite, sodium bisulfite, sodium sulfite, dextrose, phenols, thiophenols, etc.). Techniques and compositions for making parenteral dosage forms are generally known in the art. Formulations for parenteral administration may be prepared from one or more sterile powders and/or granules having a compound or salt of this invention and one or more of the carriers or diluents mentioned for use in the formulations for oral administration. The powder or granule typically is added to an appropriate volume of a solvent (typically while agitating (e.g., stirring) the solvent) that is capable of dissolving the powder or granule. Particular solvents useful in the invention include, for example, water, polyethylene glycol, propylene glycol, ethanol, corn oil, cottonseed oil, peanut oil, sesame oil, benzyl alcohol, sodium chloride, and/or various buffers.

Emulsions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the emulsion. Solutions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the solution.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, cocoa butter; synthetic mono-, di-, or triglycerides; fatty acids; and/or polyethylene glycols.

“Topical administration” includes the use of transdermal administration, such as transdermal patches or iontophoresis devices.

If desired, the emulsions or solutions described above for oral or parenteral administration can be packaged in IV bags, vials, or other conventional containers in concentrated form, and then diluted with a pharmaceutically acceptable liquid (e.g., saline) to form an acceptable fused ring azo or diaza compound concentration before use.

Other adjuvants and modes of administration well known in the pharmaceutical art may also be used. Pharmaceutically acceptable salts comprise pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br″), sulfate, acetates (e.g., acetate, trifluoroacetate), ascorbates, aspartates, benzoates, citrates, and lactate.

It is understood that this invention is not limited to the particular compounds, methodology, protocols, and reagents described, as these may vary. It is also to be understood that the terminology used herein is for the purpose of describing particular embodiments only, and is not intended to limit the scope of the invention which will be limited only by the appended claims.

Compositions of the invention includes formulations and preparations comprising one or more of the present compounds provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

### Formulations and Use

Compounds and bioconjugates of the present invention may be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. An individual compound/bioconjugate may be administered in combination with one or more additional compounds/bioconjugates of the present invention and/or together with other biologically active or biologically inert agents. Such biologically active or inert agents may be in fluid or mechanical communication with the compound(s)/bioconjugate(s) or attached to the compound(s)/bioconjugate(s) by ionic, covalent, Van der Waals, hydrophobic, hydrophilic or other physical forces. It is preferred that administration is localized in a subject, but administration may also be systemic.

Compounds and bioconjugates of the present invention may be formulated by any conventional manner using one or more pharmaceutically acceptable carriers. Thus, the compounds/bioconjugates and their pharmaceutically acceptable salts and solvates may be specifically formulated for administration, e.g., by inhalation or insufflation (either through the mouth or the nose) or oral, buccal, parenteral or rectal administration. The compounds/bioconjugates may take the form of charged, neutral and/or other pharmaceutically acceptable salt forms. Examples of pharmaceutically acceptable carriers include, but are not limited to, those described in REMINGTON'S PHARMACEUTICAL SCIENCES (A. R. Gennaro, Ed.), 20th edition, Williams & Wilkins Pa., USA (2000).

Compounds and bioconjugates of the present invention may be formulated in the form of solutions, suspensions, emulsions, tablets, pills, capsules, powders, controlled- or sustained-release formulations and the like. Such formulations will contain a therapeutically effective amount of the compound/bioconjugate, preferably in purified form, together with a suitable amount of carrier so as to provide the form for proper administration to the patient. The formulation should suit the mode of administration.

### Parenteral Administration

Compounds and bioconjugates of the present invention may be formulated for parenteral administration by injection (e.g., by bolus injection or continuous infusion). Formulations for injection may be presented in unit dosage form in ampoules or in multi-dose containers with an optional preservative added. The parenteral preparation can be enclosed in ampoules, disposable syringes or multiple dose vials made of glass, plastic or the like. The formulation may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the parenteral preparation.

Alternatively, compounds and bioconjugates of the present invention may be formulated in powder form for constitution with a suitable vehicle, such as sterile pyrogen-free water, before use. For example, a compound/bioconjugate suitable for parenteral administration may include a sterile isotonic saline solution containing between 0.1 percent and 90 percent weight per volume of the compound/bioconjugate. By way of example, a solution may contain from about 5 percent to about 20 percent, more preferably from about 5 percent to about 17 percent, more preferably from about 8 to about 14 percent, and still more preferably about 10 percent of the compound/bioconjugate. The solution or powder preparation may also include a solubilizing agent and a local anesthetic such as lignocaine to ease pain at the site of the injection. Other methods of parenteral delivery of compounds/bioconjugates will be known to the skilled artisan and are within the scope of the invention.

### Oral Administration

For oral administration, a compound/bioconjugate of the invention may be formulated to take the form of tablets or capsules prepared by conventional means with one or more pharmaceutically acceptable carriers (e.g., excipients such as binding agents, fillers, lubricants and disintegrants):

### A. Binding Agents

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation.

### B. Fillers

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

### C. Lubricants

Lubricants include, but are not limited to, calcium stearate, magnesium stearate, mineral oil, electromagnetic radiation mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

### D. Disintegrants

Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

The tablets or capsules may optionally be coated by methods well known in the art. If binders and/or fillers are used with a compound/bioconjugate of the invention, they are typically formulated as about 50 to about 99 weight percent of the compound/bioconjugate. In one aspect, about 0.5 to about 15 weight percent of disintegrant, and particularly about 1 to about 5 weight percent of disintegrant, may be used in combination with the compound. A lubricant may optionally be added, typically in an amount of less than about 1 weight percent of the compound/bioconjugate. Techniques and pharmaceutically acceptable additives for making solid oral dosage forms are described in Marshall, SOLID ORAL DOSAGE FORMS, Modern Pharmaceutics (Banker and Rhodes, Eds.), 7:359-427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may also be formulated to achieve controlled release of the compound/bioconjugate. Oral formulations preferably contain 10% to 95% compound/bioconjugate. In addition, a compound/bioconjugate of the present invention may be formulated for buccal administration in the form of tablets or lozenges formulated in a conventional manner. Other methods of oral delivery of compounds/bioconjugates of the invention will be known to the skilled artisan and are within the scope of the invention.

### Controlled-Release Administration

Controlled-release (or sustained-release) preparations may be formulated to extend the activity of a compound/bioconjugate and reduce dosage frequency. Controlled-release preparations can also be used to effect the time of onset of action or other characteristics, such as blood levels of the compound/bioconjugate, and consequently affect the occurrence of side effects.

Controlled-release preparations may be designed to initially release an amount of a compound/bioconjugate that produces the desired therapeutic effect, and gradually and continually release other amounts of the compound/bioconjugate to maintain the level of therapeutic effect over an extended period of time. In order to maintain a near-constant level of a compound/bioconjugate in the body, the compound/bioconjugate can be released from the dosage form at a rate that will replace the amount of compound/bioconjugate being metabolized and/or excreted from the body. The controlled-release of a compound/bioconjugate may be stimulated by various inducers, e.g., change in pH, change in temperature, enzymes, water, and/or other physiological conditions or molecules.

Controlled-release systems may include, for example, an infusion pump which may be used to administer the compound/bioconjugate in a manner similar to that used for delivering insulin or chemotherapy to the body generally, or to specific organs or tumors. Typically, using such a system, the compound/bioconjugate is administered in combination with a biodegradable, biocompatible polymeric implant that releases the compound/bioconjugate over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target (e.g., organ, tissue, or group of cells), thus requiring only a fraction of a systemic dosage.

Compounds/bioconjugates of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compounds/bioconjugates will be known to the skilled artisan and are within the scope of the invention.

### Inhalation Administration

Compounds/bioconjugates of the invention may be administered directly to the lung of a patient/subject by inhalation. For administration by inhalation, a compound/bioconjugate may be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas may be used to deliver a compound/bioconjugate directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device may be used to administer a compound/bioconjugate to the lung. DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound/bioconjugate and a suitable powder base such as lactose or starch for these systems.

Another type of device that may be used to deliver a compound/bioconjugate to the lung is a liquid spray device supplied, for example, by Aradigm Corporation. Liquid spray systems use extremely small nozzle holes to aerosolize liquid compound/bioconjugate formulations that may then be directly inhaled into the lung. For example, a nebulizer device may be used to deliver a compound/bioconjugate to the lung. Nebulizers create aerosols from liquid compound/bioconjugate formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another example, an electrohydrodynamic (“EHD”) aerosol device may be used to deliver a compound/bioconjugate to the lung. END aerosol devices use electrical energy to aerosolize liquid compound/bioconjugate solutions or suspensions. The electrochemical properties of the compound/bioconjugate formulation are important parameters to optimize when delivering this compound/bioconjugate to the lung with an EHD aerosol device. Such optimization is routinely performed by one of skill in the art. Other methods of intra-pulmonary delivery of compounds/bioconjugates will be known to the skilled artisan and are within the scope of the invention.

Liquid compound/bioconjugate formulations suitable for use with nebulizers and liquid spray devices and EHD aerosol devices will typically include the compound/bioconjugate with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the compound/bioconjugate. For example, this material may be a liquid such as an alcohol, glycol, polyglycol or a fatty acid. Other methods of formulating liquid compound/bioconjugate solutions or suspensions suitable for use in aerosol devices are known to those of skill in the art.

### Depot Administration

A compound/bioconjugate of the invention may be formulated as a depot preparation. Such long-acting formulations may be administered by implantation (e.g., subcutaneously or intramuscularly) or by intramuscular injection. Accordingly, the compound/bioconjugate may be formulated with suitable polymeric or hydrophobic materials such as an emulsion in an acceptable oil or ion exchange resins, or as sparingly soluble derivatives such as a sparingly soluble salt. Other methods of depot delivery of compounds/bioconjugates will be known to the skilled artisan and are within the scope of the invention.

### Topical Administration

For topical application, a compound/bioconjugate may be combined with a pharmaceutically acceptable carrier so that an effective dosage is delivered, based on the desired activity ranging from an effective dosage, for example, of 1.0 μM to 1.0 mM. In one aspect of the invention, a topical formulation of a compound/bioconjugate can be applied to the skin. The pharmaceutically acceptable carrier may be in the form of, for example, and not by way of limitation, an ointment, cream, gel, paste, foam, aerosol, suppository, pad or gelled stick.

A topical formulation may include a therapeutically effective amount of a compound/bioconjugate in an ophthalmologically acceptable excipient such as buffered saline, mineral oil, vegetable oils such as corn or arachis oil, petroleum jelly, Miglyol 182, alcohol solutions, or liposomes or liposome-like products. Any of these formulations of such compounds/bioconjugates may include preservatives, antioxidants, antibiotics, immunosuppressants, and other biologically or pharmaceutically effective agents that do not exert a significant detrimental effect on the compound/bioconjugate. Other methods of topical delivery of compounds/bioconjugates will be known to the skilled artisan and are within the scope of the invention.

### Rectal Administration

Compounds/bioconjugates of the invention may be formulated in rectal formulations such as suppositories or retention enemas that include conventional suppository bases such as cocoa butter or other glycerides and/or binders and/or carriers such as triglycerides, microcrystalline cellulose, gum tragacanth or gelatin. Rectal formulations can contain a compound/bioconjugate in the range of 0.5% to 10% by weight. Other methods of rectal delivery of compounds/bioconjugates will be known to the skilled artisan and are within the scope of the invention.

### Other Systems of Administration

Various other delivery systems are known in the art and can be used to administer the compounds/bioconjugates of the invention. Moreover, these and other delivery systems may be combined and/or modified to promote optimization of the administration of compounds/bioconjugates of the present invention. Exemplary formulations that include compounds/bioconjugates of the present invention are described below (the compounds/bioconjugates of the present invention are indicated as the active ingredient, but those of skill in the art will recognize that pro-drugs and compound combinations are also meant to be encompassed by this term):

### Formulation 1

Hard gelatin capsules are prepared using the following ingredients:

TABLE 1

| Ingredients | (mg/capsule) |
|---|---|
| Active Ingredient | 250.0 |
| Starch | 305.0 |
| Magnesium stearate | 5.0 |

The above ingredients are mixed and filled into hard gelatin capsules in 560 mg quantities.

### Formulation 2

A tablet formula is prepared using the following ingredients:

TABLE 2

| Ingredients | (mg/tablet) |
|---|---|
| Active Ingredient | 250.0 |
| Cellulose, microcrystalline | 400.0 |
| Colloidal silicon dioxide | 10.0 |
| Stearic acid | 5.0 |

The components are blended and compressed to form tablets, each weighing 665 mg.

### Formulation 3

A dry powder inhaler formulation is prepared containing the following components:

TABLE 3

| Ingredients | Weight % |
|---|---|
| Active ingredient | 5 |
| Lactose | 95 |

The active ingredient is mixed with the lactose and the mixture is added to a dry powder inhaling appliance.

### Formulation 4

Tablets, each containing 60 mg of active ingredient, are prepared as follows:

TABLE 4

| Ingredients | milligrams |
|---|---|
| Active ingredient | 60.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |
| Total | 150.0 |

The active ingredient, starch and cellulose are passed through a No. 20 mesh U.S. sieve and mixed thoroughly. The solution of polyvinylpyrrolidone is mixed with the resultant powders which are then passed through a 16 mesh U.S. sieve. The granules as produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### Formulation 5

Capsules, each containing 80 mg of active ingredient are made as follows:

TABLE 5

| Ingredients | milligrams |
|---|---|
| Active ingredient | 80.0 |
| Starch | 109.0 |
| Magnesium stearate | 1.0 |
| Total | 190.0 |

The active ingredient, cellulose, starch, and magnesium stearate are blended, passed through a No. 20 mesh U.S. sieve, and filled into hard gelatin capsules in 190 mg quantities.

### Formulation 6

Suppositories, each containing 225 mg of active ingredient, are made as follows:

TABLE 6

| Ingredients | milligrams |
|---|---|
| Active Ingredient | 225 |
| Saturated fatty acid glycerides to | 2000 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation 7

Suspensions, each containing 50 mg of active ingredient per 5.0 ml dose are made as follows:

TABLE 7

| Ingredients | milligrams | |
|---|---|---|
| Active ingredient | 50.0 | mg |
| Xanthan gum | 4.0 | mg |
| Sodium carboxymethyl cellulose | (11%) | |
| Microcrystalline cellulose | (89%) 50.0 | mg |
| Sucrose | 1.75 | g |
| Sodium benzoate | 10.0 | mg |
| Flavor | q.v. | |
| Color | q.v. | |
| Purified water to | 5.0 | ml |

The active ingredient, sucrose and xantham gum are blended, passed through a No. 10 mesh U.S. sieve, and mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation 8

Capsules, each containing 150 mg of active ingredient, are made as follows:

TABLE 8

| Ingredients | milligrams |
|---|---|
| Active ingredient | 150.0 |
| Starch | 407.0 |
| Magnesium stearate | 3.0 |
| Total | 560.0 |

The active ingredient, cellulose, starch, and magnesium stearate are blended, passed through a No. 20 mesh U.S. sieve, and filled into hard gelatin capsules in 560 mg quantities.

### Kits

Various embodiments of the present invention include kits. Such kits can include a compound/bioconjugate of the present invention, optionally one or more ingredients for preparing a pharmaceutically acceptable formulation of the compound/bioconjugate, and instructions for use (e.g., administration). When supplied as a kit, different components of a compound/bioconjugate formulation can be packaged in separate containers and admixed immediately before use. Such packaging of the components separately can, if desired, be presented in a pack or dispenser device which may contain one or more unit dosage forms containing the compound/bioconjugate. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one combination for administration together.

Kits may include reagents in separate containers such as, for example, sterile water or saline to be added to a lyophilized active component packaged separately. For example, sealed glass ampules may contain lyophilized superoxide dismutase mimetics and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit, or supplied as electronic mail.

### Statements Regarding Incorporation by Reference and Variations

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the invention and it will be apparent to one skilled in the art that the invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Optical agents of the invention may be formulated with pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R¹)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the invention, the preferred methods and materials are now described. Nothing herein is to be construed as an admission that the invention is not entitled to antedate such disclosure by virtue of prior invention.

In some embodiments, a liposome or micelle may be utilized as a carrier or vehicle for the composition. For example, in some embodiments, the fused ring azo or diaza compound may be a part of the lipophilic bilayers or micelle, and the targeting ligand, if present, may be on the external surface of the liposome or micelle. As another example, a targeting ligand may be externally attached to the liposome or micelle after formulation for targeting the liposome or micelle (which contains the fused ring azo or diaza optical agents) to the desired tissue, organ, or other site in the body.

Every formulation or combination of components described or exemplified herein can be used to practice the invention, unless otherwise stated.

The present compositions, preparations and formulations can be used both as a diagnostic agent as well as a phototherapy agent concomitantly. For example, an effective amount of the present compositions, preparations and formulations in a pharmaceutically acceptable formulation is administered to a patient. Administration is followed by a procedure that combines photodiagnosis and phototherapy. For example, a composition comprising compounds for combined photodiagnosis and phototherapy is administered to a patient and its concentration, localization, or other parameters is determined at the target site of interest. More than one measurement may be taken to determine the location of the target site. The time it takes for the compound to accumulate at the target site depends upon factors such as pharmcokinetics, and may range from about thirty minutes to two days. Once the site is identified, the phototherapeutic part of the procedure may be done either immediately after determining the site or before the agent is cleared from the site. Clearance depends upon factors such as pharmacokinetics.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations may also include aerosol formulation, creams, gels, solutions, etc. The present compositions, preparations and formulations are administered in doses effective to achieve the desired diagnostic and/or therapeutic effect. Such doses may vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations may also optionally include stabilizing agents and skin penetration enhancing agents.

Methods of this invention comprise the step of administering an “effective amount” of the present diagnostic and therapeutic compositions, formulations and preparations containing the present compounds, to diagnosis, image, monitor, evaluate, treat, reduce, alleviate, ameliorate or regulate a biological condition and/or disease state in a patient. The term “effective amount,” as used herein, refers to the amount of the diagnostic and therapeutic formulation, that, when administered to the individual is effective diagnosis, image, monitor, evaluate, treat, reduce alleviate, ameliorate or regulate a biological condition and/or disease state. As is understood in the art, the effective amount of a given composition or formulation will depend at least in part upon, the mode of administration (e.g. intravenous, oral, topical administration), any carrier or vehicle employed, and the specific individual to whom the formulation is to be administered (age, weight, condition, sex, etc.). The dosage requirements needed to achieve the “effective amount” vary with the particular formulations employed, the route of administration, and clinical objectives. Based on the results obtained in standard pharmacological test procedures, projected daily dosages of active compound can be determined as is understood in the art.

Any suitable form of administration can be employed in connection with the diagnostic and therapeutic formulations of the invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The diagnostic and therapeutic formulations of this invention can be administered alone, but may be administered with a pharmaceutical carrier selected upon the basis of the chosen route of administration and standard pharmaceutical practice.

The diagnostic and therapeutic formulations of this invention and medicaments of this invention may further comprise one or more pharmaceutically acceptable carrier, excipient, buffer, emulsifier, surfactant, electrolyte or diluent. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985).

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of excludes any element, step, or ingredient not specified in the claim element. As used herein, “consisting essentially of does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A compound being of the formula (FX1): 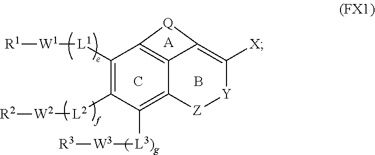
wherein:
Y is —CU^{a}U^{b}—, —NU^{a}—, —O—, —S—, or —C(O)—;
Z is —CU^{c}U^{d}—, —NU^{c}—, —O—, S—, or —C(O)—;
wherein each U^{a} is independently -(L⁴)ₕ-W⁴—R⁴;
wherein each U^{b} is independently -(L⁵)ᵢ-W⁵—R⁵;
wherein each U^{c} is independently -(L⁶)ⱼ-W⁶—R⁶;
wherein each U^{d} is independently -(L⁷)ₖ-W⁷—R⁷;
X is hydrogen, F, Cl, Br, I, or At;
Q is —C(R⁸R⁹)N═N—, —C(R⁸)═NN(R⁹)—, or —N═N—,
each of L¹-L⁷, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
each of W¹-W⁷ is independently a single bond, —(CH₂)ₙ—, —(HCCH)ₙ—, —O—, —S—, —SO—, —SO₂—, —SO₃—, —OSO₂—, —NR¹⁴—, —CO—, —COO—, —OCO—, —OCOO—, —CONR¹⁵—, —NR¹⁶CO—, —OCONR¹⁷—, —NR¹⁸COO—, —NR¹⁸CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ—, —OCO(CH₂)ₙ—, —OCOO(CH₂)ₙ—, —CONR²⁴(CH₂)ₙ—, —CONR²⁵(CH₂)ₙ—, —NR²⁸CO(CH₂)ₙ—, —OCONR²⁷(CH₂)ₙ—, —NR²⁸COO(CH₂)ₙ—, —NR²⁸CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—;
each of R¹-R⁹ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₂₀ alkoxy, C₁-C₂₀ alkoxycarbonyl, C₁-C₂₀ alkoxyalkyl, C₁-C₁₀ polyhydroxyalkyl, C₁-C₁₀ polyalkoxyalkyl, halo, halomethyl, dihalomethyl, trihalomethyl, —CO₂R⁴⁰, —SOR⁴¹, —OSR⁴², —SO₂OR⁴³, —CH₂(CH₂OCH₂)_{b}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —SO₃R⁶², —CH(R⁶³)CO₂H, —CH(R⁶⁴)NH₂, FL or Bm; or wherein R¹, R², W¹, W², and L¹ and L², if present, together with the atoms to which they are attached combine to form one or more carbocyclic or heterocyclic 5, 6, or 7 membered rings; or wherein R², R³, W², W³, and L² and L³, if present, together with the atoms to which they are attached combine to form one or more carbocyclic or heterocyclic 5, 6, or 7 membered rings; or wherein U³ and U^{c} together with the atoms to which they are attached combine to form one or more carbocyclic or heterocyclic 5, 6, or 7 membered rings; or wherein R³, W³, and L³, if present, and U^{c} together with the atoms to which they are attached combine to form one or more carbocyclic or heterocyclic 5, 6, or 7 membered rings;
each of a and b is independently an integer selected from the range of 1 to 100;
each of n is independently an integer selected from the range of 1 to 10;
each of e, f, g, h, i, j, and k is independently 0 or 1;
each of R¹⁴-R³⁶ is independently hydrogen, C₁-C₁₀ alkyl, C₁-C₁₀ alkoxy, C₅-C₁₀ aryl, C₁-C₁₀ alkoxyalkyl, C₁-C₁₀ polyhydroxyalkyl, —(CH₂)ₙCO₂R⁶⁵, or —COR⁶⁶;
each of R⁴⁰-R⁶² and R⁶⁵-R⁶⁶ is independently hydrogen, C₁-C₁₀ alkyl, C₁-C₁₀ alkoxy, C₁-C₁₀ alkoxyalkyl, or C₁-C₁₀ polyhydroxyalkyl;
each of R⁶³ and R⁶⁴ is independently a side chain residue of a natural α-amino acid;
each of FL is independently a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine; and
each Bm is independently a group corresponding to an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steriod, a hormone, an aptamer, a receptor, a metal chelating agent, a mono- or polynucleotide comprising 1 to 50 nucleic acid units, or a polypeptide comprising 2 to 30 amino acid units.

2. The compound of claim 1, being of the formula (FX2):

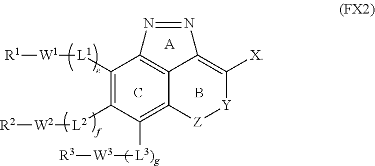

3. The compound of claim 1, being of the formula (FX3) or (FX4):

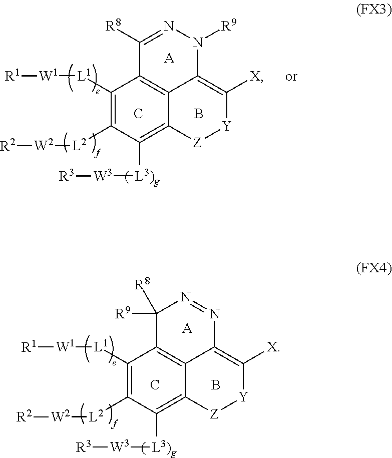

4. The compound of claim 1, being of the formula (FX5), (FX6), (FX7) or (FX8):

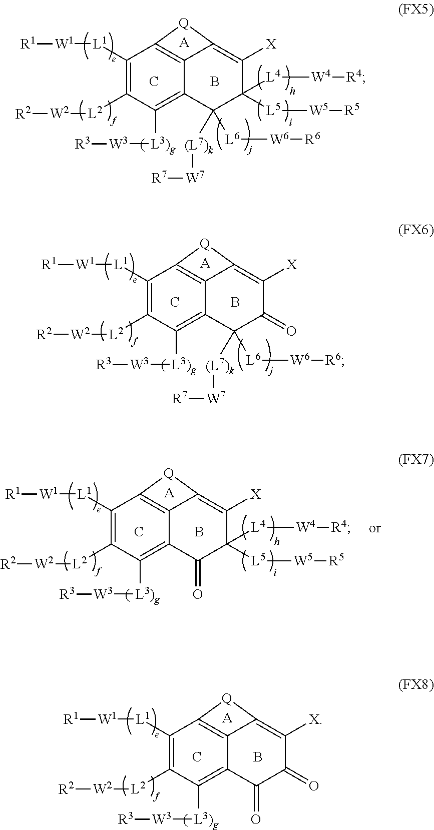

5. The compound of claim 1, being of the formula (FX9),(FX10), (FX11), (FX12), (FX13) (FX14), (FX16), (FX16), (FX17), (FX18), (FX19) or (FX20):

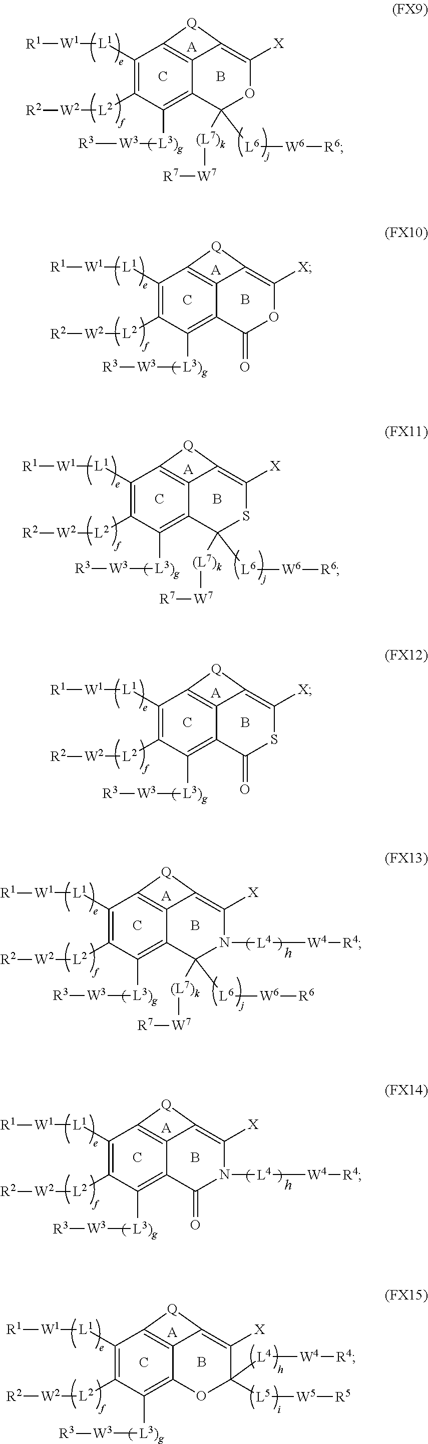

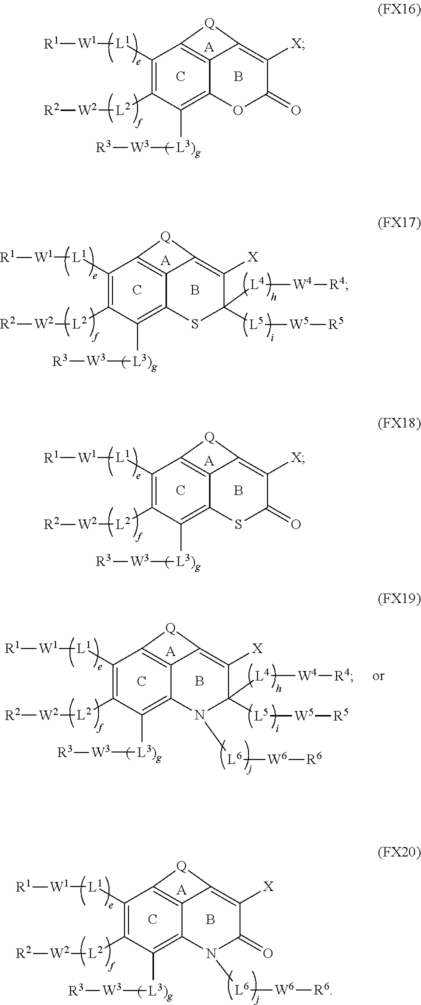

6. The compound of claim 1, wherein R¹, R², W¹, W², and L¹ and L², if present, together with the atoms to which they are attached combine to form one or more carbocyclic or heterocyclic 5, 6, or 7 membered rings; or wherein R², R³, W², W³, and L² and L³, if present, together with the atoms to which they are attached combine to form one or more carbocyclic or heterocyclic 5, 6, or 7 membered rings; or wherein U^{a} and U^{c} together with the atoms to which they are attached combine to form one or more carbocyclic or heterocyclic 5, 6, or 7 membered rings; or wherein R³, W³, and L³, if present, and U^{c} together with the atoms to which they are attached combine to form one or more carbocyclic or heterocyclic 5, 6, or 7 membered rings; the compound being of the formula (FX21), (FX22), (FX23), (FX24), (FX25), (FX26), (FX27) or (FX28): 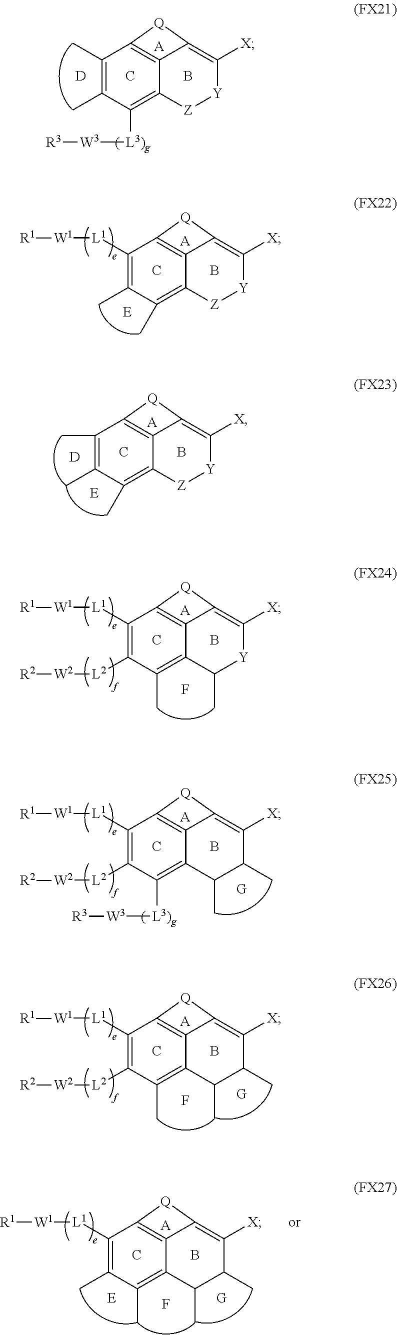 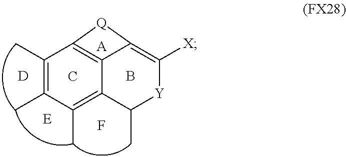
wherein each of rings D, E, F and G is independently said one or more carbocyclic or heterocyclic 5, 6, or 7 membered rings.

7. The compound of claim 6, wherein each of rings D and E is independently a carbocyclic or heterocyclic C₅-C₂₀ aryl group fused to ring C.

8. The compound of claim 7, wherein each of rings D and E is independently a group corresponding to benzene, naphthalene, naphthoquinone, diphenylmethane, fluorene, anthracene, anthraquinone, phenanthrene, tetracene, naphthacenedione, pyridine, quinoline, isoquinoline, indole, isoindole, pyrrole, imidazole, oxazole, thiazole, pyrazole, pyrazine, pyrimidine, purine, benzimidazole, furan, benzofuran, dibenzofuran, carbazole, acridine, acridone, phenanthridine, thiophene, benzothiophene, dibenzothiophene, xanthene, xanthone, flavone, coumarin, azulene, aza-azulene, or anthracycline.

9. The compound of claim 6, wherein each of rings F and G is independently a carbocyclic or heterocyclic C₅-C₁₀ cycloalkyl or C₅-C₁₀ cycloalkenyl fused to ring B.

10. The compound of claim 9, wherein each of rings F and G is independently cyclopentane, cyclohexane, cycloheptane or piperidine.

11. The compound of claim 1, wherein X is a halogen.

12. The compound of claim 1, wherein X is F, Cl, Br.

13. The compound of claim 1, wherein X is Br.

14. The compound of claim 1, wherein at least one of R¹-R⁹ is Bm.

15. The compound of claim 1, wherein at least one of R⁴-R⁷ is Bm.

16. The compound of claim 1, wherein at least one of R¹-R⁹ is FL.

17. The compound of claim 1, wherein at least one of R¹-R³ is an electron donating group, and wherein at least one of R¹-R³ is an electron withdrawing group.

18. The compound of claim 1, wherein at least one of R¹-R³ is C₁-C₁₀ alkyl, —OR⁴⁶, —SR⁴⁷, —NR⁴⁸R⁴⁹, or —NR⁵⁰COR⁵¹.

19. The compound of claim 1, wherein at least one of R¹-R³ is —CN, halo, —CO₂R⁴⁰, —COR⁵⁴, —NO₂, —SO₂R⁵⁵, or —SO₂NR⁵⁸R⁵⁹.

20. The compound of claim 1, wherein at least one of R¹-R³ is C₁-C₁₀ alkyl, —OR⁴⁶, —SR⁴⁷, —NR⁴⁸R⁴⁹, or —NR⁵⁹COR⁵¹, and at least one of R¹-R³ is —CN, halo, —CO₂R⁴⁹, —COR⁵⁴, —NO₂, —SO₂R⁵⁵, or —SO₂NR⁵⁹R⁵⁹.

21. The compound of claim 1 being of the formula (FX29), (FX30) (FX31), (FX33) or (FX40):

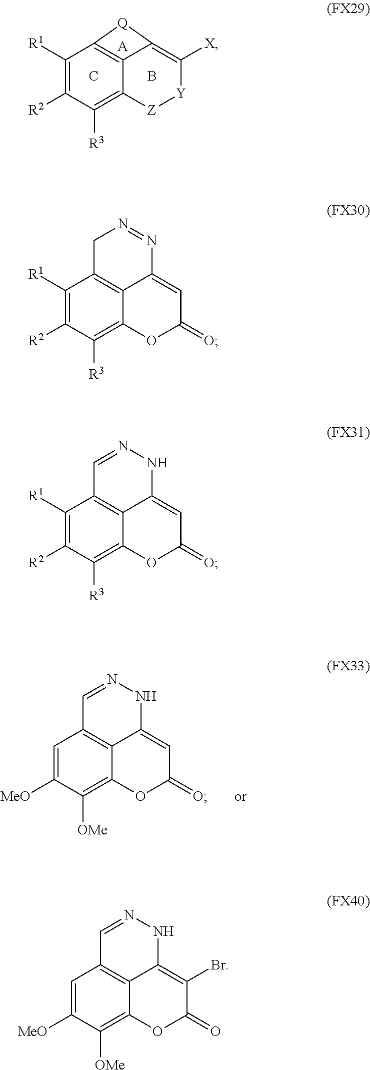

22. A method for a phototherapy procedure, the method comprising:
administering to a subject in need of treatment a therapeutically effective amount of a compound being of the formula (FX1), or a pharmaceutical formulation thereof; and
exposing the administered compound to electromagnetic radiation, wherein: 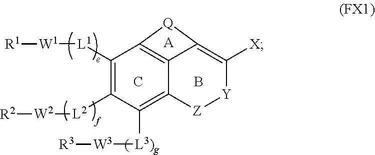
wherein:
Y is —CU^{a}U^{b}—, —NU^{a}—, —O—, —S—, or —C(O)—;
Z is —CU^{c}U^{d}—, —NU^{c}—, —O—, S—, or —C(O)—;
wherein each U^{a} is independently -(L⁴)ₕ-W⁴—R⁴;
wherein each U^{b} is independently -(L⁵)ᵢ-W⁵—R⁵;
wherein each U^{c} is independently -(L⁶)ⱼ-W⁶—R⁶;
wherein each U^{d} is independently -(L⁷)ₖ-W⁷—R⁷;
X is hydrogen, F, Cl, Br, I, or At;
Q is —C(R⁸R⁹)N═N—, —C(R⁸)═NN(R⁹)—, or —N═N—,
each of L¹-L⁷, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
each of W¹-W⁷ is independently a single bond, —(CH₂)ₙ—, —(HCCH)ₙ—, —O—, —S—, —SO—, —SO₂—, —SO₃—, —OSO₂—, —NR¹⁴, —CO—, —COO—, —OCO—, —OCOO—, —CONR¹⁵—, —NR¹⁶CO—, —OCONR¹⁷—, —NR¹⁸COO—, —NR¹⁹CONR²⁰—, —NR²¹CSNR²²—, —O(CH₂)ₙ—, —S(CH₂)ₙ—, —NR²³(CH₂)ₙ—, —CO(CH₂)ₙ—, —COO(CH₂)ₙ—, —OCO(CH₂)ₙ—, —OCOO(CH₂)ₙ—, —CONR²⁴(CH₂)ₙ—, —CONR²⁵(CH₂)ₙ—, —NR²⁸CO(CH₂)ₙ—, —OCONR²⁷(CH₂)ₙ—, —NR²⁸COO(CH₂)ₙ—, —NR²⁹CONR³⁰(CH₂)ₙ—, —NR³¹CSNR³²(CH₂)ₙ—, —O(CH₂)ₙNR³³CO(CH₂)ₙ—, —CO(CH₂)ₙ(CH₂OCH₂)ₙ(CH₂)ₙNR³⁴(CH₂)ₙNR³⁵CO—, -or —CO(CH₂)ₙNR³⁶CO—;
each of R¹-R⁹ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, alkoxy, C₁-C₂₀ alkoxycarbonyl, C₁-C₂₀ alkoxyalkyl, C₁-C₁₀ polyhydroxyalkyl, C₁-C₁₀ polyaikoxyalkyl, halo, halomethyl, dihalomethyl, trihalomethyl, —CO₂R⁴⁰, —SOR⁴¹, —OSR⁴² , —SO₂OR⁴³, —CH₂(CH₂OCH₂)_{b}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹,—SO₃R⁶², —CH(R⁶³)CO₂H, —CH(R⁶⁴)NH₂, FL or Bm; or wherein R¹, R², W¹, W², and L¹ and L², if present, together with the atoms to which they are attached combine to form one or more carbocyclic or heterocyclic 5, 6, or 7 membered rings; or wherein R², R³, W², W³, and L² and L³, if present, together with the atoms to which they are attached combine to form one or more carbocyclic or heterocyclic 5, 6, or 7 membered rings; or wherein U^{a} and U^{c} together with the atoms to which they are attached combine to form one or more carbocyclic or heterocyclic 5, 6, or 7 membered rings; or wherein R³, W³, and L³, if present, and U^{c} together with the atoms to which they are attached combine to form one or more carbocyclic or heterocyclic 5, 6, or 7 membered rings;
each of a and b is independently an integer selected from the range of 1 to 100;
each of n is independently an integer selected from the range of 1 to 10;
each of e, f, g, h, i, i, and k is independently 0 or 1;
each of R¹⁴-R³⁶ is independently hydrogen, C₁-C₁₀ alkyl, C₁-C₁₀ alkoxy, C₅-C₁₀ aryl, C₁-C₁₀ alkoxyalkyl, C₁-C₁₀ polyhydroxyalkyl, —(CH₂)ₙCO₂R⁶⁵, or —COR⁶⁶;
each of R⁴⁰-R⁶² and R⁶⁵-R⁶⁶ is independently hydrogen, C₁-C₁₀ alkyl, C₁-C₁₀ alkoxy, C₁-C₁₀ alkoxyalkyl, or C₁-C₁₀ polyhydroxyalkyl;
each of R⁶³ and R⁶⁴ is independently a side chain residue of a natural α-amino acid;
each of FL is independently a group corresponding to a pyrazine, a thiazole, a phenylxanthene, a phenothiazine, a phenoselenazine, a cyanine, an indocyanine, a sguaraine, a dipyrrolo pyrimidone, an anthraquinone, a tetracene, a quinoline, an acridine, an acridone, a phenanthridine, an azo dye, a rhodamine, aphenoxazine, an azulene, an aza-azulene, a triphenyl methane dye, an indole, a benzoindole, an indocarbocyanine, a Nile Red dye, or a benzoindocarbocyanine; and
each Bm is independently a group corresponding to an amino acid, a peptide, a protein, a nucleoside, a nucleotide, an enzyme, a carbohydrate, a plycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steriod, a hormone, an aptamer, a receptor, a metal chelating agent, a mono- or polynucleotide comprising 1 to 50 nucleic acid units, or a polypeptide comprising 2 to 30 amino acid units.

23. The method of claim 22, wherein said procedure is a Type 1 phototherapy procedure.

24. The method of claim 22, wherein the method comprises exposing the administered compound to electromagnetic radiation having wavelengths selected over a range of 350 nanometers to 1300 nanometers.

25. The method of claim 22, wherein exposing the administered compound to electromagnetic radiation generates a therapeutically effective amount of photoactivated compound.

26. The method of claim 22, wherein exposing the administered compound to electromagnetic radiation cleaves a C—N, N═N or C—X bond of the compound.

27. The method of claim 22, wherein exposing the administered compound to electromagnetic radiation generates a therapeutically effective amount of reactive species causing localized cell death or injury.

28. The method of claim 22, wherein the method comprises contacting a target tissue of the subject with the administered compound.

29. The method of claim 28, wherein the target tissue is colon, prostate, gastric, esophageal, uterine, endometrial, pancreatic, breast, cervical, brain, skin, gallbladder, lung, throat, kidney, testicular, prostate, gastric, or ovary tissue.

30. The method of claim 28, wherein the target tissue is cancerous tissue.

31. The method of claim 28, wherein the target tissue is a tumor.

32. The method of claim 22, for use in treatment of cancer or a cancer-associated disorder.

33. The method of claim 32, wherein the cancer or cancer-associated disorder is colon cancer, prostate cancer, gastric cancer, esophageal cancer, uterine cancer, endometrial cancer, pancreatic cancer, breast cancer, cervical cancer, brain cancer, skin cancer, gallbladder cancer, lung cancer, or ovarian cancer.

34. The method of claim 22, for use in treatment of inflammation or an inflammation -associated disorder.

35. A pharmaceutical composition comprising:
the compound of claim 1; and
one or more pharmaceutically acceptable excipients.

36. A pharmaceutical composition comprising:
the compound of claim 1, or a pharmaceutical composition thereof; and
one or more additional therapeutic agents and/or diagnostic agents.
